(19) United States
(12) Patent Application Publication
(10) Pub. No.: US 2025/0302728 A1
(43) Pub. Date: Oct. 2, 2025

(54) FRAGRANCE COMPOSITIONS AND PRODUCTS CONVEYING A POSITIVE MOOD

(71) Applicant: SYMRISE AG, Holzminden (DE)

(72) Inventors: Patrice BELLON, Issy Les Moulineaux (FR); Arnaud AUBERT, Tours (FR); Francis VIAL, Tours (FR); Celine CARRASCO-DOUROUX, Le Vesinet (FR); Gaëlle LE GOFF, Wien (AT); Lisa LE MAUFF, Paris (FR); Patricia ARNOSTTI NEPOMUCENO, Paris (FR); Edison DIAZ, Goslar (DE); Jonathan JACOBS, Singapore (SG); Katharine A. PRIGGE, West Caldwell, NJ (US); Keith MCDERMOTT, Bound Brook, NJ (US)

(73) Assignee: SYMRISE AG, Holzminden (DE)

(21) Appl. No.: 18/864,067

(22) PCT Filed: May 17, 2023
(86) PCT No.: PCT/EP2023/063237

§ 371 Date: Nov. 8, 2024

(30) Foreign Application Priority Data
May 17, 2022 (WO) PCT/US2022/029607

(51) Int. Cl. A61K 8/92; A61K 8/34; A61K 8/35; A61K 8/37; A61K 8/49; A61Q 13/00

(52) U.S. Cl. CPC A61K 8/922; A61K 8/34; A61K 8/35; A61K 8/37; A61K 8/4973; A61Q 13/00

(57) ABSTRACT

The present invention primarily relates to the use of a fragrance composition for conveying a positive mood in a subject, preferably a human, as well as to the use of a product containing the fragrance composition. Furthermore, the present invention relates to a method for imparting a positive mood and fragrance compositions for conveying a positive mood in a subject, preferably a human.

The present invention primarily relates to the use of a fragrance composition (either per se or contained in a perfumed product) for conveying a positive mood in a subject, preferably a human. Furthermore, the present invention relates to a method for imparting a positive mood in a subject, preferably a human.

Also described herein are novel fragrance compositions for conveying a positive mood. Further aspects of the present invention will arise from the description below, in particular from the examples, as well as from the attached patent claims.

Many products available on the market are advertised having an “energizing” or “calming” benefit to the end user. Olfactory information is believed to be carried directly from the olfactory bulb into the limbic system, or the emotional heart of the brain. Therefore, scent plays a powerful role in evoking and reinforcing emotions and fragrance is the ideal vehicle to deliver positive mood enhancement to an individual. Aside from descriptions of aromatherapy, however, there are few cited reports that reveal how to design fragrance that are optimized both for hedonic acceptance and shown to deliver targeted emotional benefit.

In the context of the present invention, reference is made to prior art document EP 1829559 A1 relating to methods for improving sleep behaviours. Furthermore, reference is made to US 20080096790 A1 relating to fragrance compositions with beneficial effects, US 20100174586 A1 relating to methods for measuring emotive response and selection preference to visual stimuli, US 20040033279 relating to a process for effecting the relaxation of muscles of a human by means of a fragrance, EP 2158895 B1 relating to a method for energizing human beings, U.S. Pat. No. 6,830,755 B2 relating to a method for relaxing human beings using personal care compositions, U.S. Pat. No. 8,435,572 B2 relating to compositions and methods for promoting a relaxation state, EP 1293554 A1 relating to stress relieving perfumes and stress relieving perfume compositions containing the same, and WO 2002049600 A1 relating to perfume compositions which aim to promote relaxed and related mood states in subjects. WO2018206297 A1 discloses a fragrance for enhancing the mood in a subject, in particular enhancing the mood of a baby and of the mother.

While previous attempts measure the level of excitation or energizing effect (e.g. EP 2158895 B1) of a single substance, few fragrances have been evaluated for their direct effect to elicit a specific mood benefit.

Untrained individuals often cannot tell (due to lack of ability or vocabulary) or will not tell how they really feel about a product, especially when they are receiving compensation for testing or evaluation. Therefore relying heavily on interviews, questionnaires, or self-assessment psychometric tools to measure true human emotion is problematic. In the study underling the present invention, a combined analysis using vocal analysis (prosody), semantic analysis (verbatim), psychometric scale on the Genova Emotional Wheel (GEW), an implicit response test (IRT) and an emotional characterization (EmoChar™) was used to determine the mood-altering effects of fragrance. As humans have evolved as highly social creatures, individuals spontaneously and subconsciously communicate through verbal. Evaluation of these verbal responses to a fragrance stimuli, allows for the subconscious and unaltered evaluation of human emotion in response to scent.

The study underlying the present invention has investigated the emotional influence of more than 250 fragrance raw materials. These test results were then used to create a toolbox for using the information for fragrance raw materials to create fragrance accords, which can be used in fragrance compositions for conveying a positive mood.

The state of the art discloses only limited possibilities for a scientific approach for creating a fragrance having desired properties in terms of their influence on the mood of a subject.

It was the primary object of the present invention to provide a targeted assessment of fragrance raw materials and their evaluation on the mood of a subject to design target-oriented fragrance compositions for imparting a certain mood in a subject.

Further objects underlying the present invention follow from the description below and the attached patent claims.

The primary object of the present invention is solved by providing the use of a fragrance composition for conveying a positive mood, preferably an attractive, protective, joyful, fresh, energizing, relaxing, sensual, self-confident and/or well-being mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 3-(4-tert-butylphenyl)propanal, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-Trimethyl-benzenepropanol, methyl-2-aminobenzoate, (E)-4-(2,6,6-trimethylcyclohex-2-en-1-yl)but-3-en-2-one, 3-(4-ethylphenyl)-2,2-dimethylpropanal, 3-(1,3-benzodioxol-5-yl)-2-methyl-propanal, (2E)-3,7-dimethylocta-2,6-dien-1-ol, methyl-2-[RAC-(1S,2S)-3-oxo-2-pentyl-cyclopentyl]acetate, 1-phenylethyl acetate, benzyl acetate, 1,3-benzodioxole-5-carbaldehyde, [(Z)-hex-3-enyl]-2-hydroxybenzoate, (methyl-2-[RAC-(1S,2S)-3-oxo-2-pentyl-cyclopentyl]acetate, (1,5-dimethyl-1-vinyl-hex-4-enyl)acetate, 2-phenylethanol, 4-Isopentylcyclohexanol, benzyl 2-hydroxybenzoate, 4-cyclohexylbutan-2-ol, (E)-1-(2,6,6-trimethylcyclohexan-1,3-dien-1-yl)but-2-en-1-one, (2E)-3,7-dimethylocta-2,6-dienyl]acetate, (E)-4-(2,6,6-trimethylcyclohexen-1-yl)but-3-en-2-one, (+)-3-(4-Isopropylcyclohexen-1-yl)propanal, 2,6-dimethyloct-7-en-2-ol 7-methyl-1,5-benzodioxepin-3-one, 1-methyl-4-(4-methylpent-3-enyl)cyclohex-3-ene-1-carbaldehyde, (E)-1-(2,6,6-trimethylcyclohex-2-en-1-yl)but-2-en-1-one, 2-isobutyl-4-methyl-tetrahydropyran-4-ol, [(2E)-3,7-dimethylocta-2,6-dienyl]acetate, indole, (1E)-1-(2,6,6-trimethylcyclohex-2-en-1-yl)hepta-1,6-dien-3-one, methyl 2-[(7-hydroxy-3,7-dimehyloctylidene)amino]benzoate, rac-(e)-1-[rac-(1r,2s)-2,6,6-trimethylcyclohex-3-en-1-yl]but-2-en-1-one, 2-methyl-1-phenyl-propan-2-ol, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 2,3-dihydro-1,1-dimethyl-1h-indene-ar-propanal, (E)-1-(2,4,4-trimethylcyclohexen-1-yl)but-2-en-1-one, (2E)-2-methyl-6-methylene-octa-2,7-dien-1-ol, 5-hexyl-5-methyl-tetrahydrofuran-2-one, 3,7-dimethylocta-1,6-dien-3-ol, 4-(propan-2-yl)cyclohexyl]methanol, 1-naphthalen-2-ylethanone, 2-ethoxynaphtalene, 2-methoxynaphtalene, 3-methyl-2-benzofuran-1-yl)ethanone, 2-phenoxyethyl 2-methylpropanoate, 3-methyl-5-phenylpentan-1-ol, 2-methyl-4-phenyl-butan-2-ol, (2,2,2-trichloro-1-phenyl-ethyl) acetate, 2-methyl-5-phenylpentanol, rac-(2r,4s)-4-methyl-2-(2-methylprop-1-enyl)tetrahydropyran, 2,2,5-trimethyl-5-pentyl-cyclopentanone, 3-(4-methoxyphenyl)-2-methyl-propanal, (6E)-3,7,11-trimethyldodeca-1,6,10-trien-3-ol, (6Z)-3,7,11-trimethyldodeca-1,6,10-trien-3-ol and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-tetramethyldodecahydronaphtho[2,1-b]furan, 1,1,2,3,3,-pentamethyl-2,5,6,7-tetrahydroinden-4-one, 8-methoxy-2,6,6,8-tetramethyl-tricyclo[5.3.1.01,5]undecane, (E)-3-methyl-4-(2,6,6-trimethylcyclohex-2-en-1-yl)but-3-en-2-one, (2E)-2-Ethyl-4-(2,2,3)-trimethylcyclopent-3-en-1-yl)but-2-en-1-ol, 1-[(2E,5E,9E)-2,6,10-trimethylcyclododeca-2,5,9-trien-1-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, 1-[2,3,8,8-tetramethyl-1,3,4,5,6,7-hexanhydronaphthalen-2-yl]ethanone, (Ethoxymethoxy)cyclododecane, dioxol-4H-4A,9-methanoazuleno(5,6-D)-octahydro-2-propyl-2,5,8,8,9A-pentamethyl-1,3, (E/Z)-4-Ethyloct-2/3-enenitrile, 3,4-dimethylbenzonitrile, (2E)-3-phenylprop-2-en-1-ol, 2,3,6-trimethylcyclohexanol, oxacycloheptadec-10-en-2-one, (4aR,5R,7aS,9R)-octahydro-2,2,5,8,8,9a-hexamethyl-4H-4a,9-methanoazuleno(5,6-d)-1,3-dioxole, 2-ethoxy-2,6,6-trimethyl-9-methylene-bicyclo[3.3.1]nonane, (E)-2-methyl-3-phenyl-prop-2-en-1-ol, rac-(E,2R,3R)-3-methyl-5-(2,2,3-trimethylcyclopent-3-en-1-yl)pent-4-en-2-ol, 4-(1,7,7-trimethyl-2-bucyclo[3.1.1]heptanyl)ccyclohexan-1-ol, 6-butan-2-ylquinoline, 4-(1-ethoxyvinyl)-3,3,5,5-tetramethyl-cyclohexanone, (E)-3,4,5,6,6-pentamethylhept-3-en-2-one, 2-(4-methylcyclohex-3-en-1-yl)propan-2-ol, 1-(2,6,6,8-tetramethyl-9-tricyclo[5.3.1.01,5]undec-8-enyl)ethanone, rac-(3′R,6′R,9′R)-2′,2′,8′,8′-tetramethylspiro[1,3-dioxolane-2,10′-octahydro-1h-2,4a-methanonapthalene], 1-(1,1,2,4,4,7-hexamethyltetralin-6-yl)ethanone, Oxacycloheptadec-10-en-2-one, DL Ambre 83 SMP N, Methyl-2,4-dihydroxy-3,6-dimethyl-benzoate, (4-tert-butylcyclohexyl) acetate, and mixtures thereof,
and
at least one substance of group (C) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (C) consists of compounds having an earthy and/or sweet odor impression selected from the group consisting of chromen-2-one, 2-ethyl-3-hydroxy-pyran-4-one, (5Z)-3-methylcyclopentadec-5-en-1-one, 3-ethoxy-4-hydroxy-benzaldehyde, 1,4-dioxacycloheptadecane-5,17-dione, 4,6,6,7,8,8-hexamethyl-1,3,4,7-tetrahydrocyclopenta[G]isochromene, oxacyclohexadecen-2-one (mixture of isomers), Pentadecan-15-olide, [2-[1-(3,3-dimethylcyclohexyl)ethoxy]-2-methyl-propyl]propanoate, 3,6-Dimethyl-3H-benzofuran-2-one, (5Z)-3-methylcyclopentadec-5-en-1-one, 4,6,6,7,8,8-hexamethyl-1,3,4,7-tetrahydrocyclopenta[G]isochromene, 2-methyl-5-propyl-cyclohex-2-en-1-one, 1,3-benzodioxole-5-carbaldehyde, 1-pyrazin-2-ylethanone, 5-pentyltetrahydrofuran-2-one, 1-methoxy-4-[(e)-prop-1-enyl]benzene, 4-methoxybenzaldehyde, 4-hydroxy-2,5-dimethylfuran-3-one, 3-hydroxy-2-methyl-4h-pyran-4-one, 2-(4-methylthiazol-5-yl)ethanol, 4-hydroxy-3-methoxy-benzaldehyde, (4-formyl-2-methoxy-phenyl)-2-methylpropanoate, and mixtures thereof,
and
at least one essential oil and/or essence and/or absolue selected from the group consisting of bergamot oil, ginger root extract, vetiver oil, lemon oil, artemisia oil, cinnamon bark oil, elemi oil, ginger oil, lemongrass oil, longoza oil, mandarin oil, pepper black oil, vanilla absolue, red berry extract, bay oil, cedarwood oil, hemp oil, pink pepper leaf oil, amyris oil, cardamom oil, carrot seed oil, cypress oil, davana oil, geranium oil, grapefruit oil, lime oil, olibanum oil, palmarosa oil, sage oil, sandalwood oil, thyme oil, artichoke essence, asparagus essence, cauliflower essence, hemp essence, leek essence, clove essence, passionfruit essence, amber base, red apple essence, benzaldehyde, Styrax tonkinensis essence, camphor oil, eucalyptus oil, jasmin oil, jasmin absolue, orange oil, rosemary oil, galbanum essence, orange flower absolue, patchouli essence, rose absolue, tonka bean absolue, tuberose absolue and rose bourboniana absolute.

It is preferred in terms of the present invention if the artichoke essence, asparagus essence, cauliflower essence, leek essence and clove essence are obtained according to the SymTrap® extraction method, e.g. as described in EP2385098B1.

In a preferred embodiment, the at least one essential oil is contained in an amount of from 0.01 wt-% to 30 wt.-%, preferably of from 0.1 wt.-% to 15 wt.-% and especially preferably in an amount of from 1 wt.-% to 5 wt.-%, in each case based on the total amount of the fragrance composition.

It was surprisingly found that a fragrance composition comprising at least one substance from each group is able to convey a positive mood. The preferable positive mood(s) is/are selected from the group consisting of an attractive, a protective, a fresh, an energizing, a joyful, a relaxing, a sensual, a self-confident and a well-being mood.

An “attractive mood” in terms of the present invention describes a stimulus that makes the subject feel more attractive to other people. The subject has the perception that another person will find it physically and intellectually attractive.

A “protective mood” in terms of the present invention describes a stimulus that triggers the sensation of being protected from hazardous components from environment.

A “fresh mood” in terms of the present invention describes a stimulus that triggers the sensation of being refreshed and invigorated and feeling youthful and lively.

An “energizing mood” in terms of the present invention describes the degree to which the sensory stimulus is both arousing and pleasurable.

A “joyful mood” in terms of the present invention describes a stimulus that triggers caring for others, gratitude, delightfulness, thankfulness and is more long lasting than happiness.

A “relaxing mood” in terms of the present invention describes the degree to which the sensory stimulus is lacking in arousal, but is pleasurable. Substances, which were assessed to convey a relaxing mood, have a beneficial effect on the sleeping quality of a subject, too.

A“sensual mood” in terms of the present invention describes a stimulus that arouses sexual instinct by increasing pleasure, desire or performance in a subject.

A “self-confident mood” in terms of the present invention describes a stimulus that triggers the confirmation skills and abilities. This means, the subject accept himself and has more trust in himself, thus provoking a sense of control.

A “well-being mood” in terms of the present invention describes a general stimulus conveying happiness, comfort, mental and physical health.

The moods as described herein can further be regarded as arousing or calming moods.

“Arousing moods” are moods, which convey an active stimulus, preferably activating the sympathetic nervous system of the subject. In particular, attractive, protective, fresh, energizing, joyful and a self-confident mood are arousing moods.

“Calming moods” are moods, which convey a passive stimulus and thus have an impact on the parasympathetic nervous system. In particular, a relaxing, a sensual and a well-being mood are calming moods.

A preferable embodiment of the present invention relates to the use of a fragrance composition for conveying a positive mood, preferably a protective mood, in a subject, preferably a human, wherein the fragrance composition comprises or consists of:

Optionally at least one substance of group (A) in a total amount of between 0.01 and 70 wt.-%, preferably of between 0.2 and 50 wt.-%, especially preferably of between 0.5 and 35 wt.-%,
wherein group (A) consists of compounds with a bloomy odor impression selected from 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-trimethyl-benzenepropanol, methyl-2-aminobenzoate, (E)-4-(2,6,6-trimethylcyclohex-2-en-1-yl)but-3-en-2-one, methyl 2-[(7-hydroxy-3,7-dimethyloctylidene)amino]benzoate, 2,6-dimethylheptan-2-ol, 5-hexyl-5-methyl-tetrahydrofuran-2-one, 3,7-dimethylocta-1,6-dien-3-ol, 4-(propan-2-yl)cyclohexyl]methanol, 3-(4-methoxyphenyl)-2-methyl-propanal and mixtures thereof, preferably selected from 2,6-dimethylheptan-2-ol,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 1,1,2,3,3,-pentamethyl-2,5,6,7-tetrahydroinden-4-one, 4,6,6,7,8,8-hexamethyl-1,3,4,7-tetrahydrocyclopenta[g]isochromene, oxacyclohexadecen-2-one (mixture of isomers), pentadecan-15-olide, [2-[1-(3,3-dimethylcyclohexyl)ethoxy]-2-methyl-propyl]propanoate, 1-[(2E,5E,9E)-2,6,10-trimethylcyclododeca-2,5,9-trien-1-yl]ethanone, 2,2,6,8-tetramethyltricyclo[5.3.1.03,8]undecan-3-ol, 1-(2,6,6,8-tetramethyl-9-tricyclo[5.3.1.01,5]undec-8-enyl)ethanone, and mixtures thereof,
and
at least one substance of group (C) in a total amount of between 0.01 and 99 wt.-%, preferably of between 0.01 and 35 wt.-%, preferably of between 0.2 and 20 wt.-%, especially preferably of between 0.5 and 10 wt.-%, wherein group (C) consists of compounds having an earthy and/or sweet odor impression selected from the group consisting of 3-ethoxy-4-hydroxy-benzaldehyde, (4-formyl-2-methoxy-phenyl) 2-methylpropanoate and mixtures thereof, and mixtures thereof;
and
at least one essential oil and/or essence and/or absolue selected from the group consisting of lavender oil, eucalyptus oil, geranium oil, rose absolue, sage oil and tonka bean absolue.

One preferred embodiment of the present invention relates to the use of a fragrance composition for conveying a positive mood, preferably a sensual mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 20 wt.-%, moreover preferably of between 1 and 20 wt.-%,
wherein group (A) consists of compounds with a bloomy odor impression selected from the group consisting of 2,6-dimethyloct-7-en-2-ol, beta, beta-3-Trimethyl-benzenepropanol and mixtures thereof, and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, 1,4-dioxacycloheptadecane-5,17-dione, 8-methoxy-2,6,6,8-tetramethyl-tricyclo[5.3.1.01,5]undecane, 1-[2,3,8,8-tetramethyl-1,3,4,5,6,7-hexanhydronaphthalen-2-yl]ethanone, 2,2,6,8-tetramethyltricyclo[5.3.1.03,8]undecan-3-ol, 2-ETHOXY-2,6,6-trimethyl-9-methylene-bicyclo[3.3.1]nonane, 4-(1-ethoxyvinyl)-3,3,5,5-tetramethyl-cyclohexanone, 1-(2,6,6,8-tetramethyl-9-tricyclo[5.3.1.01,5]undec-8-enyl)ethanone, 1-(1,1,2,4,4,7-hexamethyltetralin-6-yl)ethanone, and mixtures thereof;
and
at least one substance of group (C) in a total amount in a total amount of between 0.01 and 70 wt.-%, preferably of between 0.2 and 50 wt.-%, especially preferably of between 0.5 and 35 wt.-%, moreover preferably of between 1 and 20 wt.-%, wherein group (C) consists of compounds having an earthy and/or sweet odor impression selected from the group consisting of 3,6-dimethyl-3H-benzofuran-2-one, 4-hydroxy-3-methoxy-benzaldehyde, 1,4-dioxacycloheptadecane-5,17-dione, (4-formyl-2-methoxy-phenyl) 2-methylpropanoate and mixtures thereof,
and
at least one essential oil and/or essence and/or absolue selected from the group consisting of vetiver oil, vanilla absolue, jasmin absolue, rose absolue, patchouli oil and sandalwood oil.

Another preferred embodiment relates to the use according to the invention, wherein the fragrance composition additionally comprises
at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of allyl 3-cyclohexylpropanoate, 5-hexyltetrahydrofuran-2-one, methoxyphenylbutanone, (2E)-3,7-Dimethylocta-2,6-dienal, 5-heptyltetrahydrofuran-2-one, hexyl acetate, isopropyl tetradeconate, 6,6-dimethoxy-2,5,5-trimethylhex-2-ene, ethyl-3-oxobutanoate, ethyl-2-methylpentanoate, 5-hexyldihydro-4-methylfuran-2(3H)-one, 2-tert-butylcyclohexyl acetate, diethyl 2-methylenebutanedioate, 2-bicyclo[2.2.1]hept-5-enyl acetate, ethyl 3-oxobutanoate, linolal, mixture of diethyl (E/Z)-2-methylbut-2-enedioate and diethyl 2-methylenebutanedioate, mixture of (5-isopropenyl-2-methyl-cyclohex-2-en-1-yl) acetate and (5-isopropenyl-2-methyl-cyclohexyl) acetate, ethyl tetrahydrofuran-2-carboxylate, decanal, ethyl rac-(2r,3r)-3-methyl-3-phenyl-oxirane-2-carboxylate, allyl hexanoate, (1,1-dimethyl-2-phenyl-ethyl) acetate, 5-octyltetrahydrofuran-2-one, ethyl butanoate, ethyl 2-methylbutanoate, 3-methylbutyl acetate, ethyl 2-(2-methyl-1,3-dioxolan-2-yl)acetate, 2,4-dimethyl-4,4a,5,9b-tetrahydoindeno[1,2-d][1,3]dioxine, 2-methyl-4-propyl-1,3-oxathiane, 2,4,6-trimethyl-4-phenyl-1,3-dioxane, allyl heptanoate and mixtures thereof.

Yet another preferred embodiment of the use according to the invention relates to a use according to the invention, wherein the fragrance composition additionally comprises
at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of, allyl-2isopentyloxyacetate, allyl (cyclohexyloxy)acetate, 1-(5,5-dimethylcyclohexen-1-yl)pent-4-en-1-one, 2,4-dimethylcyclohex-3-ene-1-carbaldehyde, (Z)-hex-3-en-1-yl methyl carbonate, hex-5-enyl 2-methylbutanoate, (Z)-hex-3-en-1-ol, [(Z)-hex-3-enyl]acetate, 4,11,11-trimethyl-8-methylene-bicyclo[7.2.0]undec-4-ene, methyl-2,4-dihydroxy-3,6-dimethyl-benzoate, allyl (cyclohexyloxy)acetate, 3,3,5-trimethylcyclohexyl acetate, 1-methyl-4-(4-methylpent-3-enyl)cyclohex-3-ene-1-carbaldehyde, undec-10-enal, 5-isopropenyl-2-methyl-cyclohex-2-en-1-one, 2,4,4,7-tetramethyloct-6-en-3-one, 4-allyl-2-methoxy-phenol, 3a,4,5,6,7,7a-hexahydro-4,7-methanoinden-6-yl acetate, 2,6-dimethylhept-5-enal, 2-ethoxy-4-(methoxymethyl)phenol, methyl 2-nonynoate, (4-tert-butylcyclohexyl) acetate, 5-methylheptan-3-one oxime, (E)-4-methyldec-3-en-5-ol and mixtures thereof.

One preferred embodiment of the present invention relates to the use of a fragrance composition for conveying a positive mood, preferably an attractive mood, in a subject, preferably a human, wherein the fragrance composition comprises or consists of at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-trimethyl-benzenepropanol, methyl-2-aminobenzoate, (E)-4-(2,6,6-trimethylcyclohex-2-en-1-yl)but-3-en-2-one, methyl 2-[(7-hydroxy-3,7-dimethyloctylidene)amino]benzoate, 2,6-dimethylheptan-2-ol, 5-hexyl-5-methyl-tetrahydrofuran-2-one, 3,7-dimethylocta-1,6-dien-3-ol, 4-(propan-2-yl)cyclohexyl]methanol, 3-(4-methoxyphenyl)-2-methyl-propanal and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-tetramethyldodecahydronaphtho[2,1-b]furan, 1,1,2,3,3,-pentamethyl-2,5,6,7-tetrahydroinden-4-one, 1,4-dioxacycloheptadecane-5,17-dione, 4,6,6,7,8,8-hexamethyl-1,3,4,7-tetrahydrocyclopenta[g]isochromene, oxacyclohexadecen-2-one (mixture of isomers), pentadecan-15-olide 8-methoxy-2,6,6,8-tetramethyl-tricyclo[5.3.1.01,5]undecane, [2-[1-(3,3-dimethylcyclohexyl)ethoxy]-2-methyl-propyl]propanoate, (E)-3-methyl-4-(2,6,6-trimethylcyclohex-2-en-1-yl)but-3-en-2-one, (5Z)-3-methylcyclopentadec-5-en-1-one, (2E)-2-ethyl-4-(2,2,3)-trimethylcyclopent-3-en-1-yl) but-2-en-1-ol, 1-[(2E,5E,9E)-2,6,10-trimethylcyclododeca-2,5,9-trien-1-yl]ethanone, 2-ethoxy-2,6,6-trimethyl-9-methylene-bicyclo[3.3.1]nonane, 4-(1-ethoxyvinyl)-3,3,5,5-tetramethyl-cyclohexanone, 1-(1,1,2,4,4,7-hexamethyltetralin-6-yl)ethanone and mixtures thereof;
and
at least one substance of group (C) in a total amount of between in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (C) consists of compounds having an earthy and/or sweet odor impression selected from the group consisting of chromen-2-one, 2-ethyl-3-hydroxy-pyran-4-one, 3-ethoxy-4-hydroxy-benzaldehyde, 4-methoxybenzaldehyde, 3-hydroxy-2-methyl-4h-pyran-4-one, 4-hydroxy-3-methoxy-benzaldehyde, (4-formyl-2-methoxy-phenyl) 2-methylpropanoate and mixtures thereof;
and
at least one essential oil and/or essence and/or absolue selected from the group consisting of bergamot oil, ginger root extract, lavender oil, vetiver oil, eucalyptus oil, grapefruit oil, jasmin absolue, lime oil, orange flower absolue, orange oil, rose absolue, sandalwood oil, tonka bean absolue,
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of allyl 3-cyclohexylpropanoate, 5-hexyltetrahydrofuran-2-one, methoxyphenylbutanone, (2E)-3,7-Dimethylocta-2,6-dienal, 5-heptyltetrahydrofuran-2-one, hexyl acetate, isopropyl tetradeconate, 6,6-dimethoxy-2,5,5-trimethylhex-2-ene, ethyl-3-oxobutanoate, ethyl-2-methylpentanoate, 5-hexyldihydro-4-methylfuran-2(3H)-one, 2-tert-butylcyclohexyl acetate, diethyl 2-methylenebutanedioate, 2-bicyclo[2.2.1]hept-5-enyl acetate, ethyl 3-oxobutanoate, linolal, mixture of diethyl (E/Z)-2-methylbut-2-enedioate and diethyl 2-methylenebutanedioate, mixture of (5-isopropenyl-2-methyl-cyclohex-2-en-1-yl) acetate and (5-isopropenyl-2-methyl-cyclohexyl) acetate, ethyl tetrahydrofuran-2-carboxylate, decanal, ethyl rac-(2r,3r)-3-methyl-3-phenyl-oxirane-2-carboxylate, allyl hexanoate, (1,1-dimethyl-2-phenyl-ethyl) acetate, 5-octyltetrahydrofuran-2-one, ethyl butanoate, ethyl 2-methylbutanoate, 3-methylbutyl acetate, ethyl 2-(2-methyl-1,3-dioxolan-2-yl)acetate, 2,4-dimethyl-4,4a,5,9b-tetrahydoindeno[1,2-d][1,3]dioxine, 2-methyl-4-propyl-1,3-oxathiane, 2,4,6-trimethyl-4-phenyl-1,3-dioxane, allyl heptanoate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 7-methyl-1,5-benzodioxepin-3-one, 5-isopropenyl-2-methyl-cyclohex-2-en-1-one, 2,4,4,7-tetramethyloct-6-en-3-one, 3a,4,5,6,7,7a-hexahydro-4,7-methanoinden-6-yl acetate, (e)-4-methyldec-3-en-5-oL and mixtures thereof.

Yet another preferred embodiment relates to the use of a fragrance composition for conveying a positive mood, preferably a fresh mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-trimethyl-benzenepropanol, 3-(4-ethylphenyl)-2,2-dimethylpropanal, (2E)-3,7-dimethylocta-2,6-dien-1-ol, methyl-2-[rac-(1S,2S)-3-oxo-2-pentyl-cyclopentyl]acetate, 3-(1,3-benzodioxol-5-yl)-2-methyl-propanal, (+)-3-(4-isopropylcyclohexen-1-yl)propanal, 1-phenylethyl acetate, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 3,7-dimethylocta-1,6-dien-3-ol, 4-(propan-2-yl)cyclohexyl]methanol, 4-(4-methylpent-3-enyl)cyclohex-3-ene-1-carbaldehyde, 3-(4-methoxyphenyl)-2-methyl-propanal and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-tetramethyldodecahydronaphtho[2,1-b]furan, [2-[1-(3,3-dimethylcyclohexyl)ethoxy]-2-methyl-propyl]propanoate, 1-[(2E,5E,9E)-2,6,10-trimethylcyclododeca-2,5,9-trien-1-yl]ethanone, oxacycloheptadec-10-en-2-one, 2-ethoxy-2,6,6-trimethyl-9-methylene-bicyclo[3.3.1]nonane, 1-(1,1,2,4,4,7-hexamethyltetralin-6-yl)ethanone and mixtures thereof;
at least one essential oil and/or essence and/or absolue selected from the group consisting of bergamot oil, ginger root extract, lavender oil, vetiver oil, artemisia oil, hemp essence, lemon oil, cardamom oil, camphor oil, eucalyptus oil, grapefruit oil, jasmin absolue, lime oil, orange oil, rose absolue, sage oil and sandalwood oil,
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of allyl 3-cyclohexylpropanoate, (2E)-3,7-dimethylocta-2,6-dienal, 5-hexyltetrahydrofuran-2-one, methoxyphenylbutanone, 6,6-Dimethoxy-2,5,5-trimethylhex-2-ene, isopropyl tetradeconate, ethyl 3-oxobutanoate, ethyl-2-methylpentanoate, 2,4-dimethyl-4,4a,5,9b-tetrahydoindeno[1,2-d][1,3]dioxine, allyl heptanoate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 7-methyl-1,5-benzodioxepin-3-one, 5-isopropenyl-2-methyl-cyclohex-2-en-1-one, 2,4,4,7-tetramethyloct-6-en-3-one, methyl 2-nonynoate, (4-tert-butylcyclohexyl) acetate, (E)-4-methyldec-3-en-5-ol and mixtures thereof.

Another preferred embodiment of the present invention relates to the use of a fragrance composition for conveying a positive mood, preferably an energizing mood, in a subject, preferably a human, wherein the fragrance composition comprises or consists of:
at least one substance of group (A) in a total amount of between 0.01 and 70 wt.-%, preferably of between 0.2 and 35 wt.-%, especially preferably of between 0.5 and 35 wt.-%, moreover preferably of between 1 and 20 wt.-%,
wherein group (A) consists of compounds with a bloomy odor impression selected from the group consisting of 2,6-dimethyloct-7-en-2-ol, (6E)-3,7-dimethylnona-1,6-dien-3-ol, 3-(4-ethylphenyl)-2,2-dimethylpropanal, 3-(1,3-benzodioxol-5-yl)-2-methyl-propanal, methyl 2-[(7-hydroxy-3,7-dimethyloctylidene)amino]benzoate, 2,6-dimethylheptan-2-ol, 3,7-dimethylocta-1,6-dien-3-ol and mixtures thereof, and mixtures thereof,
and
optionally at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from 1,1,2,3,3,-pentamethyl-2,5,6,7-tetrahydroinden-4-one, 4,6,6,7,8,8-hexamethyl-1,3,4,7-tetrahydrocyclopenta[g]isochromene, oxacyclohexadecen-2-one (mixture of isomers), pentadecan-15-olide, [2-[1-(3,3-dimethylcyclohexyl)ethoxy]-2-methyl-propyl]propanoate, 1-[(2E,5E,9E)-2,6,10-trimethylcyclododeca-2,5,9-trien-1-yl]ethanone, 2,2,6,8-tetramethyltricyclo[5.3.1.03,8]undecan-3-ol, 1-(2,6,6,8-tetramethyl-9-tricyclo[5.3.1.01,5]undec-8-enyl)ethanone, and mixtures thereof;
and
optionally at least one substance of group (C) in a total amount of between 0.01 and 35 wt.-%, preferably of between 0.2 and 20 wt.-%, especially preferably of between 0.5 and 10 wt.-%,
wherein group (C) consists of compounds having an earthy and/or sweet odor impression selected from the group consisting of 2-ethyl-3-hydroxy-pyran-4-one, 3-ethoxy-4-hydroxy-benzaldehyde, 3,6-Dimethyl-3H-benzofuran-2-one, 1-methoxy-4-[(E)-prop-1-enyl]benzene, 3-hydroxy-2-methyl-4h-pyran-4-one, 4-hydroxy-3-methoxy-benzaldehyde, (4-formyl-2-methoxy-phenyl) 2-methylpropanoate, and mixtures thereof;
and
at least one essential oil and/or essence and/or absolue selected from the group consisting of bergamot oil, ginger root extract, artemisia oil, cinnamon bark oil, elemi oil, ginger oil, lemon oil, lemongrass oil, longonza oil, mandarin oil, black pepper oil, red berry extract, camphor oil, cardamom oil, cypress oil, eucalyptus oil, grapefruit oil, lime oil, olibanum oil, orange oil, patchouli essence, rose absolue, rosemary oil, thyme oil and tuberose absolue, and
at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of (2E)-3,7-Dimethylocta-2,6-dienal, 5-hexyltetrahydrofuran-2-one, methoxyphenylbutanone, 6,6-Dimethoxy-2,5,5-trimethylhex-2-ene, hexyl acetate, decanal, ethyl butanoate, 3-methylbutyl acetate, 2,4-dimethyl-4,4a,5,9b-tetrahydoindeno[1,2-d][1,3]dioxine, 2-methyl-4-propyl-1,3-oxathiane, allyl heptanoate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 7-methyl-1,5-benzodioxepin-3-one, 1-methyl-4-(4-methylpent-3-enyl)cyclohex-3-ene-1-carbaldehyde, 2,4-Dimethylcyclohex-3-ene-1-carbaldehyde, undec-10-enal, 5-isopropenyl-2-methyl-cyclohex-2-en-1-one, 2,4,4,7-tetramethyloct-6-en-3-one, 4-allyl-2-methoxy-phenol, 2,6-dimethylhept-5-enal, (E)-4-methyldec-3-en-5-ol and mixtures thereof.

One preferred embodiment of the present invention relates to the use of a fragrance composition for conveying a positive mood, preferably a joyful mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 7-hydroxy-3,7-dimethyl-octanal, (6E)-3,7-dimethylnona-1,6-dien-3-ol, beta,beta-3-Trimethyl-benzenepropanol, methyl-2-aminobenzoate, (E)-4-(2,6,6-trimethylcyclohex-2-en-1-yl)but-3-en-2-one, benzyl acetate, 3-(4-ethylphenyl)-2,2-dimethylpropanal, (2E)-3,7-dimethylocta-2,6-dien-1-ol, 3-(1,3-benzodioxol-5-yl)-2-methyl-propanal, [(Z)-hex-3-enyl]-2-hydroxybenzoate, (E)-4-(2,6,6-trimethylcyclohexen-1-yl)but-3-en-2-one, (+)-3-(4-Isopropylcyclohexen-1-yl)propanal, methyl 2-[(7-hydroxy-3,7-dimethyloctylidene)amino]benzoate, ethyl 2,6,6-trimethylcyclohexa-1,3-diene-1-carboxylate, 3-(3-isopropylphenyl)butanal, 2-(phenylmethylidene)octanal, (E)-1-(2,4,4-trimethylcyclohexen-1-yl)but-2-en-1-one, 3,7-dimethylocta-1,6-dien-3-ol, 2-methyl-4-phenyl-butan-2-ol, 2-methyl-5-phenylpentanol, 2,2,5-trimethyl-5-pentyl-cyclopentanone, 3-(4-methoxyphenyl)-2-methyl-propanal and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, Oxacyclohexadecen-2-one (mixture of isomers), Pentadecan-15-olide, (E)-3-methyl-4-(2,6,6-trimethylcyclohex-2-en-1-yl)but-3-en-2-one, 1-[2,3,8,8-tetramethyl-1,3,4,5,6,7-hexanhydronaphthalen-2-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, (E)-2-methyl-3-phenyl-prop-2-en-1-ol, 4-(1-ethoxyvinyl)-3,3,5,5-tetramethyl-cyclohexanone, (E)-3,4,5,6,6-pentamethylhept-3-en-2-one, 1-(1,1,2,4,4,7-hexamethyltetralin-6-yl)ethanone and mixtures thereof,
and
at least one substance of group (C) in a total amount of between 0.01 and 35 wt.-%, preferably of between 0.2 and 20 wt.-%, especially preferably of between 0.5 and 10 wt.-%,
wherein group (C) consists of compounds having an earthy and/or sweet odor impression selected from the group consisting chromen-2-one, 2-ethyl-3-hydroxy-pyran-4-one, 3-ethoxy-4-hydroxy-benzaldehyde, 2-Methyl-5-propyl-cyclohex-2-en-1-one, 3,6-Dimethyl-3H-benzofuran-2-one, 1,3-benzodioxole-5-carbaldehyde, 5-pentyltetrahydrofuran-2-one, 1-methoxy-4-[(e)-prop-1-enyl]benzene, 4-methoxybenzaldehyde, 4-hydroxy-2,5-dimethylfuran-3-one, 3-hydroxy-2-methyl-4h-pyran-4-one, 4-hydroxy-3-methoxy-benzaldehyde, (4-formyl-2-methoxy-phenyl) 2-methylpropanoate and mixtures thereof; and
at least one essential oil and/or essence and/or absolue selected from the group consisting of bergamot oil, lavender oil, vetiver oil, Artemisia oil, artichoke essence, elemi oil, ginger oil, lemon oil, lemongrass oil, mandarin oil, black pepper oil, red berry extract, clove essence, vanilla absolue, ylang ylang oil, benzaldehyde, cardamom oil, cypress oil, eucalyptus oil, geranium oil, grapefruit oil, jasmin absolue, lime oil, olibanum oil, range flower absolue, orange oil, rose absolue, rosemary oil, sage oil, sandalwood oil, thyme oil and tonka bean absolue;
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of allyl 3-cyclohexylpropanoate, (2E)-3,7-Dimethylocta-2,6-dienal, 5-hexyltetrahydrofuran-2-one, methoxyphenylbutanone, 5-heptyltetrahydrofuran-2-one, 6,6-Dimethoxy-2,5,5-trimethylhex-2-ene, hexyl acetate, isopropyl tetradeconate, 2-tert-Butylcyclohexyl acetate, 5-Hexyldihydro-4-methylfuran-2(3H)-one, ethyl-2-methylpentanoate, mixture of diethyl (E/Z)-2-methylbut-2-enedioate and diethyl 2-methylenebutanedioate, ethyl rac-(2R,3R)-3-methyl-3-phenyl-oxirane-2-carboxylate, allyl hexanoate, 3-methylbutyl acetate, 2,4-dimethyl-4,4a,5,9b-tetrahydoindeno[1,2-d][1,3]dioxine, allyl heptanoate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 7-methyl-1,5-benzodioxepin-3-one, Hex-5-enyl 2-m,ethylbutanoate, [(Z)-hex-3-enyl]acetate, (Z)-Hex-3-en-1-yl methyl carbonate, 5-isopropenyl-2-methyl-cyclohex-2-en-1-one, 2,4,4,7-tetramethyloct-6-en-3-one, 3a,4,5,6,7,7a-hexahydro-4,7-methanoinden-6-yl acetate, (E)-4-methyldec-3-en-5-ol of and mixtures thereof.

Yet another preferred embodiment relates to the use of a fragrance composition for conveying a positive mood, preferably a relaxing mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 0.5 and 20 wt.-%,
wherein group (A) consists of compounds with a bloomy odor impression selected from the group consisting of 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, beta,beta-3-Trimethyl-benzenepropanol, methyl-2-aminobenzoate, (E)-4-(2,6,6-trimethylcyclohex-2-en-1-yl)but-3-en-2-one, benzyl 2-hydroxybenzoate, methyl-2-[RAC-(1S,2S)-3-oxo-2-pentyl-cyclopentyl]acetate, (+)-3-(4-Isopropylcyclohexen-1-yl)propanal, (1,5-dimethyl-1-vinyl-hex-4-enyl)acetate, 2-phenylethanol, 4-Isopentylcyclohexanol, methyl 2-[(7-hydroxy-3,7-dimethyloctylidene)amino]benzoate, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 2,3-dihydro-1,1-dimethyl-1h-indene-ar-propanal, (2e)-2-methyl-6-methylene-octa-2,7-dien-1-ol, 5-hexyl-5-methyl-tetrahydrofuran-2-one, 4-(propan-2-yl)cyclohexyl]methanol, 3-methyl-5-phenylpentan-1-ol, 2-methyl-5-phenylpentanol, 3-(4-methoxyphenyl)-2-methyl-propanal and mixtures thereof,
and
optionally at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, 1,4-dioxacycloheptadecane-5,17-dione, 4,6,6,7,8,8-hexamethyl-1,3,4,7-tetrahydrocyclopenta[G]isochromene, Oxacyclohexadecen-2-one (mixture of isomers), Pentadecan-15-olide, 8-methoxy-2,6,6,8-tetramethyl-tricyclo[5.3.1.01,5]undecane, [2-[1-(3,3-dimethylcyclohexyl)ethoxy]-2-methyl-propyl]propanoate, (E)-3-methyl-4-(2,6,6-trimethylcyclohex-2-en-1-yl)but-3-en-2-one, (5Z)-3-methylcyclopentadec-5-en-1-one, (2E)-2-Ethyl-4-(2,2,3)-trimethylcyclopent-3-en-1-yl) but-2-en-1-ol, 1-[(2E,5E,9E)-2,6,10-trimethylcyclododeca-2,5,9-trien-1-yl]ethanone, oxacycloheptadec-10-en-2-one, 2-ethoxy-2,6,6-trimethyl-9-methylene-bicyclo[3.3.1]nonane, (E)-2-methyl-3-phenyl-prop-2-en-1-ol, rac-(E,2R,3R)-3-methyl-5-(2,2,3-trimethylcyclopent-3-en-1-yl)pent-4-en-2-ol, 4-(1,7,7-trimethyl-2-bucyclo[3.1.1]heptanyl)ccyclohexan-1-ol, 4-(1-ethoxyvinyl)-3,3,5,5-tetramethyl-cyclohexanone, 1-(2,6,6,8-tetramethyl-9-tricyclo[5.3.1.01,5]undec-8-enyl)ethanone, 1-(1,1,2,4,4,7-hexamethyltetralin-6-yl)ethanone, Oxacycloheptadec-10-en-2-one, DL Ambre 83 SMP N, Methyl-2,4-dihydroxy-3,6-dimethyl-benzoate, (4-tert-butylcyclohexyl) acetate and mixtures thereof;
and
at least one substance of group (C) in a total amount of between 0.01 and 99 wt.-%, preferably of between 0.01 and 35 wt.-%, preferably of between 0.2 and 20 wt.-%, especially preferably of between 0.5 and 10 wt.-%,
wherein group (C) consists of compounds having an earthy and/or sweet odor impression selected from the group consisting of chromen-2-one, 4-methoxybenzaldehyde, 3-hydroxy-2-methyl-4H-pyran-4-one, 4,6,6,7,8,8-hexamethyl-1,3,4,7-tetrahydrocyclopenta[G]isochromene, 4-hydroxy-3-methoxy-benzaldehyde, (4-formyl-2-methoxy-phenyl) 2-methylpropanoate, [2-[1-(3,3-dimethylcyclohexyl)ethoxy]-2-methyl-propyl]propanoate, (E)-4-(2,6,6-trimethylcyclohex-2-en-1-yl)but-3-en-2-one, pentadecan-15-olide and mixtures thereof;
and
at least one essential oil and/or essence and/or absolue selected from the group consisting of lavender oil, cedarwood oil, cypress oil, eucalyptus oil, geranium oil, lime oil, orange flower absolue, rose absolue, rosemary oil, sage oil and tonka bean absolue, and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of 5-heptyltetrahydrofuran-2-one, 5-hexyldihydro-4-methylfuran-2(3h)-one, ethyl 3-oxobutanoate, 5-octyltetrahydrofuran-2-one, ethyl 2-(2-methyl-1,3-dioxolan-2-yl)acetate, gamma-dodecalactone and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting, 11,11-trimethyl-8-methylene-bicyclo[7.2.0]undec-4-ene, methyl-2,4-dihydroxy-3,6-dimethyl-benzoate, 5-isopropenyl-2-methyl-cyclohex-2-en-1-one, 3a,4,5,6,7,7a-hexahydro-4,7-methanoinden-6-yl acetate, 2-ethoxy-4-(methoxymethyl)phenol, (4-tert-butylcyclohexyl) acetate, (E)-4-methyldec-3-en-5-ol and mixtures thereof.

One preferred embodiment of the present invention relates to the use of a fragrance composition for conveying a positive mood, preferably a self-confident mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 3-(4-tert-butylphenyl)propanal, 7-hydroxy-3,7-dimethyl-octanal, 3,7-dimethyloct-6-en-1-ol, 3-(4-isopropylphenyl)-2-methyl-propanal, beta,beta-3-Trimethyl-benzenepropanol, (E)-4-(2,6,6-trimethylcyclohex-2-en-1-yl)but-3-en-2-one, benzyl 2-hydroxybenzoate, (+)-3-(4-Isopropylcyclohexen-1-yl)propanal, 2-phenylethanol, ethyl 2,6,6-trimethylcyclohexa-1,3-diene-1-carboxylate, 5-hexyl-5-methyl-tetrahydrofuran-2-one and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 1,1,2,3,3,-pentamethyl-2,5,6,7-tetrahydroinden-4-one, 1,4-dioxacycloheptadecane-5,17-dione, Oxacyclohexadecen-2-one (mixture of isomers), Pentadecan-15-olide, [2-[1-(3,3-dimethylcyclohexyl)ethoxy]-2-methyl-propyl]propanoate, Oxacycloheptadec-10-en-2-one, rac-(E,2R,3R)-3-methyl-5-(2,2,3-trimethylcyclopent-3-en-1-yl)pent-4-en-2-ol and mixtures thereof;
and
at least one substance of group (C) in a total amount of between 0.01 and 70 wt.-%, preferably of between 0.2 and 50 wt.-%, especially preferably of between 0.5 and 35 wt.-%, especially preferably of between 1 and 20 wt.-%, wherein group (C) consists of compounds having an earthy and/or sweet odor impression selected from the group consisting of chromen-2-one, 4-methoxybenzaldehyde and mixtures thereof and mixtures thereof;
and
optionally at least one essential oil and/or essence and/or absolue selected from the group consisting of lavender oil, cedarwood oil, geranium oil, jasmin absolue and sage oil,
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of 5-heptyltetrahydrofuran-2-one, 5-Hexyldihydro-4-methylfuran-2(3H)-one, ethyl 2-(2-methyl-1,3-dioxolan-2-yl)acetate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of a compound having green and/or herbal odor impression selected from 4,11,11-trimethyl-8-methylene-bicyclo[7.2.0]undec-4-ene, 2-ethoxy-4-(methoxymethyl)phenol and mixtures thereof.

Another preferred embodiment relates to the use of a fragrance composition for conveying a positive mood, preferably well-being mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 7-hydroxy-3,7-dimethyl-octanal, beta,beta-3-Trimethyl-benzenepropanol, (E)-4-(2,6,6-trimethylcyclohex-2-en-1-yl)but-3-en-2-one, benzyl acetate, 3-(1,3-benzodioxol-5-yl)-2-methyl-propanal, (E)-4-(2,6,6-trimethylcyclohexen-1-yl)but-3-en-2-one, (+)-3-(4-Isopropylcyclohexen-1-yl)propanal, methyl 2-[(7-hydroxy-3,7-dimethyloctylidene)amino]benzoate, ethyl 2,6,6-trimethylcyclohexa-1,3-diene-1-carboxylate, 3-(3-isopropylphenyl)butanal, 2-(phenylmethylidene)octanal, 2-methyl-4-phenyl-butan-2-ol, 2-methyl-5-phenylpentanol, 3-(4-methoxyphenyl)-2-methyl-propanal and mixtures thereof, and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, Oxacyclohexadecen-2-one (mixture of isomers), Pentadecan-15-olide, (E)-3-methyl-4-(2,6,6-trimethylcyclohex-2-en-1-yl)but-3-en-2-one, 1-[2,3,8,8-tetramethyl-1,3,4,5,6,7-hexanhydronaphthalen-2-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, (E)-2-methyl-3-phenyl-prop-2-en-1-ol, 4-(1-ethoxyvinyl)-3,3,5,5-tetramethyl-cyclohexanone, (e)-3,4,5,6,6-pentamethylhept-3-en-2-one, 1-(1,1,2,4,4,7-hexamethyltetralin-6-yl)ethanone and mixtures thereof;
and
at least one substance of group (C) in a total amount of between 0.01 and 35 wt.-%, preferably of between 0.2 and 20 wt.-%, especially preferably of between 0.5 and 10 wt.-%,
wherein group (C) consists of compounds having an earthy and/or sweet odor impression selected from the group consisting of chromen-2-one, 2-ethyl-3-hydroxy-pyran-4-one, 3-ethoxy-4-hydroxy-benzaldehyde, 2-Methyl-5-propyl-cyclohex-2-en-1-one, 3,6-Dimethyl-3H-benzofuran-2-one, 1,3-benzodioxole-5-carbaldehyde, 5-pentyltetrahydrofuran-2-one, 1-methoxy-4-[(E)-prop-1-enyl]benzene, 4-methoxybenzaldehyde, 4-hydroxy-2,5-dimethylfuran-3-one, 3-hydroxy-2-methyl-4h-pyran-4-one, 4-hydroxy-3-methoxy-benzaldehyde, (4-formyl-2-methoxy-phenyl) 2-methylpropanoate and mixtures thereof;
and
at least one essential oil and/or essence and/or absolue selected from the group consisting of bergamot oil, lavender oil, vetiver oil, artichoke essence, elemi oil, ginger oil, lemongrass oil, mandarin oil, black pepper oil, red berry extract, vanilla absolue, ylang ylang oil, cardamom oil, eucalyptus oil, geranium oil, grapefruit oil, jasmin absolue, lime oil, olibanum oil, orange flower absolue, orange oil, rose absolue, rosemary oil, sage oil, sandalwood oil, thyme oil and tonka bean absolue.
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of allyl 3-cyclohexylpropanoate, (2E)-3,7-Dimethylocta-2,6-dienal, 5-hexyltetrahydrofuran-2-one, Methoxyphenylbutanone, 5-heptyltetrahydrofuran-2-one, 6,6-Dimethoxy-2,5,5-trimethylhex-2-ene, hexyl acetate, isopropyl tetradeconate, ethyl-2-methylpentanoate, ethyl rac-(2R,3R)-3-methyl-3-phenyl-oxirane-2-carboxylate, allyl hexanoate, 3-methylbutyl acetate, allyl heptanoate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 7-methyl-1,5-benzodioxepin-3-one, hex-5-enyl 2-methylbutanoate, (Z)-Hex-3-en-1-yl methyl carbonate, 5-isopropenyl-2-methyl-cyclohex-2-en-1-one, 3a,4,5,6,7,7a-hexahydro-4,7-methanoinden-6-yl acetate, (E)-4-methyldec-3-en-5-ol and mixtures thereof.

It is preferred in terms of the present invention, that the fragrance composition in terms of the present invention contains at least one substance of group (F) in a total amount of between 0.01 and 35 wt.-%, preferably of between 0.2 and 20 wt.-%, especially preferably of between 0.5 and 10 wt.-%,
wherein group (F) consists of compounds selected from the group consisting of cassis 345b type base without myrcene, citrorange base colipa, cocoa aromabase, cuir velours, dewberry type base 2019, DL ambre 84 BC, DL ambre d'or BC, DL lily cristal BC, DL miel essentiel BC, DL noir prunol BC, DL osmanthus BC, DL poivre pique BC, DL rouge groseille BC, DL tabac bourbon BC, DL tubeliane BC, galbex type base, lavandin grosso bm, lilial replacer FF new, nectarol type base, orange blossom 163 c type base, peach aromabase (buchu replaced), pear base, prunella type base, suederal It, Synambran®R 50% in IPM, tamarine type base BHT free, vetiverylia base, Vitessence® white lavender BC, Vitessence® basil myrcene free, Vitessence® cucumber, Vitessence® cuir, Vitessence® green tea base, Vitessence® jasmine sambac, Vitessence® riesling, white vanilla base.

Another preferred embodiment relates to the use according to the invention, wherein the fragrance composition additionally comprises at least one solvent.

Yet another preferred embodiment relates to the use according to the invention, wherein the at least one solvent is/are selected from the group consisting of dipropylene glycol (DPG), isopropylmyristate (IPM), ethanol (EtOH) and triethylcitrate (TEC).

Preferably, the at least one solvent is/are incorporated in an amount of from 0.1 wt.-% to 50 wt.-%, preferably of from 0.5 wt.-% to 30 wt.-% and especially preferably in an amount of from 1 wt.-% to 20 wt.-%, each dependent on the total weight of the fragrance composition.

One preferred embodiment relates to the use according to the invention, wherein the fragrance composition is contained in a perfumed product. Preferably, the term “perfumed” has to be understood such that the fragrance composition is contained in a sensorially effective amount (however, the products used according to the invention may optionally comprise one or more further sensorially effective ingredients).

Preferably the fragrance composition is contained in an amount sufficient to enhance the mood of a subject, preferably a human, more preferably, wherein the fragrance composition is contained in a total amount in the range of from 0.01 to 35 wt.-%, preferably of from 0.2 to 25 wt.-%, more preferably of from 0.3 to 20 wt.-%, based on the total weight of the perfumed product.

The term “effective amount” refers to the amount of the fragrance composition, which is needed to create the desired response in a subject, preferably a human. Most importantly, the user must be able to perceive the odor of the fragrance when administering the fragrance experience according to its typical usage instructions. Preferably, the fragrance composition will be used at a concentration as described above.

In connection with the present invention, “mood enhancing” effects may be and are preferably determined according to one, more or all testing procedures described in example 1 below, and, thus, are preferably selected from the group consisting of vocal analysis (prosody), semantic analysis (verbatim), psychometric scale (GEW), implicit response test (IRT) and emotional characterization (EmoChar™).

Yet another preferred embodiment relates to the use according to the invention, wherein the perfumed product is selected from the group consisting of fine fragrance, personal cosmetic, toiletry, and healthcare products such as dry and wet wipes, washes, baths, shampoos, gels, soaps, mousses, cleansing compositions, bath oils, other bath compositions that may be added to a bath, other wash compositions that may be used directly on the skin, leave-on personal care products include personal cosmetic, toiletry, and healthcare products such as sticks, balms, mousses, sprays, lotions, creams, gels, powders, oils, waxes, perfumes, other personal care compositions that may be applied to the skin and not rinsed off, or home or laundry care products such as fabric detergents, fabric softeners, linen sprays, air care products, diffusers, devices or other products that may be used in the home for the purpose of cleaning surfaces, fabrics or perfuming the air.

Both cleansing and leave-on personal care products are particularly useful in delivering the fragrances of the present invention. For example, a fragrance composition may be produced by blending the selected fragrance ingredients under ambient conditions until the final mixture is homogenous using equipment and methodology commonly known in the art. It is preferable to store the final fragrance mixture (composition) under ambient conditions for a few hours after mixing before using it as a component of e.g. a cleansing or leave-on personal care product. In order to improve the solubilization of the fragrance composition in aqueous personal care products, the sensory fragrance may be pre-blended with one or more of the nonionic surfactants.

Products according to the present invention have been shown to be particularly useful in the context of the present invention as they increase the amount of time the user is willing to spend using the product, which enhances the benefit.

According to one embodiment of the use according to invention, the fragrance composition according to the present invention comprises a total amount of less than 35 different substances, preferably less than 20.

The present invention is based on extensive testing of fragrance raw materials using psychological and behavioral parameters to classify the fragrance raw materials for the purpose of the present invention (cf. testing procedures described in example 1), namely fragrances that induce in subjects exposed to them positive moods and emotions, particularly an attractive, a protective, a fresh, an energizing, a joyful, a relaxing, a sensual, a self-confident or a well-being mood.

When following the features of the attached claims and the herein described embodiments of compositions according to the invention as well as the testing procedures described herein (cf. example 1), a skilled person can easily provide a plurality of different fragrance compositions for a use according to the invention.

One aspect of the present invention relates to a method for imparting a positive mood, preferably an attractive, protective, joyful, fresh, energizing, relaxing, sensual, self-confident and/or well-being mood, consisting of or comprising the steps of:
i) Providing a fragrance composition, wherein the fragrance composition comprises or consists of:
   at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (A) consists of compounds with a bloomy odor impression selected from the group consisting of 3-(4-tert-butylphenyl)propanal, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-Trimethyl-benzenepropanol, methyl-2-aminobenzoate, (E)-4-(2,6,6-trimethylcyclohex-2-en-1-yl)but-3-en-2-one, 3-(4-ethylphenyl)-2,2-dimethylpropanal, 3-(1,3-benzodioxol-5-yl)-2-methyl-propanal, (2E)-3,7-dimethylocta-2,6-dien-1-ol, methyl-2-[RAC-(1S,2S)-3-oxo-2-pentyl-cyclopentyl]acetate, 1-phenylethyl acetate, benzyl acetate, 1,3-benzodioxole-5-carbaldehyde, [(Z)-hex-3-enyl]-2-hydroxybenzoate, (1,5-dimethyl-1-vinyl-hex-4-enyl)acetate, 2-phenylethanol, 4-Isopentylcyclohexanol, benzyl 2-hydroxybenzoate, 4-cyclohexylbutan-2-ol, (E)-1-(2,6,6-trimethylcyclohexan-1,3-dien-1-yl)but-2-en-1-one, (2E)-3,7-dimethylocta-2,6-dienyl]acetate, (E)-4-(2,6,6-trimethylcyclohexen-1-yl)but-3-en-2-one, (+)-3-(4-Isopropylcyclohexen-1-yl)propanal, 2,6-dimethyloct-7-en-2-ol 7-methyl-1,5-benzodioxepin-3-one, 1-methyl-4-(4-methylpent-3-enyl)cyclohex-3-ene-1-carbaldehyde, (E)-1-(2,6,6-trimethylcyclohex-2-en-1-yl)but-2-en-1-one, 2-isobutyl-4-methyl-tetrahydropyran-4-ol, [(2E)-3,7-dimethylocta-2,6-dienyl]acetate, indole, (1E)-1-(2,6,6-trimethylcyclohex-2-en-1-yl)hepta-1,6-dien-3-one, methyl 2-[(7-hydroxy-3,7-dimehyloctylidene)amino]benzoate, rac-(e)-1-[rac-(1r,2s)-2,6,6-trimethylcyclohex-3-en-1-yl]but-2-en-1-one, 2-methyl-1-phenyl-propan-2-ol, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 2,3-dihydro-1,1-dimethyl-1h-indene-ar-propanal, (E)-1-(2,4,4-trimethylcyclohexen-1-yl)but-2-en-1-one, (2E)-2-methyl-6-methylene-octa-2,7-dien-1-ol, 5-hexyl-5-methyl-tetrahydrofuran-2-one, 3,7-dimethylocta-1,6-dien-3-ol, 4-(propan-2-yl)cyclohexyl]methanol, 1-naphthalen-2-ylethanone, 2-ethoxynaphtalene, 2-methoxynaphtalene, 3-methyl-2-benzofuran-1-yl)ethanone, 2-phenoxyethyl 2-methylpropanoate, 3-methyl-5-phenylpentan-1-ol, 2-methyl-4-phenyl-butan-2-ol, (2,2,2-trichloro-1-phenyl-ethyl) acetate, 2-methyl-5-phenylpentanol, rac-(2r,4s)-4-methyl-2-(2-methylprop-1-enyl)tetrahydropyran, 2,2,5-trimethyl-5-pentyl-cyclopentanone, 3-(4-methoxyphenyl)-2-methyl-propanal, (6E)-3,7,11-trimethyldodeca-1,6,10-trien-3-ol, (6Z)-3,7,11-trimethyldodeca-1,6,10-trien-3-ol and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-tetramethyldodecahydronaphtho[2,1-b]furan, 1,1,2,3,3,-pentamethyl-2,5,6,7-tetrahydroinden-4-one, 8-methoxy-2,6,6,8-tetramethyl-tricyclo[5.3.1.01,5]undecane, (E)-3-methyl-4-(2,6,6-trimethylcyclohex-2-en-1-yl)but-3-en-2-one, (2E)-2-Ethyl-4-(2,2,3)-trimethylcyclopent-3-en-1-yl)but-2-en-1-ol, 1-[(2E,5E,9E)-2,6,10-trimethylcyclododeca-2,5,9-trien-1-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, 1-[2,3,8,8-tetramethyl-1,3,4,5,6,7-hexanhydronaphthalen-2-yl]ethanone, (Ethoxymethoxy)cyclododecane, dioxol-4H-4A,9-methanoazuleno(5,6-D)-octahydro-2-propyl-2,5,8,8,9A-pentamethyl-1,3, (E/Z)-4-Ethyloct-2/3-enenitrile, 3,4-dimethylbenzonitrile, (2E)-3-phenylprop-2-en-1-ol, 2,3,6-trimethylcyclohexanol, oxacycloheptadec-10-en-2-one, (4aR,5R,7aS,9R)-octahydro-2,2,5,8,8,9a-hexamethyl-4H-4a,9-methanoazuleno(5,6-d)-1,3-dioxole, 2-ethoxy-2,6,6-trimethyl-9-methylene-bicyclo[3.3.1]nonane, (E)-2-methyl-3-phenyl-prop-2-en-1-ol, rac-(E,2R,3R)-3-methyl-5-(2,2,3-trimethylcyclopent-3-en-1-yl)pent-4-en-2-ol, 4-(1,7,7-trimethyl-2-bucyclo[3.1.1]heptanyl)ccyclohexan-1-ol, 6-butan-2-ylquinoline, 4-(1-ethoxyvinyl)-3,3,5,5-tetramethyl-cyclohexanone, (E)-3,4,5,6,6-pentamethylhept-3-en-2-one, 2-(4-methylcyclohex-3-en-1-yl)propan-2-ol, 1-(2,6,6,8-tetramethyl-9-tricyclo[5.3.1.01,5]undec-8-enyl)ethanone, rac-(3′R,6′R,9′R)-2′,2′,8′,8′-tetramethylspiro[1,3-dioxolane-2,10′-octahydro-1h-2,4a-methanonapthalene], 1-(1,1,2,4,4,7-hexamethyltetralin-6-yl)ethanone, Oxacycloheptadec-10-en-2-one, DL Ambre 83 SMP N, methyl-2,4-dihydroxy-3,6-dimethyl-benzoate, (4-tert-butylcyclohexyl) acetate and mixtures thereof;
and
at least one substance of group (C) in a total amount of between 0.01 and 70 wt.-%, preferably of between 0.2 and 50 wt.-%, especially preferably of between 0.5 and 35 wt.-%, moreover preferably of between 1 and 20 wt.-% wherein group (C) consists of compounds having an earthy and/or sweet odor impression selected from the group consisting of chromen-2-one, 2-ethyl-3-hydroxy-pyran-4-one, (5Z)-3-methylcyclopentadec-5-en-1-one, 3-ethoxy-4-hydroxy-benzaldehyde, 1,4-dioxacycloheptadecane-5,17-dione, 4,6,6,7,8,8-hexamethyl-1,3,4,7-tetrahydrocyclopenta[G]isochromene, oxacyclohexadecen-2-one (mixture of isomers), Pentadecan-15-olide, [2-[1-(3,3-dimethylcyclohexyl)ethoxy]-2-methyl-propyl]propanoate, 3,6-Dimethyl-3H-benzofuran-2-one, (5Z)-3-methylcyclopentadec-5-en-1-one, 4,6,6,7,8,8-hexamethyl-1,3,4,7-tetrahydrocyclopenta[G]isochromene, 2-methyl-5-propyl-cyclohex-2-en-1-one, 1,3-benzodioxole-5-carbaldehyde, 1-pyrazin-2-ylethanone, 5-pentyltetrahydrofuran-2-one, 1-methoxy-4-[(e)-prop-1-enyl]benzene, 4-methoxybenzaldehyde, 4-hydroxy-2,5-dimethylfuran-3-one, 3-hydroxy-2-methyl-4h-pyran-4-one, 2-(4-methylthiazol-5-yl)ethanol, 4-hydroxy-3-methoxy-benzaldehyde, (4-formyl-2-methoxy-phenyl)-2-methylpropanoate, benzaldehyde and mixtures thereof,
and
at least one essential oil and/or essence and/or absolue selected from the group consisting of lavender oil, bergamot oil, ginger root extract, vetiver oil, lemon oil, artemisia oil, cinnamon bark oil, elemi oil, ginger oil, lemongrass oil, longoza oil, mandarin oil, pepper black oil, vanilla absolue, red berry extract, bay oil, cedarwood oil, ylang ylang oil, hemp oil, pink pepper leaf oil, amyris oil, cardamom oil, carrot seed oil, cypress oil, davana oil, geranium oil, grapefruit oil, lime oil, olibanum oil, palmarosa oil, sage oil, sandalwood oil, thyme oil, artichoke essence, asparagus essence, cauliflower essence, hemp essence, leek essence, clove essence, passionfruit essence, amber base, red apple essence, benzaldehyde, Styrax tonkinensis essence, camphor oil, eucalyptus oil, jasmin oil, jasmin absolue, orange oil, rosemary oil, galbanum essence, orange flower absolue, patchouli essence, rose absolue, tonka bean absolue, tuberose absolue, rose bourboniana absolute;
ii) contacting the fragrance composition with the subject, preferably a human, in an amount sufficient to enhance the mood of the subject/human.

The expression “contacting” in terms of the present invention describes any direct or indirect action, which is suitable to bring the subject, preferably a human, in contact with the fragrance composition in such a manner, that the subject, preferably a human, perceives the olfactory active substances in the fragrance composition. “Contacting” in terms of the present invention, thus, may be understood broadly and can for example be done by spraying, rubbing, massaging, creaming the fragrance composition to the hair, skin or nails of the subject, but also by applying the fragrance composition to items the subject is interacting with or by contacting the air surrounding the subject, preferably a human.

Further examples become apparent in view of the herein described products according to the invention.

A preferred embodiment relates to the method according to the invention, wherein the fragrance composition additionally comprises
at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of 2-bicyclo[2.2.1]hept-5-enyl acetate, ethyl 3-oxobutanoate, linolal, mixture of diethyl (E/Z)-2-methylbut-2-enedioate and diethyl 2-methylenebutanedioate, mixture of (5-isopropenyl-2-methyl-cyclohex-2-en-1-yl) acetate and (5-isopropenyl-2-methyl-cyclohexyl) acetate, ethyl tetrahydrofuran-2-carboxylate, decanal, ethyl rac-(2r,3r)-3-methyl-3-phenyl-oxirane-2-carboxylate, allyl hexanoate, (1,1-dimethyl-2-phenyl-ethyl) acetate, 5-octyltetrahydrofuran-2-one, ethyl butanoate, ethyl 2-methylbutanoate, 3-methylbutyl acetate, ethyl 2-(2-methyl-1,3-dioxolan-2-yl)acetate, 2,4-dimethyl-4,4a,5,9b-tetrahydoindeno[1,2-d][1,3]dioxine, 2-methyl-4-propyl-1,3-oxathiane, 2,4,6-trimethyl-4-phenyl-1,3-dioxane, allyl heptanoate, gamma-dodecalactone and mixtures thereof.

Yet another preferred embodiment of the method according to the invention relates to a method, wherein the fragrance composition additionally comprises at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of allyl (cyclohexyloxy)acetate, 3,3,5-trimethylcyclohexyl acetate, 1-methyl-4-(4-methylpent-3-enyl)cyclohex-3-ene-1-carbaldehyde, undec-10-enal, 5-isopropenyl-2-methyl-cyclohex-2-en-1-one, 2,4,4,7-tetramethyloct-6-en-3-one, 4-allyl-2-methoxy-phenol, 3a,4,5,6,7,7a-hexahydro-4,7-methanoinden-6-yl acetate, 2,6-dimethylhept-5-enal, 2-ethoxy-4-(methoxymethyl)phenol, methyl 2-nonynoate, (4-tert-butylcyclohexyl) acetate, 5-methylheptan-3-one oxime, (E)-4-methyldec-3-en-5-ol, and mixtures thereof.

A preferable embodiment of the present invention relates to a method for imparting a positive mood, preferably a protective mood, in a subject, preferably a human, consisting of or comprising the steps of:
i) Providing a fragrance composition, wherein the fragrance composition comprises or consists of:
   Optionally at least one substance of group (A) in a total amount of between 0.01 and 70 wt.-%, preferably of between 0.2 and 50 wt.-%, especially preferably of between 0.5 and 20 wt.-%, moreover preferably of between 1 and 20 wt.-%,
   wherein group (A) consists of compounds with a bloomy odor impression selected from 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-trimethyl-benzenepropanol, methyl-2-aminobenzoate, (E)-4-(2,6,6-trimethylcyclohex-2-en-1-yl)but-3-en-2-one, methyl 2-[(7-hydroxy-3,7-dimethyloctylidene)amino]benzoate, 2,6-dimethylheptan-2-ol, 5-hexyl-5-methyl-tetrahydrofuran-2-one, 3,7-dimethylocta-1,6-dien-3-ol, 4-(propan-2-yl)cyclohexyl]methanol, 3-(4-methoxyphenyl)-2-methyl-propanal and mixtures thereof, preferably selected from 2,6-dimethylheptan-2-ol,
   and
   at least one substance of group (B) in a total amount of between 0.01 and 50 wt.-%, preferably of between 0.2 and 35 wt.-%, especially preferably of between 0.5 and 20 wt.-%,
   wherein group (B) consists of compounds with a woody and/or balsam odor impression, selected from the group consisting of 11,1,2,3,3,-pentamethyl-2,5,6,7-tetrahydroinden-4-one, 4,6,6,7,8,8-hexamethyl-1,3,4,7-tetrahydrocyclopenta[g]isochromene, oxacyclohexadecen-2-one (mixture of isomers), pentadecan-15-olide, [2-[1-(3,3-dimethylcyclohexyl)ethoxy]-2-methyl-propyl]propanoate, 1-[(2E,5E,9E)-2,6,10-trimethylcyclododeca-2,5,9-trien-1-yl]ethanone, 2,2,6,8-tetramethyltricyclo[5.3.1.03,8]undecan-3-ol, 1-(2,6,6,8-tetramethyl-9-tricyclo[5.3.1.01,5]undec-8-enyl)ethanone and mixtures thereof;
   and
   at least one substance of group (C) in a total amount of between 0.01 and 99 wt.-%, preferably of between 0.01 and 35 wt.-%, preferably of between 0.2 and 20 wt.-%, especially preferably of between 0.5 and 10 wt.-%, wherein group (C) consists of compounds having an earthy and/or sweet odor impression selected from the group consisting 3-ethoxy-4-hydroxy-benzaldehyde, (4-formyl-2-methoxy-phenyl) 2-methylpropanoate and mixtures thereof;
   and
   at least one essential oil and/or essence and/or absolue selected from the group consisting of lavender oil, eucalyptus oil, geranium oil, rose absolue, sage oil and tonka bean absolue;
   optionally at least one substance of group (D) in a total amount of between 0.01 and 35 wt.-%, preferably of between 0.2 and 20 wt.-%, especially preferably of between 0.5 and 10 wt.-%,
   wherein group (D) consists of compounds with a fruity odor impression, selected from the group consisting of allyl 3-cyclohexylpropanoate, 5-hexyltetrahydrofuran-2-one, methoxyphenylbutanone, (2E)-3,7-Dimethylocta-2,6-dienal, 5-heptyltetrahydrofuran-2-one, hexyl acetate, isopropyl tetradeconate, 6,6-dimethoxy-2,5,5-trimethylhex-2-ene, ethyl-3-oxobutanoate, ethyl-2-methylpentanoate, 5-hexyldihydro-4-methylfuran-2(3H)-one, 2-tert-butylcyclohexyl acetate, diethyl 2-methylenebutanedioate, 2-bicyclo[2.2.1]hept-5-enyl acetate, ethyl 3-oxobutanoate, linolal, mixture of diethyl (E/Z)-2-methylbut-2-enedioate and diethyl 2-methylenebutanedioate, mixture of (5-isopropenyl-2-methyl-cyclohex-2-en-1-yl) acetate and (5-isopropenyl-2-methyl-cyclohexyl) acetate, ethyl tetrahydrofuran-2-carboxylate, decanal, ethyl rac-(2r,3r)-3-methyl-3-phenyl-oxirane-2-carboxylate, allyl hexanoate, (1,1-dimethyl-2-phenyl-ethyl) acetate, 5-octyltetrahydrofuran-2-one, ethyl butanoate, ethyl 2-methylbutanoate, 3-methylbutyl acetate, ethyl 2-(2-methyl-1,3-dioxolan-2-yl)acetate, 2,4-dimethyl-4,4a,5,9b-tetrahydoindeno[1,2-d][1,3]dioxine, 2-methyl-4-propyl-1,3-oxathiane, 2,4,6-trimethyl-4-phenyl-1,3-dioxane, allyl heptanoate and mixtures thereof,
   and
   optionally at least one substance of group (E) in a total amount of between 0.01 and 35 wt.-%, preferably of between 0.2 and 20 wt.-%, especially preferably of between 0.5 and 10 wt.-%,
   wherein group (E) consists of compounds having green and/or herbal odor impression selected from the group consisting of allyl-2isopentyloxyacetate, allyl(cyclohexyloxy)acetate, 1-(5,5-dimethylcyclohexen-1-yl)pent-4-en-1-one, 2,4-dimethylcyclohex-3-ene-1-carbaldehyde, (Z)-hex-3-en-1-yl methyl carbonate, hex-5-enyl 2-methylbutanoate, (Z)-hex-3-en-1-ol, [(Z)-hex-3-enyl]acetate, 4,11,11-trimethyl-8-methylene-bicyclo[7.2.0]undec-4-ene, methyl-2,4-dihydroxy-3,6-dimethyl-benzoate, allyl (cyclohexyloxy)acetate, 3,3,5-trimethylcyclohexyl acetate, 1-methyl-4-(4-methylpent-3-enyl)cyclohex-3-ene-1-carbaldehyde, undec-10-enal, 5-isopropenyl-2-methyl-cyclohex-2-en-1-one, 2,4,4,7-tetramethyloct-6-en-3-one, 4-allyl-2-methoxy-phenol, 3a,4,5,6,7,7a-hexahydro-4,7-methanoinden-6-yl acetate, 2,6-dimethylhept-5-enal, 2-ethoxy-4-(methoxymethyl)phenol, methyl 2-nonynoate, (4-tert-butylcyclohexyl) acetate, 5-methylheptan-3-one oxime, (E)-4-methyldec-3-en-5-ol, and mixtures thereof.
ii) contacting the fragrance composition with the subject, preferably a human, in an amount sufficient to enhance the mood of the subject/human.

One preferred embodiment of the present invention relates to a method for imparting a positive mood, preferably a sensual mood, in a subject, preferably a human, consisting of or comprising the steps of:
i) Providing a fragrance composition, wherein the fragrance composition comprises or consists of:
   at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (A) consists of compounds with a bloomy odor impression selected from the group consisting of 2,6-dimethyloct-7-en-2-ol, beta,beta-3-Trimethyl-benzenepropanol and mixtures thereof,
   and
   at least one substance of group (B) in a total amount of between 0.01 and 50 wt.-%, preferably of between 0.2 and 35 wt.-%, especially preferably of between 0.5 and 20 wt.-%,
   wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, 1,4-dioxacycloheptadecane-5,17-dione, 8-methoxy-2,6,6,8-tetramethyl-tricyclo[5.3.1.01,5]undecane, 1-[2,3,8,8-tetramethyl-1,3,4,5,6,7-hexanhydronaphthalen-2-yl]ethanone, 2,2,6,8-tetramethyltricyclo[5.3.1.03,8]undecan-3-ol, 2-ETHOXY-2,6,6-trimethyl-9-methylene-bicyclo[3.3.1]nonane, 4-(1-ethoxyvinyl)-3,3,5,5-tetramethyl-cyclohexanone, 1-(2,6,6,8-tetramethyl-9-tricyclo[5.3.1.01,5]undec-8-enyl)ethanone, 1-(1,1,2,4,4,7-hexamethyltetralin-6-yl)ethanone and mixtures thereof, and mixtures thereof;
   and
   at least one substance of group (C) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (C) consists of compounds having an earthy and/or sweet odor impression selected from the group consisting of 3,6-dimethyl-3H-benzofuran-2-one, 4-hydroxy-3-methoxy-benzaldehyde, 1,4-dioxacycloheptadecane-5,17-dione, (4-formyl-2-methoxy-phenyl) 2-methylpropanoate and mixtures thereof;
   and
   at least one essential oil and/or essence and/or absolue selected from the group consisting of vetiver oil, vanilla absolue, jasmin absolue, patchouli oil, rose absolue and sandalwood oil;
   and
   optionally at least one substance of group (D) in a total amount of between 0.01 and 35 wt.-%, preferably of between 0.2 and 20 wt.-%, especially preferably of between 0.5 and 10 wt.-%,
   wherein group (D) consists of compounds with a fruity odor impression, selected from the group consisting of allyl 3-cyclohexylpropanoate, 5-hexyltetrahydrofuran-2-one, methoxyphenylbutanone, (2E)-3,7-Dimethylocta-2,6-dienal, 5-heptyltetrahydrofuran-2-one, hexyl acetate, isopropyl tetradeconate, 6,6-dimethoxy-2,5,5-trimethylhex-2-ene, ethyl-3-oxobutanoate, ethyl-2-methylpentanoate, 5-hexyldihydro-4-methylfuran-2(3H)-one, 2-tert-butylcyclohexyl acetate, diethyl 2-methylenebutanedioate, 2-bicyclo[2.2.1]hept-5-enyl acetate, ethyl 3-oxobutanoate, linolal, mixture of diethyl (E/Z)-2-methylbut-2-enedioate and diethyl 2-methylenebutanedioate, mixture of (5-isopropenyl-2-methyl-cyclohex-2-en-1-yl) acetate and (5-isopropenyl-2-methyl-cyclohexyl) acetate, ethyl tetrahydrofuran-2-carboxylate, decanal, ethyl rac-(2r,3r)-3-methyl-3-phenyl-oxirane-2-carboxylate, allyl hexanoate, (1,1-dimethyl-2-phenyl-ethyl) acetate, 5-octyltetrahydrofuran-2-one, ethyl butanoate, ethyl 2-methylbutanoate, 3-methylbutyl acetate, ethyl 2-(2-methyl-1,3-dioxolan-2-yl)acetate, 2,4-dimethyl-4,4a,5,9b-tetrahydoindeno[1,2-d][1,3]dioxine, 2-methyl-4-propyl-1,3-oxathiane, 2,4,6-trimethyl-4-phenyl-1,3-dioxane, allyl heptanoate and mixtures thereof;
   and
   optionally at least one substance of group (E) in a total amount of between 0.01 and 35 wt.-%, preferably of between 0.2 and 20 wt.-%, especially preferably of between 0.5 and 10 wt.-%,
   wherein group (E) consists of compounds having green and/or herbal odor impression selected from the group consisting of allyl-2isopentyloxyacetate, allyl(cyclohexyloxy)acetate, 1-(5,5-dimethylcyclohexen-1-yl)pent-4-en-1-one, 2,4-dimethylcyclohex-3-ene-1-carbaldehyde, (Z)-hex-3-en-1-yl methyl carbonate, hex-5-enyl 2-methylbutanoate, (Z)-hex-3-en-1-ol, [(Z)-hex-3-enyl]acetate, 4,11,11-trimethyl-8-methylene-bicyclo[7.2.0]undec-4-ene, methyl-2,4-dihydroxy-3,6-dimethyl-benzoate, allyl (cyclohexyloxy)acetate, 3,3,5-trimethylcyclohexyl acetate, 1-methyl-4-(4-methylpent-3-enyl)cyclohex-3-ene-1-carbaldehyde, undec-10-enal, 5-isopropenyl-2-methyl-cyclohex-2-en-1-one, 2,4,4,7-tetramethyloct-6-en-3-one, 4-allyl-2-methoxy-phenol, 3a,4,5,6,7,7a-hexahydro-4,7-methanoinden-6-yl acetate, 2,6-dimethylhept-5-enal, 2-ethoxy-4-(methoxymethyl)phenol, methyl 2-nonynoate, (4-tert-butylcyclohexyl) acetate, 5-methylheptan-3-one oxime, (E)-4-methyldec-3-en-5-ol, and mixtures thereof.
ii) contacting the fragrance composition with the subject, preferably a human, in an amount sufficient to enhance the mood of the subject/human.

One preferred embodiment of the present invention relates to the method according to the invention for imparting a positive mood, preferably an attractive mood, in a subject, preferably a human, consisting of or comprising the steps of:
i) Providing a fragrance composition, wherein the fragrance composition comprises or consists of:
   at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (A) consists of compounds with a bloomy odor impression selected from the group consisting of -(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-trimethyl-benzenepropanol, methyl-2-aminobenzoate, (E)-4-(2,6,6-trimethylcyclohex-2-en-1-yl)but-3-en-2-one, methyl 2-[(7-hydroxy-3,7-dimethyloctylidene)amino]benzoate, 2,6-dimethylheptan-2-ol, 5-hexyl-5-methyl-tetrahydrofuran-2-one, 3,7-dimethylocta-1,6-dien-3-ol, 4-(propan-2-yl)cyclohexyl]methanol, 3-(4-methoxyphenyl)-2-methyl-propanal and mixtures thereof,
   and
   at least one substance of group (B) in a total amount of between 0.01 and 50 wt.-%, preferably of between 0.2 and 35 wt.-%, especially preferably of between 0.5 and 20 wt.-%,
   wherein group (B) consists of compounds with a woody and/or balsam odor impression, selected from the group consisting of 3a,6,6,9a-tetramethyldodecahydronaphtho[2,1-b]furan, 1,1,2,3,3,-pentamethyl-2,5,6,7-tetrahydroinden-4-one, 1,4-dioxacycloheptadecane-5,17-dione, 4,6,6,7,8,8-hexamethyl-1,3,4,7-tetrahydrocyclopenta[g]isochromene, oxacyclohexadecen-2-one (mixture of isomers), pentadecan-15-olide 8-methoxy-2,6,6,8-tetramethyl-tricyclo[5.3.1.01,5]undecane, [2-[1-(3,3-dimethylcyclohexyl)ethoxy]-2-methyl-propyl]propanoate, (E)-3-methyl-4-(2,6,6-trimethylcyclohex-2-en-1-yl)but-3-en-2-one, (5Z)-3-methylcyclopentadec-5-en-1-one, (2E)-2-ethyl-4-(2,2,3)-trimethylcyclopent-3-en-1-yl) but-2-en-1-ol, 1-[(2E,5E,9E)-2,6,10-trimethylcyclododeca-2,5,9-trien-1-yl]ethanone, 2-ethoxy-2,6,6-trimethyl-9-methylene-bicyclo[3.3.1]nonane, 4-(1-ethoxyvinyl)-3,3,5,5-tetramethyl-cyclohexanone, 1-(1,1,2,4,4,7-hexamethyltetralin-6-yl)ethanone and mixtures thereof;
   and
   at least one substance of group (C) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (C) consists of compounds having an earthy and/or sweet odor impression selected from the group consisting of chromen-2-one, 2-ethyl-3-hydroxy-pyran-4-one, 3-ethoxy-4-hydroxy-benzaldehyde, 4-methoxybenzaldehyde, 3-hydroxy-2-methyl-4h-pyran-4-one, 4-hydroxy-3-methoxy-benzaldehyde, (4-formyl-2-methoxy-phenyl) 2-methylpropanoate and mixtures thereof,
   and
   optionally at least one essential oil and/or essence and/or absolue selected from the group consisting of bergamot oil, ginger root extract, lavender oil, vetiver oil, eucalyptus oil, grapefruit oil, jasmin absolue, lime oil, orange flower absolue, orange oil, rose absolue, sandalwood oil, tonka bean absolue,
   and
   optionally at least one substance of group (D) in a total amount of between 0.01 and 35 wt.-%, preferably of between 0.2 and 20 wt.-%, especially preferably of between 0.5 and 10 wt.-%,
   wherein group (D) consists of compounds with a fruity odor impression, selected from the group consisting of allyl 3-cyclohexylpropanoate, 5-hexyltetrahydrofuran-2-one, methoxyphenylbutanone, (2E)-3,7-Dimethylocta-2,6-dienal, 5-heptyltetrahydrofuran-2-one, hexyl acetate, isopropyl tetradeconate, 6,6-dimethoxy-2,5,5-trimethylhex-2-ene, ethyl-3-oxobutanoate, ethyl-2-methylpentanoate, 5-hexyldihydro-4-methylfuran-2(3H)-one, 2-tert-butylcyclohexyl acetate, diethyl 2-methylenebutanedioate, 2-bicyclo[2.2.1]hept-5-enyl acetate, ethyl 3-oxobutanoate, linolal, mixture of diethyl (E/Z)-2-methylbut-2-enedioate and diethyl 2-methylenebutanedioate, mixture of (5-isopropenyl-2-methyl-cyclohex-2-en-1-yl) acetate and (5-isopropenyl-2-methyl-cyclohexyl) acetate, ethyl tetrahydrofuran-2-carboxylate, decanal, ethyl rac-(2r,3r)-3-methyl-3-phenyl-oxirane-2-carboxylate, allyl hexanoate, (1,1-dimethyl-2-phenyl-ethyl) acetate, 5-octyltetrahydrofuran-2-one, ethyl butanoate, ethyl 2-methylbutanoate, 3-methylbutyl acetate, ethyl 2-(2-methyl-1,3-dioxolan-2-yl)acetate, 2,4-dimethyl-4,4a, 5,9b-tetrahydoindeno[1.2-d][1,3]dioxine, 2-methyl-4-propyl-1,3-oxathiane, 2,4,6-trimethyl-4-phenyl-1,3-dioxane, allyl heptanoate, and mixtures thereof;
   and
   optionally at least one substance of group (E) in a total amount of between 0.01 and 35 wt.-%, preferably of between 0.2 and 20 wt.-%, especially preferably of between 0.5 and 10 wt.-%,
   wherein group (E) consists of compounds having green and/or herbal odor impression selected from the group consisting of 7-methyl-1,5-benzodioxepin-3-one, 5-isopropenyl-2-methyl-cyclohex-2-en-1-one, 2,4,4,7-tetramethyloct-6-en-3-one, 3a,4,5,6,7,7a-hexahydro-4,7-methanoinden-6-yl acetate, (e)-4-methyldec-3-en-5-oL and mixtures thereof.
ii) contacting the fragrance composition with the subject, preferably a human, in an amount sufficient to enhance the mood of the subject/human.

Yet another preferred embodiment relates to the method according to the invention for imparting a positive mood, preferably a fresh mood, in a subject, preferably a human, consisting of or comprising the steps of:
i) Providing a fragrance composition, wherein the fragrance composition comprises or consists of:
   at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (A) consists of compounds with a bloomy odor impression selected from the group consisting of 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-trimethyl-benzenepropanol, 3-(4-ethylphenyl)-2,2-dimethylpropanal, (2E)-3,7-dimethylocta-2,6-dien-1-ol, methyl-2-[rac-(1S,2S)-3-oxo-2-pentyl-cyclopentyl]acetate, 3-(1,3-benzodioxol-5-yl)-2-methyl-propanal, (+)-3-(4-isopropylcyclohexen-1-yl)propanal, 1-phenylethyl acetate, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 3,7-dimethylocta-1,6-dien-3-ol, 4-(propan-2-yl)cyclohexyl]methanol, 4-(4-methylpent-3-enyl)cyclohex-3-ene-1-carbaldehyde, 3-(4-methoxyphenyl)-2-methyl-propanal and mixtures thereof,
   and
   at least one substance of group (B) in a total amount of between 0.01 and 50 wt.-%, preferably of between 0.2 and 35 wt.-%, especially preferably of between 0.5 and 20 wt.-%,
   wherein group (B) consists of compounds with a woody and/or balsam odor impression, selected from the group consisting of 3a,6,6,9a-tetramethyldodecahydronaphtho[2,1-b]furan, [2-[1-(3,3-dimethylcyclohexyl)ethoxy]-2-methyl-propyl]propanoate, 1-[(2E,5E,9E)-2,6,10-trimethylcyclododeca-2,5,9-trien-1-yl]ethanone, oxacycloheptadec-10-en-2-one, 2-ethoxy-2,6,6-trimethyl-9-methylene-bicyclo[3.3.1]nonane, 1-(1,1,2,4,4,7-hexamethyltetralin-6-yl)ethanone and mixtures thereof,
   and
   at least one essential oil and/or essence and/or absolue selected from the group consisting of bergamot oil, ginger root extract, lavender oil, vetiver oil, artemisia oil, hemp essence, lemon oil, cardamom oil, camphor oil, eucalyptus oil, grapefruit oil, jasmin absolue, lime oil, orange oil, rose absolue, sage oil and sandalwood oil.
   and
   optionally at least one substance of group (D) in a total amount of between 0.01 and 35 wt.-%, preferably of between 0.2 and 20 wt.-%, especially preferably of between 0.5 and 10 wt.-%,
   wherein group (D) consists of compounds with a fruity odor impression, selected from the group consisting of allyl 3-cyclohexylpropanoate, (2E)-3,7-dimethylocta-2,6-dienal, 5-hexyltetrahydrofuran-2-one, Methoxyphenylbutanone, 6,6-Dimethoxy-2,5,5-trimethylhex-2-ene, isopropyl tetradeconate, ethyl 3-oxobutanoate, ethyl-2-methylpentanoate, 2,4-dimethyl-4,4a,5,9b-tetrahydoindeno[1,2-d][1,3]dioxine, allyl heptanoate and mixtures thereof;
   and
   optionally at least one substance of group (E) in a total amount of between 0.01 and 35 wt.-%, preferably of between 0.2 and 20 wt.-%, especially preferably of between 0.5 and 10 wt.-%,
   wherein group (E) consists of compounds having green and/or herbal odor impression selected from the group consisting of 7-methyl-1,5-benzodioxepin-3-one, 5-isopropenyl-2-methyl-cyclohex-2-en-1-one, 2,4,4,7-tetramethyloct-6-en-3-one, methyl 2-nonynoate, (4-tert-butylcyclohexyl) acetate, (E)-4-methyldec-3-en-5-ol and mixtures thereof.
ii) contacting the fragrance composition with the subject, preferably a human, in an amount sufficient to enhance the mood of the subject/human.

Another preferred embodiment of the present invention relates to the method according to the invention for imparting a positive mood, preferably an energizing mood, in a subject, preferably a human, consisting of or comprising the steps of:
i) Providing a fragrance composition, wherein the fragrance composition comprises or consists of:
   at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (A) consists of compounds with a bloomy odor impression selected from the group consisting of 2,6-dimethyloct-7-en-2-ol, (6E)-3,7-dimethylnona-1,6-dien-3-ol, 3-(4-ethylphenyl)-2,2-dimethylpropanal, 3-(1,3-benzodioxol-5-yl)-2-methyl-propanal, methyl 2-[(7-hydroxy-3,7-dimethyloctylidene)amino]benzoate, 2,6-dimethylheptan-2-ol, 3,7-dimethylocta-1,6-dien-3-ol and mixtures thereof,
   and
   optionally at least one substance of group (B) in a total amount of between 0.01 and 50 wt.-%, preferably of between 0.2 and 35 wt.-%, especially preferably of between 0.5 and 20 wt.-%,
   wherein group (B) consists of compounds with a woody and/or balsam odor impression, selected from (4aR,5R,7aS,9R)-octahydro-2,2,5,8,8,9a-hexamethyl-4h-4a,9-methanoazuleno(5,6-d)-1,3-dioxole;
   and
   optionally at least one substance of group (C) in a total amount of between 0.01 and 35 wt.-%, preferably of between 0.2 and 20 wt.-%, especially preferably of between 0.5 and 10 wt.-%,
   wherein group (C) consists of compounds having an earthy and/or sweet odor impression selected from the group consisting of 2-ethyl-3-hydroxy-pyran-4-one, 3-ethoxy-4-hydroxy-benzaldehyde, 3,6-Dimethyl-3H-benzofuran-2-one, 1-methoxy-4-[(E)-prop-1-enyl]benzene, 3-hydroxy-2-methyl-4h-pyran-4-one, 4-hydroxy-3-methoxy-benzaldehyde, (4-formyl-2-methoxy-phenyl) 2-methylpropanoate and mixtures thereof;
   and at least one essential oil and/or essence and/or absolue selected from the group consisting of bergamot oil, ginger root extract, artemisia oil, cinnamon bark oil, elemi oil, ginger oil, lemon oil, lemongrass oil, longonza oil, mandarin oil, black pepper oil, red berry extract, camphor oil, cardamom oil, cypress oil, eucalyptus oil, grapefruit oil, lime oil, olibanum oil, orange oil, patchouli essence, rose absolue, rosemary oil, thyme oil and tuberose absolue;
   and
   at least one substance of group (D) in a total amount of between 0.01 and 35 wt.-%, preferably of between 0.2 and 20 wt.-%, especially preferably of between 0.5 and 10 wt.-%,
   wherein group (D) consists of compounds with a fruity odor impression, selected from the group consisting of (2E)-3,7-Dimethylocta-2,6-dienal, 5-hexyltetrahydrofuran-2-one, Methoxyphenylbutanone, 6,6-Dimethoxy-2,5,5-trimethylhex-2-ene, hexyl acetate, decanal, ethyl butanoate, 3-methylbutyl acetate, 2,4-dimethyl-4,4a,5,9b-tetrahydoindeno[1,2-d][1,3]dioxine, 2-methyl-4-propyl-1,3-oxathiane, allyl heptanoate and mixtures thereof;
   and
   optionally at least one substance of group (E) in a total amount of between 0.01 and 35 wt.-%, preferably of between 0.2 and 20 wt.-%, especially preferably of between 0.5 and 10 wt.-%,
   wherein group (E) consists of compounds having green and/or herbal odor impression selected from the group consisting of 7-methyl-1,5-benzodioxepin-3-one, 1-methyl-4-(4-methylpent-3-enyl)cyclohex-3-ene-1-carbaldehyde, 2,4-Dimethylcyclohex-3-ene-1-carbaldehyde, undec-10-enal, 5-isopropenyl-2-methyl-cyclohex-2-en-1-one, 2,4,4,7-tetramethyloct-6-en-3-one, 4-allyl-2-methoxy-phenol, 2,6-dimethylhept-5-enal, (E)-4-methyldec-3-en-5-ol and mixtures thereof;
ii) contacting the fragrance composition with the subject, preferably a human, in an amount sufficient to enhance the mood of the subject/human.

One preferred embodiment of the present invention relates to the method according to the invention for imparting a positive mood, preferably a joyful mood, in a subject, preferably a human, wherein the fragrance composition comprises or consists of:
i) Providing a fragrance composition, wherein the fragrance composition comprises or consists of:
   at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (A) consists of compounds with a bloomy odor impression selected from the group consisting of 7-hydroxy-3,7-dimethyl-octanal, (6E)-3,7-dimethylnona-1,6-dien-3-ol, beta,beta-3-Trimethyl-benzenepropanol, methyl-2-aminobenzoate, (E)-4-(2,6,6-trimethylcyclohex-2-en-1-yl)but-3-en-2-one, benzyl acetate, 3-(4-ethylphenyl)-2,2-dimethylpropanal, (2E)-3,7-dimethylocta-2,6-dien-1-ol, 3-(1,3-benzodioxol-5-yl)-2-methyl-propanal, [(Z)-hex-3-enyl]-2-hydroxybenzoate, (E)-4-(2,6,6-trimethylcyclohexen-1-yl)but-3-en-2-one, (+)-3-(4-Isopropylcyclohexen-1-yl)propanal, methyl 2-[(7-hydroxy-3,7-dimethyloctylidene)amino]benzoate, ethyl 2,6,6-trimethylcyclohexa-1,3-diene-1-carboxylate, 3-(3-isopropylphenyl)butanal, 2-(phenylmethylidene)octanal, (E)-1-(2,4,4-trimethylcyclohexen-1-yl)but-2-en-1-one, 3,7-dimethylocta-1,6-dien-3-ol, 2-methyl-4-phenyl-butan-2-ol, 2-methyl-5-phenylpentanol, 2,2,5-trimethyl-5-pentyl-cyclopentanone, 3-(4-methoxyphenyl)-2-methyl-propanal and mixtures thereof,
   and
   at least one substance of group (B) in a total amount of between 0.01 and 50 wt.-%, preferably of between 0.2 and 35 wt.-%, especially preferably of between 0.5 and 20 wt.-%,
   wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, Oxacyclohexadecen-2-one (mixture of isomers), Pentadecan-15-olide, (E)-3-methyl-4-(2,6,6-trimethylcyclohex-2-en-1-yl)but-3-en-2-one, 1-[2,3,8,8-tetramethyl-1,3,4,5,6,7-hexanhydronaphthalen-2-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, (E)-2-methyl-3-phenyl-prop-2-en-1-ol, 4-(1-ethoxyvinyl)-3,3,5,5-tetramethyl-cyclohexanone, (E)-3,4,5,6,6-pentamethylhept-3-en-2-one, 1-(1,1,2,4,4,7-hexamethyltetralin-6-yl)ethanone and mixtures thereof;
   and
   at least one substance of group (C) in a total amount of between 0.01 and 35 wt.-%, preferably of between 0.2 and 20 wt.-%, especially preferably of between 0.5 and 10 wt.-%,
   wherein group (C) consists of compounds having an earthy and/or sweet odor impression selected from the group consisting of chromen-2-one, 2-ethyl-3-hydroxy-pyran-4-one, 3-ethoxy-4-hydroxy-benzaldehyde, 2-Methyl-5-propyl-cyclohex-2-en-1-one, 3,6-Dimethyl-3H-benzofuran-2-one, 1,3-benzodioxole-5-carbaldehyde, 5-pentyltetrahydrofuran-2-one, 1-methoxy-4-[(e)-prop-1-enyl]benzene, 4-methoxybenzaldehyde, 4-hydroxy-2,5-dimethylfuran-3-one, 3-hydroxy-2-methyl-4h-pyran-4-one, 4-hydroxy-3-methoxy-benzaldehyde, (4-formyl-2-methoxy-phenyl) 2-methylpropanoate and mixtures thereof;
   and
   at least one essential oil and/or essence and/or absolue selected from the group consisting of bergamot oil, lavender oil, vetiver oil, Artemisia oil, artichoke essence, elemi oil, ginger oil, lemon oil, lemongrass oil, mandarin oil, black pepper oil, red berry extract, clove essence, vanilla absolue, ylang ylang oil, benzaldehyde, cardamom oil, cypress oil, eucalyptus oil, geranium oil, grapefruit oil, jasmin absolue, lime oil, olibanum oil, range flower absolue, orange oil, rose absolue, rosemary oil, sage oil, sandalwood oil, thyme oil and tonka bean absolue.
   and
   optionally at least one substance of group (D) in a total amount of between 0.01 and 35 wt.-%, preferably of between 0.2 and 20 wt.-%, especially preferably of between 0.5 and 10 wt.-%,
   wherein group (D) consists of compounds with a fruity odor impression, selected from the group consisting of allyl 3-cyclohexylpropanoate, (2E)-3,7-Dimethylocta-2,6-dienal, 5-hexyltetrahydrofuran-2-one, Methoxyphenylbutanone, 5-heptyltetrahydrofuran-2-one, 6,6-Dimethoxy-2,5,5-trimethylhex-2-ene, hexyl acetate, isopropyl tetradeconate, 2-tert-Butylcyclohexyl acetate, 5-Hexyldihydro-4-methylfuran-2(3H)-one, ethyl-2-methylpentanoate, mixture of diethyl (E/Z)-2-methylbut-2-enedioate and diethyl 2-methylenebutanedioate, ethyl rac-(2R,3R)-3-methyl-3-phenyl-oxirane-2-carboxylate, allyl hexanoate, 3-methylbutyl acetate, 2,4-dimethyl-4,4a,5,9b-tetrahydoindeno[1,2-d][1,3]dioxine, allyl heptanoate and mixtures thereof;
   and
   optionally at least one substance of group (E) in a total amount of between 0.01 and 35 wt.-%, preferably of between 0.2 and 20 wt.-%, especially preferably of between 0.5 and 10 wt.-%,
   wherein group (E) consists of compounds having green and/or herbal odor impression selected from the group consisting 7-methyl-1,5-benzodioxepin-3-one, Hex-5-enyl 2-m,ethylbutanoate, [(Z)-hex-3-enyl]acetate, (Z)-Hex-3-en-1-yl methyl carbonate, 5-isopropenyl-2-methyl-cyclohex-2-en-1-one, 2,4,4,7-tetramethyloct-6-en-3-one, 3a,4,5,6,7,7a-hexahydro-4,7-methanoinden-6-yl acetate, (E)-4-methyldec-3-en-5-ol of and mixtures thereof;
ii) contacting the fragrance composition with the subject, preferably a human, in an amount sufficient to enhance the mood of the subject/human.

Yet another preferred embodiment relates to the method according to the invention for imparting a positive mood, preferably a relaxing mood, in a subject, preferably a human, consisting of or comprising the steps of:
i) Providing a fragrance composition, wherein the fragrance composition comprises or consists of:
   at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (A) consists of compounds with a bloomy odor impression selected from the group consisting of 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, beta,beta-3-Trimethyl-benzenepropanol, methyl-2-aminobenzoate, (E)-4-(2,6,6-trimethylcyclohex-2-en-1-yl)but-3-en-2-one, benzyl 2-hydroxybenzoate, methyl-2-[RAC-(1S,2S)-3-oxo-2-pentyl-cyclopentyl]acetate, (+)-3-(4-Isopropylcyclohexen-1-yl)propanal, (1,5-dimethyl-1-vinyl-hex-4-enyl)acetate, 2-phenylethanol, 4-Isopentylcyclohexanol, methyl 2-[(7-hydroxy-3,7-dimethyloctylidene)amino]benzoate, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 2,3-dihydro-1,1-dimethyl-1h-indene-ar-propanal, (2e)-2-methyl-6-methylene-octa-2,7-dien-1-ol, 5-hexyl-5-methyl-tetrahydrofuran-2-one, 4-(propan-2-yl)cyclohexyl]methanol, 3-methyl-5-phenylpentan-1-ol, 2-methyl-5-phenylpentanol, 3-(4-methoxyphenyl)-2-methyl-propanal and mixtures thereof,
   and optionally at least one substance of group (B) in a total amount of between 0.01 and 50 wt.-%, preferably of between 0.2 and 35 wt.-%, especially preferably of between 0.5 and 20 wt.-%,
   wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, 1,4-dioxacycloheptadecane-5,17-dione, 4,6,6,7,8,8-hexamethyl-1,3,4,7-tetrahydrocyclopenta[G]isochromene, Oxacyclohexadecen-2-one (mixture of isomers), Pentadecan-15-olide, 8-methoxy-2,6,6,8-tetramethyl-tricyclo[5.3.1.01,5]undecane, [2-[1-(3,3-dimethylcyclohexyl)ethoxy]-2-methyl-propyl]propanoate, (E)-3-methyl-4-(2,6,6-trimethylcyclohex-2-en-1-yl)but-3-en-2-one, (5Z)-3-methylcyclopentadec-5-en-1-one, (2E)-2-Ethyl-4-(2,2,3)-trimethylcyclopent-3-en-1-yl) but-2-en-1-ol, 1-[(2E,5E,9E)-2,6,10-trimethylcyclododeca-2,5,9-trien-1-yl]ethanone, oxacycloheptadec-10-en-2-one, 2-ethoxy-2,6,6-trimethyl-9-methylene-bicyclo[3.3.1]nonane, (E)-2-methyl-3-phenyl-prop-2-en-1-ol, rac-(E,2R,3R)-3-methyl-5-(2,2,3-trimethylcyclopent-3-en-1-yl)pent-4-en-2-ol, 4-(1,7,7-trimethyl-2-bucyclo[3.1.1]heptanyl)ccyclohexan-1-ol, 4-(1-ethoxyvinyl)-3,3,5,5-tetramethyl-cyclohexanone, 1-(2,6,6,8-tetramethyl-9-tricyclo[5.3.1.01,5]undec-8-enyl)ethanone, 1-(1,1,2,4,4,7-hexamethyltetralin-6-yl)ethanone, oxacycloheptadec-10-en-2-one, DL Ambre 83 SMP N, methyl-2,4-dihydroxy-3,6-dimethyl-benzoate, (4-tert-butylcyclohexyl) acetate and mixtures thereof,
   and
   at least one substance of group (C) in a total amount of between 0.01 and 99 wt.-%, preferably of between 0.01 and 80 wt.-%, preferably of between 0.2 and 50 wt.-%, especially preferably of between 0.5 and 10 wt.-%,
   wherein group (C) consists of compounds having an earthy and/or sweet odor impression selected from the group consisting of chromen-2-one, 4-methoxybenzaldehyde, 3-hydroxy-2-methyl-4H-pyran-4-one, 4,6,6,7,8,8-hexamethyl-1,3,4,7-tetrahydrocyclopenta[G]isochromene, 4-hydroxy-3-methoxy-benzaldehyde, (4-formyl-2-methoxy-phenyl) 2-methylpropanoate, [2-[1-(3,3-dimethylcyclohexyl)ethoxy]-2-methyl-propyl]propanoate, (E)-4-(2,6,6-trimethylcyclohex-2-en-1-yl)but-3-en-2-one, pentadecan-15-olide and mixtures thereof;
   and
   at least one essential oil selected from the group consisting of at least one essential oil and/or essence and/or absolue selected from the group consisting of lavender oil, cedarwood oil, cypress oil, eucalyptus oil, geranium oil, lime oil, orange flower absolue, rose absolue, rosemary oil, sage oil and tonka bean absolue;
   and
   optionally at least one substance of group (D) in a total amount of between 0.01 and 35 wt.-%, preferably of between 0.2 and 20 wt.-%, especially preferably of between 0.5 and 10 wt.-%,
   wherein group (D) consists of compounds with a fruity odor impression, selected from the group consisting of 5-heptyltetrahydrofuran-2-one, 5-hexyldihydro-4-methylfuran-2(3h)-one, ethyl 3-oxobutanoate, 5-octyltetrahydrofuran-2-one, ethyl 2-(2-methyl-1,3-dioxolan-2-yl)acetate, gamma-dodecalactone and mixtures thereof;
   and
   optionally at least one substance of group (E) in a total amount of between 0.01 and 35 wt.-%, preferably of between 0.2 and 20 wt.-%, especially preferably of between 0.5 and 10 wt.-%,
   wherein group (E) consists of compounds having green and/or herbal odor impression selected from the group consisting of 4,11,11-trimethyl-8-methylene-bicyclo[7.2.0]undec-4-ene, methyl-2,4-dihydroxy-3,6-dimethyl-benzoate, 5-isopropenyl-2-methyl-cyclohex-2-en-1-one, 3a,4,5,6,7,7a-hexahydro-4,7-methanoinden-6-yl acetate, 2-ethoxy-4-(methoxymethyl)phenol, (4-tert-butylcyclohexyl) acetate, (E)-4-methyldec-3-en-5-ol and mixtures thereof;
ii) contacting the fragrance composition with the subject, preferably a human, in an amount sufficient to enhance the mood of the subject/human.

One preferred embodiment of the present invention relates to a method according to the present invention for imparting a positive mood, preferably a self-confident mood, in a subject, preferably a human, consisting of or comprising the steps of:
i) Providing a fragrance composition, wherein the fragrance composition comprises or consists of:
   at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (A) consists of compounds with a bloomy odor impression selected from the group consisting of 3-(4-tert-butylphenyl)propanal, 7-hydroxy-3,7-dimethyl-octanal, 3,7-dimethyloct-6-en-1-ol, 3-(4-isopropylphenyl)-2-methyl-propanal, beta,beta-3-Trimethyl-benzenepropanol, (E)-4-(2,6,6-trimethylcyclohex-2-en-1-yl)but-3-en-2-one, benzyl 2-hydroxybenzoate, (+)-3-(4-Isopropylcyclohexen-1-yl)propanal, 2-phenylethanol, ethyl 2,6,6-trimethylcyclohexa-1,3-diene-1-carboxylate, 5-hexyl-5-methyl-tetrahydrofuran-2-one and mixtures thereof,
   and
   at least one substance of group (B) in a total amount of between 0.01 and 50 wt.-%, preferably of between 0.2 and 35 wt.-%, especially preferably of between 0.5 and 20 wt.-%,
   wherein group (B) consists of compounds with a woody and/or balsam odor impression, selected from the group consisting of 1,1,2,3,3,-pentamethyl-2,5,6,7-tetrahydroinden-4-one, 1,4-dioxacycloheptadecane-5,17-dione, Oxacyclohexadecen-2-one (mixture of isomers), Pentadecan-15-olide, [2-[1-(3,3-dimethylcyclohexyl)ethoxy]-2-methyl-propyl]propanoate, Oxacycloheptadec-10-en-2-one, rac-(E,2R,3R)-3-methyl-5-(2,2,3-trimethylcyclopent-3-en-1-yl)pent-4-en-2-ol and mixtures thereof;
   and
   at least one substance of group (C) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (C) consists of compounds having an earthy and/or sweet odor impression selected from the group consisting of chromen-2-one, 4-methoxybenzaldehyde and mixtures thereof, and mixtures thereof;
   and
   optionally at least one essential oil and/or essence and/or absolue selected from the group consisting of lavender oil, cedarwood oil, geranium oil, jasmin absolue and sage oil;
   and
   optionally at least one substance of group (D) in a total amount of between 0.01 and 35 wt.-%, preferably of between 0.2 and 20 wt.-%, especially preferably of between 0.5 and 10 wt.-%,
   wherein group (D) consists of compounds with a fruity odor impression, selected from the group consisting of 5-heptyltetrahydrofuran-2-one, 5-Hexyldihydro-4-methylfuran-2(3H)-one, ethyl 2-(2-methyl-1,3-dioxolan-2-yl)acetate and mixtures thereof;
   and
   optionally at least one substance of group (E) in a total amount of between 0.01 and 35 wt.-%, preferably of between 0.2 and 20 wt.-%, especially preferably of between 0.5 and 10 wt.-%,
   wherein group (E) consists of compounds having green and/or herbal odor impression selected from the group consisting of 4,11,11-trimethyl-8-methylene-bicyclo[7.2.0]undec-4-ene, 2-ethoxy-4-(methoxymethyl)phenol and mixtures thereof;
ii) contacting the fragrance composition with the subject, preferably a human, in an amount sufficient to enhance the mood of the subject/human.

Another preferred embodiment relates to the method according to the invention for imparting a well-being mood, in a subject, preferably a human, consisting of or comprising the steps of:
i) Providing a fragrance composition, wherein the fragrance composition comprises or consists of:
   at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
   wherein group (A) consists of compounds with a bloomy odor impression selected from the group consisting of 7-hydroxy-3,7-dimethyl-octanal, beta,beta-3-Trimethyl-benzenepropanol, (E)-4-(2,6,6-trimethylcyclohex-2-en-1-yl)but-3-en-2-one, benzyl acetate, 3-(1,3-benzodioxol-5-yl)-2-methyl-propanal, (E)-4-(2,6,6-trimethylcyclohexen-1-yl)but-3-en-2-one, (+)-3-(4-Isopropylcyclohexen-1-yl)propanal, methyl 2-[(7-hydroxy-3,7-dimethyloctylidene)amino]benzoate, ethyl 2,6,6-trimethylcyclohexa-1,3-diene-1-carboxylate, 3-(3-isopropylphenyl)butanal, 2-(phenylmethylidene)octanal, 2-methyl-4-phenyl-butan-2-ol, 2-methyl-5-phenylpentanol, 3-(4-methoxyphenyl)-2-methyl-propanal and mixtures thereof,
   and
   at least one substance of group (B) in a total amount of between 0.01 and 50 wt.-%, preferably of between 0.2 and 35 wt.-%, especially preferably of between 0.5 and 20 wt.-%,
   wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, Oxacyclohexadecen-2-one (mixture of isomers), Pentadecan-15-olide, (E)-3-methyl-4-(2,6,6-trimethylcyclohex-2-en-1-yl)but-3-en-2-one, 1-[2,3,8,8-tetramethyl-1,3,4,5,6,7-hexanhydronaphthalen-2-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, (E)-2-methyl-3-phenyl-prop-2-en-1-ol, 4-(1-ethoxyvinyl)-3,3,5,5-tetramethyl-cyclohexanone, (e)-3,4,5,6,6-pentamethylhept-3-en-2-one, 1-(1,1,2,4,4,7-hexamethyltetralin-6-yl)ethanone and mixtures thereof;
   and
   at least one substance of group (C) in a total amount of between 0.01 and 35 wt.-%, preferably of between 0.2 and 20 wt.-%, especially preferably of between 0.5 and 10 wt.-%,
   wherein group (C) consists of compounds having an earthy and/or sweet odor impression selected from the group consisting of chromen-2-one, 2-ethyl-3-hydroxy-pyran-4-one, 3-ethoxy-4-hydroxy-benzaldehyde, 2-Methyl-5-propyl-cyclohex-2-en-1-one, 3,6-Dimethyl-3H-benzofuran-2-one, 1,3-benzodioxole-5-carbaldehyde, 5-pentyltetrahydrofuran-2-one, 1-methoxy-4-[(E)-prop-1-enyl]benzene, 4-methoxybenzaldehyde, 4-hydroxy-2,5-dimethylfuran-3-one, 3-hydroxy-2-methyl-4h-pyran-4-one, 4-hydroxy-3-methoxy-benzaldehyde, (4-formyl-2-methoxy-phenyl) 2-methylpropanoate and mixtures thereof;
   and
   at least one essential oil and/or essence and/or absolue selected from the group consisting of bergamot oil, lavender oil, vetiver oil, artichoke essence, elemi oil, ginger oil, lemongrass oil, mandarin oil, black pepper oil, red berry extract, vanilla absolue, ylang ylang oil, cardamom oil, eucalyptus oil, geranium oil, grapefruit oil, jasmin absolue, lime oil, olibanum oil, orange flower absolue, orange oil, rose absolue, rosemary oil, sage oil, sandalwood oil, thyme oil and tonka bean absolue;
   and
   optionally at least one substance of group (D) in a total amount of between 0.01 and 35 wt.-%, preferably of between 0.2 and 20 wt.-%, especially preferably of between 0.5 and 10 wt.-%,
   wherein group (D) consists of compounds with a fruity odor impression, selected from the group consisting of allyl 3-cyclohexylpropanoate, (2E)-3,7-Dimethylocta-2,6-dienal, 5-hexyltetrahydrofuran-2-one, Methoxyphenylbutanone, 5-heptyltetrahydrofuran-2-one, 6,6-Dimethoxy-2,5,5-trimethylhex-2-ene, hexyl acetate, isopropyl tetradeconate, ethyl-2-methylpentanoate, ethyl rac-(2R,3R)-3-methyl-3-phenyl-oxirane-2-carboxylate, allyl hexanoate, 3-methylbutyl acetate, allyl heptanoate and mixtures thereof;
   and
   optionally at least one substance of group (E) in a total amount of between 0.01 and 35 wt.-%, preferably of between 0.2 and 20 wt.-%, especially preferably of between 0.5 and 10 wt.-%,
   wherein group (E) consists of compounds having green and/or herbal odor impression selected from the group consisting of 7-methyl-1,5-benzodioxepin-3-one, hex-5-enyl 2-methylbutanoate, (Z)-Hex-3-en-1-yl methyl carbonate, 5-isopropenyl-2-methyl-cyclohex-2-en-1-one, 3a,4,5,6,7,7a-hexahydro-4,7-methanoinden-6-yl acetate, (E)-4-methyldec-3-en-5-ol and mixtures thereof;
ii) contacting the fragrance composition with the subject, preferably a human, in an amount sufficient to enhance the mood of the subject/human.

Another preferred embodiment relates to a method according to the invention, wherein the fragrance composition provided in step i) is contained in a perfumed product.

Another embodiment relates to a method according to the invention, wherein the fragrance composition provided in step i) is contained in a perfumed product in a total amount in the range of from 0.01 to 35 wt.-%, preferably of from 0.2 to 25 wt.-%, more preferably of from 0.3 to 20 wt.-%, based on the total weight of the perfumed product.

Yet another embodiment relates to a method according to the invention, wherein the fragrance composition provided in step i) is contained in a perfumed product and the perfumed product is selected from the group consisting of fine fragrance, personal cosmetic, toiletry, and healthcare products such as dry and wet wipes, washes, baths, shampoos, gels, soaps, mousses, cleansing compositions, bath oils, other bath compositions that may be added to a bath, other wash compositions that may be used directly on the skin, leave-on personal care products include personal cosmetic, toiletry, and healthcare products such as sticks, balms, mousses, sprays, lotions, creams, gels, powders, oils, waxes, perfumes, other personal care compositions that may be applied to the skin and not rinsed off, or home or laundry care products such as fabric detergents, fabric softeners, linen sprays, air care products, diffusers, devices or other products that may be used in the home for the purpose of cleaning surfaces, fabrics or perfuming the air.

Another aspect of the present invention relates to a fragrance composition for conveying a positive mood, preferably an attractive, protective, joyful, fresh, energizing, relaxing, sensual, self-confident and/or well-being mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 3-(4-tert-butylphenyl)propanal, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-Trimethyl-benzenepropanol, methyl-2-aminobenzoate, (E)-4-(2,6,6-trimethylcyclohex-2-en-1-yl)but-3-en-2-one, 3-(4-ethylphenyl)-2,2-dimethylpropanal, 3-(1,3-benzodioxol-5-yl)-2-methyl-propanal, (2E)-3,7-dimethylocta-2,6-dien-1-ol, methyl-2-[RAC-(1S,2S)-3-oxo-2-pentyl-cyclopentyl]acetate, 1-phenylethyl acetate, benzyl acetate, 1,3-benzodioxole-5-carbaldehyde, [(Z)-hex-3-enyl]-2-hydroxybenzoate, (methyl-2-[RAC-(1S,2S)-3-oxo-2-pentyl-cyclopentyl]acetate, (1,5-dimethyl-1-vinyl-hex-4-enyl)acetate, 2-phenylethanol, 4-Isopentylcyclohexanol, benzyl 2-hydroxybenzoate, 4-cyclohexylbutan-2-ol, (E)-1-(2,6,6-trimethylcyclohexan-1,3-dien-1-yl)but-2-en-1-one, (2E)-3,7-dimethylocta-2,6-dienyl]acetate, (E)-4-(2,6,6-trimethylcyclohexen-1-yl)but-3-en-2-one, (+)-3-(4-Isopropylcyclohexen-1-yl)propanal, 2,6-dimethyloct-7-en-2-ol 7-methyl-1,5-benzodioxepin-3-one, 1-methyl-4-(4-methylpent-3-enyl)cyclohex-3-ene-1-carbaldehyde, (E)-1-(2,6,6-trimethylcyclohex-2-en-1-yl)but-2-en-1-one, 2-isobutyl-4-methyl-tetrahydropyran-4-ol, [(2E)-3,7-dimethylocta-2,6-dienyl]acetate, indole, (1E)-1-(2,6,6-trimethylcyclohex-2-en-1-yl)hepta-1,6-dien-3-one, methyl 2-[(7-hydroxy-3,7-dimehyloctylidene)amino]benzoate, rac-(e)-1-[rac-(1r,2s)-2,6,6-trimethylcyclohex-3-en-1-yl]but-2-en-1-one, 2-methyl-1-phenyl-propan-2-ol, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 2,3-dihydro-1,1-dimethyl-1h-indene-ar-propanal, (E)-1-(2,4,4-trimethylcyclohexen-1-yl)but-2-en-1-one, (2E)-2-methyl-6-methylene-octa-2,7-dien-1-ol, 5-hexyl-5-methyl-tetrahydrofuran-2-one, 3,7-dimethylocta-1,6-dien-3-ol, 4-(propan-2-yl)cyclohexyl]methanol, 1-naphthalen-2-ylethanone, 2-ethoxynaphtalene, 2-methoxynaphtalene, 3-methyl-2-benzofuran-1-yl)ethanone, 2-phenoxyethyl 2-methylpropanoate, 3-methyl-5-phenylpentan-1-ol, 2-methyl-4-phenyl-butan-2-ol, (2,2,2-trichloro-1-phenyl-ethyl) acetate, 2-methyl-5-phenylpentanol, rac-(2r,4s)-4-methyl-2-(2-methylprop-1-enyl)tetrahydropyran, 2,2,5-trimethyl-5-pentyl-cyclopentanone, 3-(4-methoxyphenyl)-2-methyl-propanal, (6E)-3,7,11-trimethyldodeca-1,6,10-trien-3-ol, (6Z)-3,7,11-trimethyldodeca-1,6,10-trien-3-ol and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-tetramethyldodecahydronaphtho[2,1-b]furan, 1,1,2,3,3,-pentamethyl-2,5,6,7-tetrahydroinden-4-one, 8-methoxy-2,6,6,8-tetramethyl-tricyclo[5.3.1.01,5]undecane, (E)-3-methyl-4-(2,6,6-trimethylcyclohex-2-en-1-yl)but-3-en-2-one, (2E)-2-Ethyl-4-(2,2,3)-trimethylcyclopent-3-en-1-yl)but-2-en-1-ol, 1-[(2E,5E,9E)-2,6,10-trimethylcyclododeca-2,5,9-trien-1-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, 1-[2,3,8,8-tetramethyl-1,3,4,5,6,7-hexanhydronaphthalen-2-yl]ethanone, (Ethoxymethoxy)cyclododecane, dioxol-4H-4A,9-methanoazuleno(5,6-D)-octahydro-2-propyl-2,5,8,8,9A-pentamethyl-1,3, (E/Z)-4-Ethyloct-2/3-enenitrile, 3,4-dimethylbenzonitrile, (2E)-3-phenylprop-2-en-1-ol, 2,3,6-trimethylcyclohexanol, oxacycloheptadec-10-en-2-one, (4aR,5R,7aS,9R)-octahydro-2,2,5,8,8,9a-hexamethyl-4H-4a,9-methanoazuleno(5,6-d)-1,3-dioxole, 2-ethoxy-2,6,6-trimethyl-9-methylene-bicyclo[3.3.1]nonane, (E)-2-methyl-3-phenyl-prop-2-en-1-ol, rac-(E,2R,3R)-3-methyl-5-(2,2,3-trimethylcyclopent-3-en-1-yl)pent-4-en-2-ol, 4-(1,7,7-trimethyl-2-bucyclo[3.1.1]heptanyl)ccyclohexan-1-ol, 6-butan-2-ylquinoline, 4-(1-ethoxyvinyl)-3,3,5,5-tetramethyl-cyclohexanone, (E)-3,4,5,6,6-pentamethylhept-3-en-2-one, 2-(4-methylcyclohex-3-en-1-yl)propan-2-ol, 1-(2,6,6,8-tetramethyl-9-tricyclo[5.3.1.01,5]undec-8-enyl)ethanone, rac-(3′R,6′R,9′R)-2′,2′,8′,8′-tetramethylspiro[1,3-dioxolane-2,10′-octahydro-1 h-2,4a-methanonapthalene], 1-(1,1,2,4,4,7-hexamethyltetralin-6-yl)ethanone and mixtures thereof,
and
at least one substance of group (C) in a total amount of between 0.01 and 35 wt.-%, preferably of between 0.2 and 20 wt.-%, especially preferably of between 0.5 and 10 wt.-%,
wherein group (C) consists of compounds having an earthy and/or sweet odor impression selected from the group consisting of chromen-2-one, 2-ethyl-3-hydroxy-pyran-4-one, (5Z)-3-methylcyclopentadec-5-en-1-one, 3-ethoxy-4-hydroxy-benzaldehyde, 1,4-dioxacycloheptadecane-5,17-dione, 4,6,6,7,8,8-hexamethyl-1,3,4,7-tetrahydrocyclopenta[G]isochromene, oxacyclohexadecen-2-one (mixture of isomers), Pentadecan-15-olide, [2-[1-(3,3-dimethylcyclohexyl)ethoxy]-2-methyl-propyl]propanoate, 3,6-Dimethyl-3H-benzofuran-2-one, (5Z)-3-methylcyclopentadec-5-en-1-one, 4,6,6,7,8,8-hexamethyl-1,3,4,7-tetrahydrocyclopenta[G]isochromene, 2-methyl-5-propyl-cyclohex-2-en-1-one, 1,3-benzodioxole-5-carbaldehyde, 1-pyrazin-2-ylethanone, 5-pentyltetrahydrofuran-2-one, 1-methoxy-4-[(e)-prop-1-enyl]benzene, 4-methoxybenzaldehyde, 4-hydroxy-2,5-dimethylfuran-3-one, 3-hydroxy-2-methyl-4h-pyran-4-one, 2-(4-methylthiazol-5-yl)ethanol, 4-hydroxy-3-methoxy-benzaldehyde, (4-formyl-2-methoxy-phenyl)-2-methylpropanoate, and mixtures thereof, and
at least one essential oil and/or essence and/or absolue selected from the group consisting of bergamot oil, ginger root extract, vetiver oil, lemon oil, artemisia oil, cinnamon bark oil, elemi oil, ginger oil, lemongrass oil, longoza oil, mandarin oil, pepper black oil, vanilla absolue, red berry extract, bay oil, cedarwood oil hemp oil, pink pepper leaf oil, amyris oil, cardamom oil, carrot seed oil, cypress oil, davana oil, geranium oil, grapefruit oil, lime oil, olibanum oil, palmarosa oil, sage oil, sandalwood oil, thyme oil, artichoke essence, asparagus essence, cauliflower essence, hemp essence, leek essence, clove essence, passionfruit essence, amber base, red apple essence, benzaldehyde, Styrax tonkinensis essence, camphor oil, eucalyptus oil, jasmin oil, jasmin absolue, orange oil, rosemary oil, galbanum essence, orange flower absolue, patchouli essence, rose absolue, tonka bean absolue, tuberose absolue, rose bourboniana absolute

Another preferable embodiment relates to a fragrance composition according to the invention for conveying a positive mood, preferably a protective mood, in a subject, preferably a human, wherein the fragrance composition comprises or consists of:

Optionally at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from at least one substance of group (A) in a total amount of between 0.01 and 35 wt.-%, preferably of between 0.2 and 20 wt.-%, especially preferably of between 0.5 and 10 wt.-%, 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-trimethyl-benzenepropanol, methyl-2-aminobenzoate, (E)-4-(2,6,6-trimethylcyclohex-2-en-1-yl)but-3-en-2-one, methyl 2-[(7-hydroxy-3,7-dimethyloctylidene)amino]benzoate, 2,6-dimethylheptan-2-ol, 5-hexyl-5-methyl-tetrahydrofuran-2-one, 3,7-dimethylocta-1,6-dien-3-ol, 4-(propan-2-yl)cyclohexyl]methanol, 3-(4-methoxyphenyl)-2-methyl-propanal and mixtures thereof, preferably selected from 2,6-dimethylheptan-2-ol,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 1,1,2,3,3,-pentamethyl-2,5,6,7-tetrahydroinden-4-one, 4,6,6,7,8,8-hexamethyl-1,3,4,7-tetrahydrocyclopenta[g]isochromene, oxacyclohexadecen-2-one (mixture of isomers), pentadecan-15-olide, [2-[1-(3,3-dimethylcyclohexyl)ethoxy]-2-methyl-propyl]propanoate, 1-[(2E,5E,9E)-2,6,10-trimethylcyclododeca-2,5,9-trien-1-yl]ethanone, 2,2,6,8-tetramethyltricyclo[5.3.1.03,8]undecan-3-ol, 1-(2,6,6,8-tetramethyl-9-tricyclo[5.3.1.01,5]undec-8-enyl)ethanone and mixtures thereof,
and
at least one substance of group (C) in a total amount of between 0.01 and 99 wt.-%, preferably of between 0.01 and 35 wt.-%, preferably of between 0.2 and 20 wt.-%, especially preferably of between 0.5 and 10 wt.-%,
wherein group (C) consists of compounds having an earthy and/or sweet odor impression selected from the group consisting of 3-ethoxy-4-hydroxy-benzaldehyde, (4-formyl-2-methoxy-phenyl) 2-methylpropanoate and mixtures thereof;
and
at least one essential oil and/or essence and/or absolue selected from the group consisting of lavender oil, eucalyptus oil, geranium oil, rose absolue, sage oil and tonka bean absolue.

One preferred embodiment of the present invention relates to a fragrance composition for conveying a positive mood, preferably a sensual mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 2,6-dimethyloct-7-en-2-ol, beta, beta-3-Trimethyl-benzenepropanol and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, 1,4-dioxacycloheptadecane-5,17-dione, 8-methoxy-2,6,6,8-tetramethyl-tricyclo[5.3.1.01,5]undecane, 1-[2,3,8,8-tetramethyl-1,3,4,5,6,7-hexanhydronaphthalen-2-yl]ethanone, 2,2,6,8-tetramethyltricyclo[5.3.1.03,8]undecan-3-ol, 2-ETHOXY-2,6,6-trimethyl-9-methylene-bicyclo[3.3.1]nonane, 4-(1-ethoxyvinyl)-3,3,5,5-tetramethyl-cyclohexanone, 1-(2,6,6,8-tetramethyl-9-tricyclo[5.3.1.01,5]undec-8-enyl)ethanone, 1-(1,1,2,4,4,7-hexamethyltetralin-6-yl)ethanone and mixtures thereof;
and
at least one substance of group (C) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (C) consists of compounds having an earthy and/or sweet odor impression selected from the group consisting of 3,6-dimethyl-3H-benzofuran-2-one, 1,4-dioxacycloheptadecane-5,17-dione, 4-hydroxy-3-methoxy-benzaldehyde, (4-formyl-2-methoxy-phenyl) 2-methylpropanoate and mixtures thereof,
and
at least one essential oil and/or essence and/or absolue selected from the group consisting of vetiver oil, vanilla absolue, jasmin absolue, patchouli oil, rose absolue and sandalwood oil.

Another preferred embodiment relates a fragrance composition according to the invention,
wherein the fragrance composition additionally comprises at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of allyl 3-cyclohexylpropanoate, 5-hexyltetrahydrofuran-2-one, methoxyphenylbutanone, (2E)-3,7-Dimethylocta-2,6-dienal, 5-heptyltetrahydrofuran-2-one, hexyl acetate, isopropyl tetradeconate, 6,6-dimethoxy-2,5,5-trimethylhex-2-ene, ethyl-3-oxobutanoate, ethyl-2-methylpentanoate, 5-hexyldihydro-4-methylfuran-2(3H)-one, 2-tert-butylcyclohexyl acetate, diethyl 2-methylenebutanedioate, 2-bicyclo[2.2.1]hept-5-enyl acetate, ethyl 3-oxobutanoate, linolal, mixture of diethyl (E/Z)-2-methylbut-2-enedioate and diethyl 2-methylenebutanedioate, mixture of (5-isopropenyl-2-methyl-cyclohex-2-en-1-yl) acetate and (5-isopropenyl-2-methyl-cyclohexyl) acetate, ethyl tetrahydrofuran-2-carboxylate, decanal, ethyl rac-(2r,3r)-3-methyl-3-phenyl-oxirane-2-carboxylate, allyl hexanoate, (1,1-dimethyl-2-phenyl-ethyl) acetate, 5-octyltetrahydrofuran-2-one, ethyl butanoate, ethyl 2-methylbutanoate, 3-methylbutyl acetate, ethyl 2-(2-methyl-1,3-dioxolan-2-yl)acetate, 2,4-dimethyl-4,4a,5,9b-tetrahydoindeno[1,2-d][1,3]dioxine, 2-methyl-4-propyl-1,3-oxathiane, 2,4,6-trimethyl-4-phenyl-1,3-dioxane, allyl heptanoate and mixtures thereof.

Yet another preferred embodiment relates to a fragrance composition according to the invention, wherein the fragrance composition additionally comprises
at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of, allyl-2isopentyloxyacetate, allyl (cyclohexyloxy)acetate, 1-(5,5-dimethylcyclohexen-1-yl)pent-4-en-1-one, 2,4-dimethylcyclohex-3-ene-1-carbaldehyde, (Z)-hex-3-en-1-yl methyl carbonate, hex-5-enyl 2-methylbutanoate, (Z)-hex-3-en-1-ol, [(Z)-hex-3-enyl]acetate, 4,11,11-trimethyl-8-methylene-bicyclo[7.2.0]undec-4-ene, methyl-2,4-dihydroxy-3,6-dimethyl-benzoate, allyl (cyclohexyloxy)acetate, 3,3,5-trimethylcyclohexyl acetate, 1-methyl-4-(4-methylpent-3-enyl)cyclohex-3-ene-1-carbaldehyde, undec-10-enal, 5-isopropenyl-2-methyl-cyclohex-2-en-1-one, 2,4,4,7-tetramethyloct-6-en-3-one, 4-allyl-2-methoxy-phenol, 3a,4,5,6,7,7a-hexahydro-4,7-methanoinden-6-yl acetate, 2,6-dimethylhept-5-enal, 2-ethoxy-4-(methoxymethyl)phenol, methyl 2-nonynoate, (4-tert-butylcyclohexyl) acetate, 5-methylheptan-3-one oxime, (E)-4-methyldec-3-en-5-ol and mixtures thereof.

One preferred embodiment of the present invention relates a fragrance composition for conveying a positive mood, preferably an attractive mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-trimethyl-benzenepropanol, methyl-2-aminobenzoate, (E)-4-(2,6,6-trimethylcyclohex-2-en-1-yl)but-3-en-2-one, methyl 2-[(7-hydroxy-3,7-dimethyloctylidene)amino]benzoate, 2,6-dimethylheptan-2-ol, 5-hexyl-5-methyl-tetrahydrofuran-2-one, 3,7-dimethylocta-1,6-dien-3-ol, 4-(propan-2-yl)cyclohexyl]methanol, 3-(4-methoxyphenyl)-2-methyl-propanal and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-tetramethyldodecahydronaphtho[2,1-b]furan, 1,1,2,3,3,-pentamethyl-2,5,6,7-tetrahydroinden-4-one, 1,4-dioxacycloheptadecane-5,17-dione, 4,6,6,7,8,8-hexamethyl-1,3,4,7-tetrahydrocyclopenta[g]isochromene, oxacyclohexadecen-2-one (mixture of isomers), pentadecan-15-olide 8-methoxy-2,6,6,8-tetramethyl-tricyclo[5.3.1.01,5]undecane, [2-[1-(3,3-dimethylcyclohexyl)ethoxy]-2-methyl-propyl]propanoate, (E)-3-methyl-4-(2,6,6-trimethylcyclohex-2-en-1-yl)but-3-en-2-one, (5Z)-3-methylcyclopentadec-5-en-1-one, (2E)-2-ethyl-4-(2,2,3)-trimethylcyclopent-3-en-1-yl) but-2-en-1-ol, 1-[(2E,5E,9E)-2,6,10-trimethylcyclododeca-2,5,9-trien-1-yl]ethanone, 2-ethoxy-2,6,6-trimethyl-9-methylene-bicyclo[3.3.1]nonane, 4-(1-ethoxyvinyl)-3,3,5,5-tetramethyl-cyclohexanone, 1-(1,1,2,4,4,7-hexamethyltetralin-6-yl)ethanone and mixtures thereof;
and
at least one substance of group (C) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (C) consists of compounds having an earthy and/or sweet odor impression selected from the group consisting of chromen-2-one, 2-ethyl-3-hydroxy-pyran-4-one, 3-ethoxy-4-hydroxy-benzaldehyde, 4-methoxybenzaldehyde, 3-hydroxy-2-methyl-4h-pyran-4-one, 4-hydroxy-3-methoxy-benzaldehyde, (4-formyl-2-methoxy-phenyl) 2-methylpropanoate and mixtures thereof;
and
at least one essential oil and/or essence and/or absolue selected from the group consisting of bergamot oil, ginger root extract, lavender oil, vetiver oil, eucalyptus oil, grapefruit oil, jasmin absolue, lime oil, orange flower absolue, orange oil, rose absolue, sandalwood oil, tonka bean absolue.
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of allyl 3-cyclohexylpropanoate, 5-hexyltetrahydrofuran-2-one, methoxyphenylbutanone, (2E)-3,7-Dimethylocta-2,6-dienal, 5-heptyltetrahydrofuran-2-one, hexyl acetate, isopropyl tetradeconate, 6,6-dimethoxy-2,5,5-trimethylhex-2-ene, ethyl-3-oxobutanoate, ethyl-2-methylpentanoate, 5-hexyldihydro-4-methylfuran-2(3H)-one, 2-tert-butylcyclohexyl acetate, diethyl 2-methylenebutanedioate, 2-bicyclo[2.2.1]hept-5-enyl acetate, ethyl 3-oxobutanoate, linolal, mixture of diethyl (E/Z)-2-methylbut-2-enedioate and diethyl 2-methylenebutanedioate, mixture of (5-isopropenyl-2-methyl-cyclohex-2-en-1-yl) acetate and (5-isopropenyl-2-methyl-cyclohexyl) acetate, ethyl tetrahydrofuran-2-carboxylate, decanal, ethyl rac-(2r,3r)-3-methyl-3-phenyl-oxirane-2-carboxylate, allyl hexanoate, (1,1-dimethyl-2-phenyl-ethyl) acetate, 5-octyltetrahydrofuran-2-one, ethyl butanoate, ethyl 2-methylbutanoate, 3-methylbutyl acetate, ethyl 2-(2-methyl-1,3-dioxolan-2-yl)acetate, 2,4-dimethyl-4,4a,5,9b-tetrahydoindeno[1,2-d][1,3]dioxine, 2-methyl-4-propyl-1,3-oxathiane, 2,4,6-trimethyl-4-phenyl-1,3-dioxane, allyl heptanoate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 7-methyl-1,5-benzodioxepin-3-one, 5-isopropenyl-2-methyl-cyclohex-2-en-1-one, 2,4,4,7-tetramethyloct-6-en-3-one, 3a,4,5,6,7,7a-hexahydro-4,7-methanoinden-6-yl acetate, (e)-4-methyldec-3-en-5-oL and mixtures thereof.

Yet another preferred embodiment relates to a fragrance composition according to the invention for conveying a positive mood, preferably a fresh mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-trimethyl-benzenepropanol, 3-(4-ethylphenyl)-2,2-dimethylpropanal, (2E)-3,7-dimethylocta-2,6-dien-1-ol, methyl-2-[rac-(1S,2S)-3-oxo-2-pentyl-cyclopentyl]acetate, 3-(1,3-benzodioxol-5-yl)-2-methyl-propanal, (+)-3-(4-isopropylcyclohexen-1-yl)propanal, 1-phenylethyl acetate, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 3,7-dimethylocta-1,6-dien-3-ol, 4-(propan-2-yl)cyclohexyl]methanol, 4-(4-methylpent-3-enyl)cyclohex-3-ene-1-carbaldehyde, 3-(4-methoxyphenyl)-2-methyl-propanal and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-tetramethyldodecahydronaphtho[2,1-b]furan, [2-[1-(3,3-dimethylcyclohexyl)ethoxy]-2-methyl-propyl]propanoate, 1-[(2E,5E,9E)-2,6,10-trimethylcyclododeca-2,5,9-trien-1-yl]ethanone, oxacycloheptadec-10-en-2-one, 2-ethoxy-2,6,6-trimethyl-9-methylene-bicyclo[3.3.1]nonane, 1-(1,1,2,4,4,7-hexamethyltetralin-6-yl)ethanone and mixtures thereof;
and
at least one essential oil and/or essence and/or absolue selected from the group consisting of bergamot oil, ginger root extract, lavender oil, vetiver oil, artemisia oil, hemp essence, lemon oil, cardamom oil, camphor oil, eucalyptus oil, grapefruit oil, jasmin absolue, lime oil, orange oil, rose absolue, sage oil and sandalwood oil,
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of allyl 3-cyclohexylpropanoate, (2E)-3,7-dimethylocta-2,6-dienal, 5-hexyltetrahydrofuran-2-one, Methoxyphenylbutanone, 6,6-Dimethoxy-2,5,5-trimethylhex-2-ene, isopropyl tetradeconate, ethyl 3-oxobutanoate, ethyl-2-methylpentanoate, 2,4-dimethyl-4,4a,5,9b-tetrahydoindeno[1,2-d][1,3]dioxine, allyl heptanoate and mixtures thereof
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 7-methyl-1,5-benzodioxepin-3-one, 5-isopropenyl-2-methyl-cyclohex-2-en-1-one, 2,4,4,7-tetramethyloct-6-en-3-one, methyl 2-nonynoate, (4-tert-butylcyclohexyl) acetate, (E)-4-methyldec-3-en-5-ol and mixtures thereof.

Another preferred embodiment relates to a fragrance composition according to the invention for conveying a positive mood, preferably an energizing mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 2,6-dimethyloct-7-en-2-ol, (6E)-3,7-dimethylnona-1,6-dien-3-ol, 3-(4-ethylphenyl)-2,2-dimethylpropanal, 3-(1,3-benzodioxol-5-yl)-2-methyl-propanal, methyl 2-[(7-hydroxy-3,7-dimethyloctylidene)amino]benzoate, 2,6-dimethylheptan-2-ol, 3,7-dimethylocta-1,6-dien-3-ol and mixtures thereof,
and
optionally at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from (4aR,5R,7aS,9R)-octahydro-2,2,5,8,8,9a-hexamethyl-4h-4a,9-methanoazuleno(5,6-d)-1,3-dioxole;
and
optionally at least one substance of group (C) in a total amount of between 0.01 and 35 wt.-%, preferably of between 0.2 and 20 wt.-%, especially preferably of between 0.5 and 10 wt.-%,
wherein group (C) consists of compounds having an earthy and/or sweet odor impression selected from the group consisting of 2-ethyl-3-hydroxy-pyran-4-one, 3-ethoxy-4-hydroxy-benzaldehyde, 3,6-Dimethyl-3H-benzofuran-2-one, 1-methoxy-4-[(E)-prop-1-enyl]benzene, 3-hydroxy-2-methyl-4h-pyran-4-one, 4-hydroxy-3-methoxy-benzaldehyde, (4-formyl-2-methoxy-phenyl) 2-methylpropanoate and mixtures thereof;
and
at least one essential oil and/or essence and/or absolue selected from the group consisting of bergamot oil, ginger root extract, artemisia oil, cinnamon bark oil, elemi oil, ginger oil, lemon oil, lemongrass oil, longonza oil, mandarin oil, black pepper oil, red berry extract, camphor oil, cardamom oil, cypress oil, eucalyptus oil, grapefruit oil, lime oil, olibanum oil, orange oil, patchouli essence, rose absolue, rosemary oil, thyme oil and tuberose absolue; optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of (2E)-3,7-Dimethylocta-2,6-dienal, 5-hexyltetrahydrofuran-2-one, methoxyphenylbutanone, 6,6-Dimethoxy-2,5,5-trimethylhex-2-ene, hexyl acetate, decanal, ethyl butanoate, 3-methylbutyl acetate, 2,4-dimethyl-4,4a,5,9b-tetrahydoindeno[1,2-d][1,3]dioxine, 2-methyl-4-propyl-1,3-oxathiane, allyl heptanoate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 7-methyl-1,5-benzodioxepin-3-one, 1-methyl-4-(4-methylpent-3-enyl)cyclohex-3-ene-1-carbaldehyde, 2,4-Dimethylcyclohex-3-ene-1-carbaldehyde, undec-10-enal, 5-isopropenyl-2-methyl-cyclohex-2-en-1-one, 2,4,4,7-tetramethyloct-6-en-3-one, 4-allyl-2-methoxy-phenol, 2,6-dimethylhept-5-enal, (E)-4-methyldec-3-en-5-ol and mixtures thereof.

One preferred embodiment relates to a fragrance composition according to the invention for conveying a positive mood, preferably a joyful mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 7-hydroxy-3,7-dimethyl-octanal, (6E)-3,7-dimethylnona-1,6-dien-3-ol, beta,beta-3-Trimethyl-benzenepropanol, methyl-2-aminobenzoate, (E)-4-(2,6,6-trimethylcyclohex-2-en-1-yl)but-3-en-2-one, benzyl acetate, 3-(4-ethylphenyl)-2,2-dimethylpropanal, (2E)-3,7-dimethylocta-2,6-dien-1-ol, 3-(1,3-benzodioxol-5-yl)-2-methyl-propanal, [(Z)-hex-3-enyl]-2-hydroxybenzoate, (E)-4-(2,6,6-trimethylcyclohexen-1-yl)but-3-en-2-one, (+)-3-(4-Isopropylcyclohexen-1-yl)propanal, methyl 2-[(7-hydroxy-3,7-dimethyloctylidene)amino]benzoate, ethyl 2,6,6-trimethylcyclohexa-1,3-diene-1-carboxylate, 3-(3-isopropylphenyl)butanal, 2-(phenylmethylidene)octanal, (E)-1-(2,4,4-trimethylcyclohexen-1-yl)but-2-en-1-one, 3,7-dimethylocta-1,6-dien-3-ol, 2-methyl-4-phenyl-butan-2-ol, 2-methyl-5-phenylpentanol, 2,2,5-trimethyl-5-pentyl-cyclopentanone, 3-(4-methoxyphenyl)-2-methyl-propanal and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, Oxacyclohexadecen-2-one (mixture of isomers), Pentadecan-15-olide, (E)-3-methyl-4-(2,6,6-trimethylcyclohex-2-en-1-yl)but-3-en-2-one, 1-[2,3,8,8-tetramethyl-1,3,4,5,6,7-hexanhydronaphthalen-2-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, (E)-2-methyl-3-phenyl-prop-2-en-1-ol, 4-(1-ethoxyvinyl)-3,3,5,5-tetramethyl-cyclohexanone, (E)-3,4,5,6,6-pentamethylhept-3-en-2-one, 1-(1,1,2,4,4,7-hexamethyltetralin-6-yl)ethanone and mixtures thereof;
and
at least one substance of group (C) in a total amount of between 0.01 and 35 wt.-%, preferably of between 0.2 and 20 wt.-%, especially preferably of between 0.5 and 10 wt.-%,
wherein group (C) consists of compounds having an earthy and/or sweet odor impression selected from the group consisting of chromen-2-one, 2-ethyl-3-hydroxy-pyran-4-one, 3-ethoxy-4-hydroxy-benzaldehyde, 2-Methyl-5-propyl-cyclohex-2-en-1-one, 3,6-Dimethyl-3H-benzofuran-2-one, 1,3-benzodioxole-5-carbaldehyde, 5-pentyltetrahydrofuran-2-one, 1-methoxy-4-[(e)-prop-1-enyl]benzene, 4-methoxybenzaldehyde, 4-hydroxy-2,5-dimethylfuran-3-one, 3-hydroxy-2-methyl-4h-pyran-4-one, 4-hydroxy-3-methoxy-benzaldehyde, (4-formyl-2-methoxy-phenyl) 2-methylpropanoate and mixtures thereof;
and
at least one essential oil and/or essence and/or absolue selected from the group consisting of bergamot oil, lavender oil, vetiver oil, Artemisia oil, artichoke essence, elemi oil, ginger oil, lemon oil, lemongrass oil, mandarin oil, black pepper oil, red berry extract, clove essence, vanilla absolue, ylang ylang oil, benzaldehyde, cardamom oil, cypress oil, eucalyptus oil, geranium oil, grapefruit oil, jasmin absolue, lime oil, olibanum oil, range flower absolue, orange oil, rose absolue, rosemary oil, sage oil, sandalwood oil, thyme oil and tonka bean absolue;
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of allyl 3-cyclohexylpropanoate, (2E)-3,7-Dimethylocta-2,6-dienal, 5-hexyltetrahydrofuran-2-one, Methoxyphenylbutanone, 5-heptyltetrahydrofuran-2-one, 6,6-Dimethoxy-2,5,5-trimethylhex-2-ene, hexyl acetate, isopropyl tetradeconate, 2-tert-Butylcyclohexyl acetate, 5-Hexyldihydro-4-methylfuran-2(3H)-one, ethyl-2-methylpentanoate, mixture of diethyl (E/Z)-2-methylbut-2-enedioate and diethyl 2-methylenebutanedioate, ethyl rac-(2R,3R)-3-methyl-3-phenyl-oxirane-2-carboxylate, allyl hexanoate, 3-methylbutyl acetate, 2,4-dimethyl-4,4a,5,9b-tetrahydoindeno[1,2-d][1,3]dioxine, allyl heptanoate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting 7-methyl-1,5-benzodioxepin-3-one, Hex-5-enyl 2-m,ethylbutanoate, [(Z)-hex-3-enyl]acetate, (Z)-Hex-3-en-1-yl methyl carbonate, 5-isopropenyl-2-methyl-cyclohex-2-en-1-one, 2,4,4,7-tetramethyloct-6-en-3-one, 3a,4,5,6,7,7a-hexahydro-4,7-methanoinden-6-yl acetate, (E)-4-methyldec-3-en-5-ol of and mixtures thereof.

Yet another preferred embodiment relates to a fragrance composition according to the invention for conveying a positive mood, preferably a relaxing mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 3-(4-tert-butylphenyl)propanal, 2,6-dimethyloct-7-en-2-ol, 7-hydroxy-3,7-dimethyl-octanal, 3,7-dimethyloct-6-en-1-ol, 3-(4-isopropylphenyl)-2-methyl-propanal, beta,beta-3-Trimethyl-benzenepropanol, methyl-2-aminobenzoate, (E)-4-(2,6,6-trimethylcyclohex-2-en-1-yl)but-3-en-2-one, benzyl 2-hydroxybenzoate, methyl-2-[RAC-(1S,2S)-3-oxo-2-pentyl-cyclopentyl]acetate, (+)-3-(4-Isopropylcyclohexen-1-yl)propanal, (1,5-dimethyl-1-vinyl-hex-4-enyl)acetate, 2-phenylethanol, 4-Isopentylcyclohexanol, methyl 2-[(7-hydroxy-3,7-dimethyloctylidene)amino]benzoate, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 2,3-dihydro-1,1-dimethyl-1h-indene-ar-propanal, (2e)-2-methyl-6-methylene-octa-2,7-dien-1-ol, 5-hexyl-5-methyl-tetrahydrofuran-2-one, 4-(propan-2-yl)cyclohexyl]methanol, 3-methyl-5-phenylpentan-1-ol, 2-methyl-5-phenylpentanol, 3-(4-methoxyphenyl)-2-methyl-propanal and mixtures thereof, and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, 1,4-dioxacycloheptadecane-5,17-dione, 4,6,6,7,8,8-hexamethyl-1,3,4,7-tetrahydrocyclopenta[G]isochromene, Oxacyclohexadecen-2-one (mixture of isomers), Pentadecan-15-olide, 8-methoxy-2,6,6,8-tetramethyl-tricyclo[5.3.1.01,5]undecane, [2-[1-(3,3-dimethylcyclohexyl)ethoxy]-2-methyl-propyl]propanoate, (E)-3-methyl-4-(2,6,6-trimethylcyclohex-2-en-1-yl)but-3-en-2-one, (5Z)-3-methylcyclopentadec-5-en-1-one, (2E)-2-Ethyl-4-(2,2,3)-trimethylcyclopent-3-en-1-yl) but-2-en-1-ol, 1-[(2E,5E,9E)-2,6,10-trimethylcyclododeca-2,5,9-trien-1-yl]ethanone, oxacycloheptadec-10-en-2-one, 2-ethoxy-2,6,6-trimethyl-9-methylene-bicyclo[3.3.1]nonane, (E)-2-methyl-3-phenyl-prop-2-en-1-ol, rac-(E,2R,3R)-3-methyl-5-(2,2,3-trimethylcyclopent-3-en-1-yl)pent-4-en-2-ol, 4-(1,7,7-trimethyl-2-bucyclo[3.1.1]heptanyl)ccyclohexan-1-ol, 4-(1-ethoxyvinyl)-3,3,5,5-tetramethyl-cyclohexanone, 1-(2,6,6,8-tetramethyl-9-tricyclo[5.3.1.01,5]undec-8-enyl)ethanone, 1-(1,1,2,4,4,7-hexamethyltetralin-6-yl)ethanone, oxacycloheptadec-10-en-2-one, DL Ambre 83 SMP N, Methyl-2,4-dihydroxy-3,6-dimethyl-benzoate, (4-tert-butylcyclohexyl) acetate and mixtures thereof;
and
at least one substance of group (C) in a total amount of between 0.01 and 99 wt.-%, preferably of between 0.01 and 35 wt.-%, preferably of between 0.2 and 20 wt.-%, especially preferably of between 0.5 and 10 wt.-%,
   wherein group (C) consists of compounds having an earthy and/or sweet odor impression selected from the group consisting of chromen-2-one, 4-methoxybenzaldehyde, 3-hydroxy-2-methyl-4H-pyran-4-one, 4,6,6,7,8,8-hexamethyl-1,3,4,7-tetrahydrocyclopenta[G]isochromene, 4-hydroxy-3-methoxy-benzaldehyde, (4-formyl-2-methoxy-phenyl) 2-methylpropanoate, [2-[1-(3,3-dimethylcyclohexyl)ethoxy]-2-methyl-propyl]propanoate, (E)-4-(2,6,6-trimethylcyclohex-2-en-1-yl)but-3-en-2-one, pentadecan-15-olide and mixtures thereof;
and
at least one essential oil and/or essence and/or absolue selected from the group consisting of lavender oil, cedarwood oil, cypress oil, eucalyptus oil, geranium oil, lime oil, orange flower absolue, rose absolue, rosemary oil, sage oil and tonka bean absolue;
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of 5-heptyltetrahydrofuran-2-one, 5-hexyldihydro-4-methylfuran-2(3h)-one, ethyl 3-oxobutanoate, 5-octyltetrahydrofuran-2-one, ethyl 2-(2-methyl-1,3-dioxolan-2-yl)acetate, gamma-dodecalactone and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 4,11,11-trimethyl-8-methylene-bicyclo[7.2.0]undec-4-ene, methyl-2,4-dihydroxy-3,6-dimethyl-benzoate, 5-isopropenyl-2-methyl-cyclohex-2-en-1-one, 3a,4,5,6,7,7a-hexahydro-4,7-methanoinden-6-yl acetate, 2-ethoxy-4-(methoxymethyl)phenol, (4-tert-butylcyclohexyl) acetate, (E)-4-methyldec-3-en-5-ol and mixtures thereof.

One preferred embodiment of the present invention relates to a fragrance composition according to the invention for conveying a positive mood, preferably a self-confident mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 3-(4-tert-butylphenyl)propanal, 7-hydroxy-3,7-dimethyl-octanal, 3,7-dimethyloct-6-en-1-ol, 3-(4-isopropylphenyl)-2-methyl-propanal, beta,beta-3-Trimethyl-benzenepropanol, (E)-4-(2,6,6-trimethylcyclohex-2-en-1-yl)but-3-en-2-one, benzyl 2-hydroxybenzoate, (+)-3-(4-Isopropylcyclohexen-1-yl)propanal, 2-phenylethanol, ethyl 2,6,6-trimethylcyclohexa-1,3-diene-1-carboxylate, 5-hexyl-5-methyl-tetrahydrofuran-2-one and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 1,1,2,3,3,-pentamethyl-2,5,6,7-tetrahydroinden-4-one, 1,4-dioxacycloheptadecane-5,17-dione, Oxacyclohexadecen-2-one (mixture of isomers), Pentadecan-15-olide, [2-[1-(3,3-dimethylcyclohexyl)ethoxy]-2-methyl-propyl]propanoate, Oxacycloheptadec-10-en-2-one, rac-(E,2R,3R)-3-methyl-5-(2,2,3-trimethylcyclopent-3-en-1-yl)pent-4-en-2-ol and mixtures thereof;
and
at least one substance of group (C) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (C) consists of compounds having an earthy and/or sweet odor impression selected from the group consisting of chromen-2-one, 4-methoxybenzaldehyde and mixtures thereof;
and
optionally at least one essential oil selected from the group consisting of lavender oil, cedarwood oil and hemp oil;
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of 5-heptyltetrahydrofuran-2-one, 5-Hexyldihydro-4-methylfuran-2(3H)-one, ethyl 2-(2-methyl-1,3-dioxolan-2-yl)acetate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 4,11,11-trimethyl-8-methylene-bicyclo[7.2.0]undec-4-ene, 2-ethoxy-4-(methoxymethyl)phenol and mixtures thereof.

Another preferred embodiment relates to a fragrance composition according to the invention for conveying a positive mood, preferably a well-being mood, in a subject, preferably a human, wherein the fragrance composition comprises or consists of:
at least one substance of group (A) in a total amount of between 0.01 and 70 wt.-%, preferably of between 0.2 and 50 wt.-%, especially preferably of between 0.5 and 35 wt.-%, moreover preferably of between 1 and 20 wt.-%,
wherein group (A) consists of compounds with a bloomy odor impression selected from the group consisting of 7-hydroxy-3,7-dimethyl-octanal, beta,beta-3-Trimethyl-benzenepropanol, (E)-4-(2,6,6-trimethylcyclohex-2-en-1-yl)but-3-en-2-one, benzyl acetate, 3-(1,3-benzodioxol-5-yl)-2-methyl-propanal, (E)-4-(2,6,6-trimethylcyclohexen-1-yl)but-3-en-2-one, (+)-3-(4-Isopropylcyclohexen-1-yl)propanal, methyl 2-[(7-hydroxy-3,7-dimethyloctylidene)amino]benzoate, ethyl 2,6,6-trimethylcyclohexa-1,3-diene-1-carboxylate, 3-(3-isopropylphenyl)butanal, 2-(phenylmethylidene)octanal, 2-methyl-4-phenyl-butan-2-ol, 2-methyl-5-phenylpentanol, 3-(4-methoxyphenyl)-2-methyl-propanal and mixtures thereof,
and
at least one substance of group (B) in a total amount of between 0.01 and 50 wt.-%, preferably of between 0.2 and 35 wt.-%, especially preferably of between 0.5 and 20 wt.-%,
wherein group (B) consists of compounds with a woody and/or balsam odor impression, selected from the group consisting of 3a,6,6,9a-Tetramethyldodecahydronaphtho[2,1-b]furan, 1,1,2,3,3,-pentamethyl-2,5,6,7-tetrahydroinden-4-one, Oxacyclohexadecen-2-one (mixture of isomers), Pentadecan-15-olide, (E)-3-methyl-4-(2,6,6-trimethylcyclohex-2-en-1-yl)but-3-en-2-one, 1-[2,3,8,8-tetramethyl-1,3,4,5,6,7-hexanhydronaphthalen-2-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, (E)-2-methyl-3-phenyl-prop-2-en-1-ol, 4-(1-ethoxyvinyl)-3,3,5,5-tetramethyl-cyclohexanone, (e)-3,4,5,6,6-pentamethylhept-3-en-2-one, 1-(1,1,2,4,4,7-hexamethyltetralin-6-yl)ethanone and mixtures thereof;
and
at least one substance of group (C) in a total amount of between 0.01 and 35 wt.-%, preferably of between 0.2 and 20 wt.-%, especially preferably of between 0.5 and 10 wt.-%,
wherein group (C) consists of compounds having an earthy and/or sweet odor impression selected from the group consisting of chromen-2-one, 2-ethyl-3-hydroxy-pyran-4-one, 3-ethoxy-4-hydroxy-benzaldehyde, 2-Methyl-5-propyl-cyclohex-2-en-1-one, 3,6-Dimethyl-3H-benzofuran-2-one, 1,3-benzodioxole-5-carbaldehyde, 5-pentyltetrahydrofuran-2-one, 1-methoxy-4-[(E)-prop-1-enyl]benzene, 4-methoxybenzaldehyde, 4-hydroxy-2,5-dimethylfuran-3-one, 3-hydroxy-2-methyl-4h-pyran-4-one, 4-hydroxy-3-methoxy-benzaldehyde, (4-formyl-2-methoxy-phenyl) 2-methylpropanoate and mixtures thereof;
and
at least one essential oil and/or essence and/or absolue selected from the group consisting of bergamot oil, lavender oil, vetiver oil, artichoke essence, elemi oil, ginger oil, lemongrass oil, mandarin oil, black pepper oil, red berry extract, vanilla absolue, ylang ylang oil, cardamom oil, eucalyptus oil, geranium oil, grapefruit oil, jasmin absolue, lime oil, olibanum oil, orange flower absolue, orange oil, rose absolue, rosemary oil, sage oil, sandalwood oil, thyme oil and tonka bean absolue;
and
optionally at least one substance of group (D) in a total amount of between 0.01 and 35 wt.-%, preferably of between 0.2 and 20 wt.-%, especially preferably of between 0.5 and 10 wt.-%,
wherein group (D) consists of compounds with a fruity odor impression, selected from the group consisting of allyl 3-cyclohexylpropanoate, (2E)-3,7-Dimethylocta-2,6-dienal, 5-hexyltetrahydrofuran-2-one, Methoxyphenylbutanone, 5-heptyltetrahydrofuran-2-one, 6,6-Dimethoxy-2,5,5-trimethylhex-2-ene, hexyl acetate, isopropyl tetradeconate, ethyl-2-methylpentanoate, ethyl rac-(2R,3R)-3-methyl-3-phenyl-oxirane-2-carboxylate, allyl hexanoate, 3-methylbutyl acetate, allyl heptanoate and mixtures thereof;
and
optionally at least one substance of group (E) in a total amount of between 0.01 and 35 wt.-%, preferably of between 0.2 and 20 wt.-%, especially preferably of between 0.5 and 10 wt.-%,
wherein group (E) consists of compounds having green and/or herbal odor impression selected from the group consisting of 7-methyl-1,5-benzodioxepin-3-one, hex-5-enyl 2-methylbutanoate, (Z)-Hex-3-en-1-yl methyl carbonate, 5-isopropenyl-2-methyl-cyclohex-2-en-1-one, 3a,4,5,6,7,7a-hexahydro-4,7-methanoinden-6-yl acetate, (E)-4-methyldec-3-en-5-ol and mixtures thereof.

Furthermore, the present invention relates to perfumed products, preferably as described above, comprising such a fragrance composition as described above.

Further preferred embodiments of uses and methods of the present invention become apparent in light of the above description, in particular regarding preferred embodiments of compositions and products according to the present invention, and vice versa.

The invention will now be described in more detail hereinafter with references to selected non-limiting examples.

### EXAMPLES

1. Sensorial and Emotional Evaluation of Fragrance Raw Materials Each fragrance was evaluated by 30 subjects. At each appointment, the subject discovered fragrances in one sequence of four fragrances and two sequences of three fragrances separated by a 10-minute break.

During those appointments, the subjects smelt the fragrances in a random way.

For each fragrance, the subject discovered each fragrance on a card strip for 10 seconds. Between each product, a rest-period of 2 minutes was carried out during which the subject smelt their own body odour by smelling their wrist and the subject drank a little bit of water as well.

The evaluation is carried out in standardized lighting conditions and under a monitored temperature (temperature 21±1.5° C.).

The different recordings are carried in technical room during talking with the technicians. he following tests were performed for more than 100 different fragrance raw materials.

The following testing procedures were used to determine one mood out of five different testing strategies. All results of the single testing protocols as described in 1.1 to 1.5 were combined and one mood per fragrance raw material or fragrance composition was determined based on the single testing results.

### 1.1 Vocal Analysis (Prosody)

Speech communication conveys more than the syntactic and semantic content of sentences. Various prosodic cues are available to speaker and listener in order to encode and decode the full spoken message. In addition to fulfilling a linguistic function such as to structure discourse and dialogue, and signal focus, prosodic cues provide information such as the speaker's gender, age and physical condition, and the speaker's view, emotion, and attitude towards the topic, the dialogue partner, or the situation.

This physical analysis of acoustic signal has already been used to successfully discriminate different emotional states, and represents reliable tool to evaluate the emotional level of subjects at a given moment.

The studied parameters are vocal intensity in dB (vocal volume), the mean fundamental frequency in Hz (pitch) and the coefficient of variation of the fundamental frequency in % (regularity of the pitch/vocal tonality).

### 1.2 Semantic Analysis (Verbatim)

The analysis of the verbatim corresponds to a quantitative approach (statistic) of the word content of the subject's answers to the questions asked by the technician during the study. This type of approach is traditional method of the social and human sciences in which we put in evidence the frequency of the different verbal occurrences in order to determinate the statistic importance. These analyses may be eventually made for associations between items, especially as part of the study concerned by the structure of the language.

### 1.3 Psychometric Scale (Geneva Emotion Wheel (GEW))

Emotions can be defined as multi-componential, including subjective feeling, appraisals, reactions in the service of action preparation and expressions, action tendencies (including expressions), and regulation (compare Scherer, 2005; Frijda, 2007). A central component of emotions, the “feeling component,” is inherently subjective and can only be assessed with self-report measures, such as the Geneva Emotion Wheel (GEW). The GEW consists of discrete emotion terms corresponding to emotion families that are systematically aligned in a circle. Underlying the alignment of the emotion terms are the two dimensions valence (negative to positive) and control (low to high), separating the emotions in four quadrants: Negative/low control, negative/high control, positive/low control, and positive/high control.

The subjects are filling the questionnaire after each fragrance discovery individually.

### 1.4 Implicit Response Test (IRT)

Implicit response tests (IRTs) are a family of tests and assess the subjects' immediate, “gut instinct” responses the fragrance raw materials.

The alleged underlying processes are based on how neurosciences describe the way our brain processes information. Indeed, our brain processes information through two systems: System 2, which is slow and processes conscious information, and System 1, which is fast and processes non-conscious information. IRTs are designed to measure the System 1, revealing hidden information that interviews, questionnaires, or focus groups cannot.

The IRT tests are performed at a computer, where the following words appear on the computer screen:
Energizing
Relaxing
Attractive
Sensual
Joyful
Protective
Confident
Fresh
Well-being

The subject has to click on the screen with the computer mouse to indicate their accordance or not of the word with the tested fragrance. In addition, the times to click the button are recorded.

### 1.5 Emotional Characterization: Emochar™

Emochar™ is a system of emotional characterization, scoring 7 main emotional dimensions from 0 to 10:
Stimulation: focus, attention, and awakening.
Relaxation: serenity, relaxation and appeasing.
Sensual: arousing, sexy and erotic.
Pleasure: liking, “gourmand” and satisfaction.
Tenderness: gentleness and softness.
Joy: excitement, fun and enthusiasm.
Self-esteem: confidence and assurance

### 1.6 Actimood Scoring

Actimood scores are based on a correlation of EmoChar™ and IRT values. The scores are classified on a scale from 0 (low impact on subject's mood) to 3 (high impact on subject's mood).

Each score is obtained according to an algorithm, mixing verbal and non-verbal responses of volunteers to a specific product, or stimulus in a given context. The algorithm was developed from extensive field data, and in accordance with scientific literature and validated biobehavioral patterns associated to various emotional states.

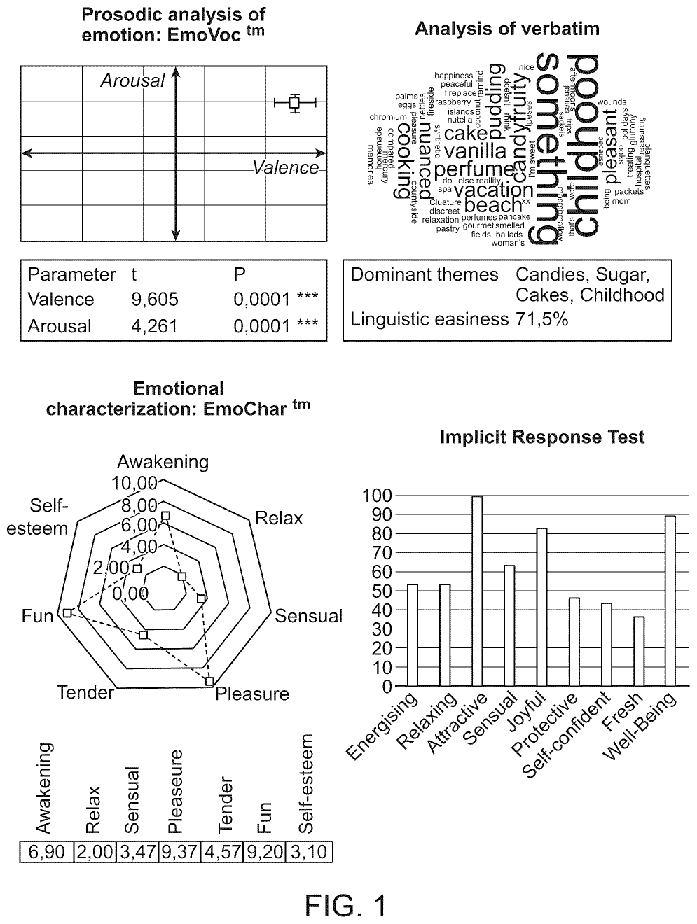

The results for the fragrance raw material 2-ethyl-3-hydroxy-pyran-4-one as being an example for a fragrance raw material conveying an attractive mood are displayed in FIG. 1.

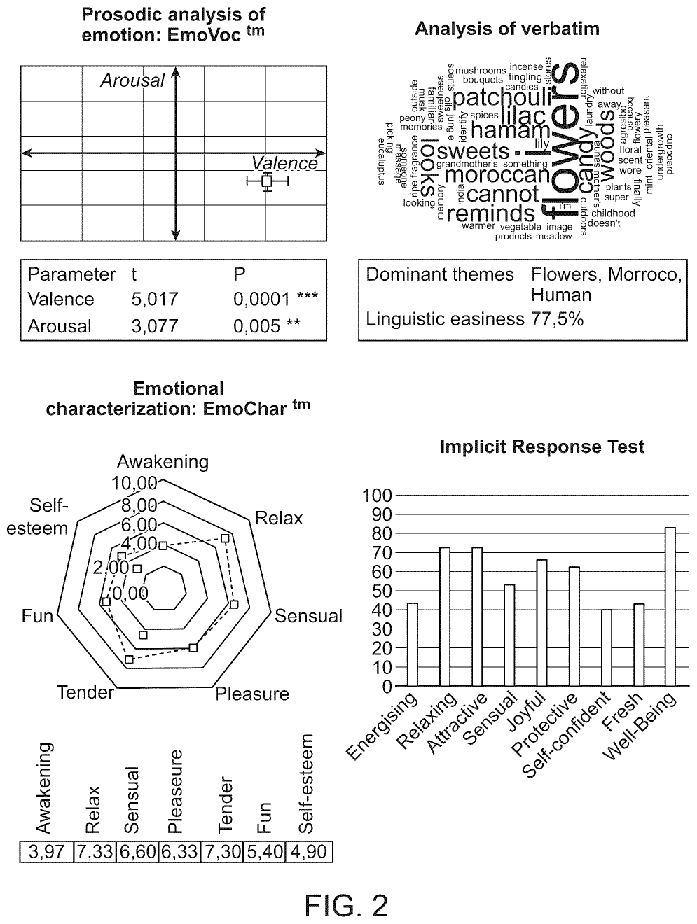

The results for the fragrance raw material 1,1,2,3,3,-pentamethyl-2,5,6,7-tetrahydroinden-4-one as being an example for a fragrance raw material conveying a protective mood, are displayed in FIG. 2.

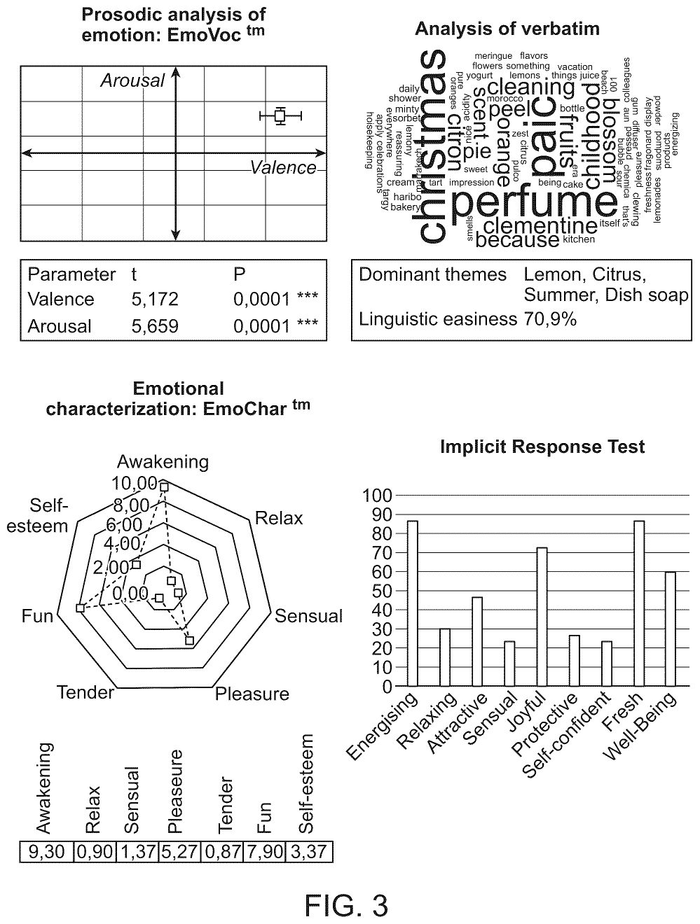

The results for the fragrance raw materials lemon essence oil as being an example for a fragrance raw material conveying a fresh mood are displayed in FIG. 3.

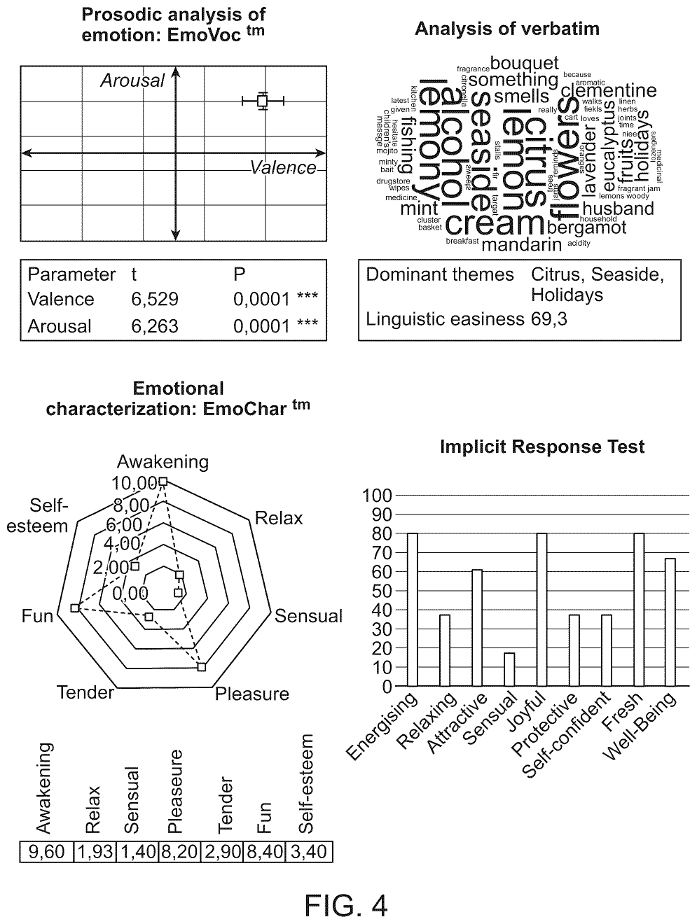

The results for the fragrance raw materials bergamot essence oil as being an example for a fragrance raw material conveying an energizing mood are displayed in FIG. 4.

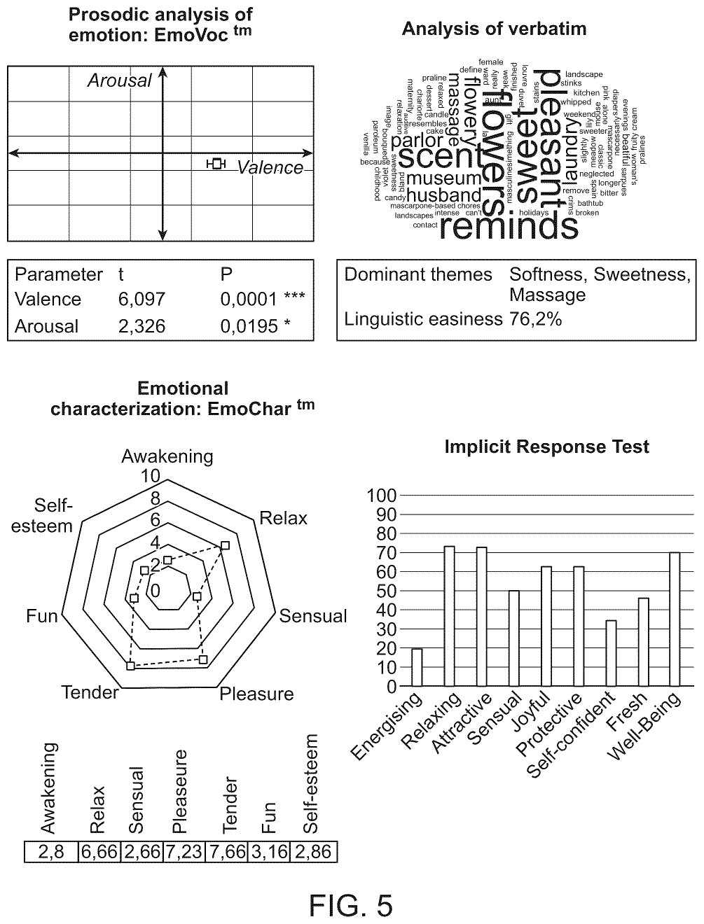

The results for the fragrance raw materials oxacyclohexadecen-2-one as being an example for a fragrance raw material conveying a relaxing mood are displayed in FIG. 5.

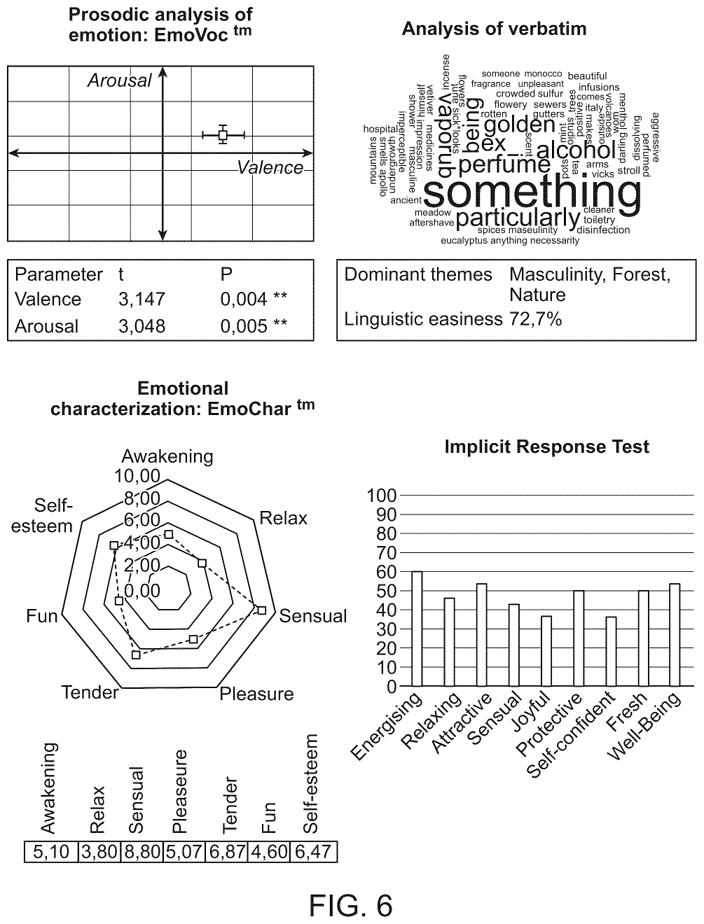

The results for the fragrance raw materials 8-methoxy-2,6,6,8-tetramethyl-tricyclo[5.3.1.01,5]undecane as being an example for a fragrance raw material conveying a sensual mood, are displayed in FIG. 6.

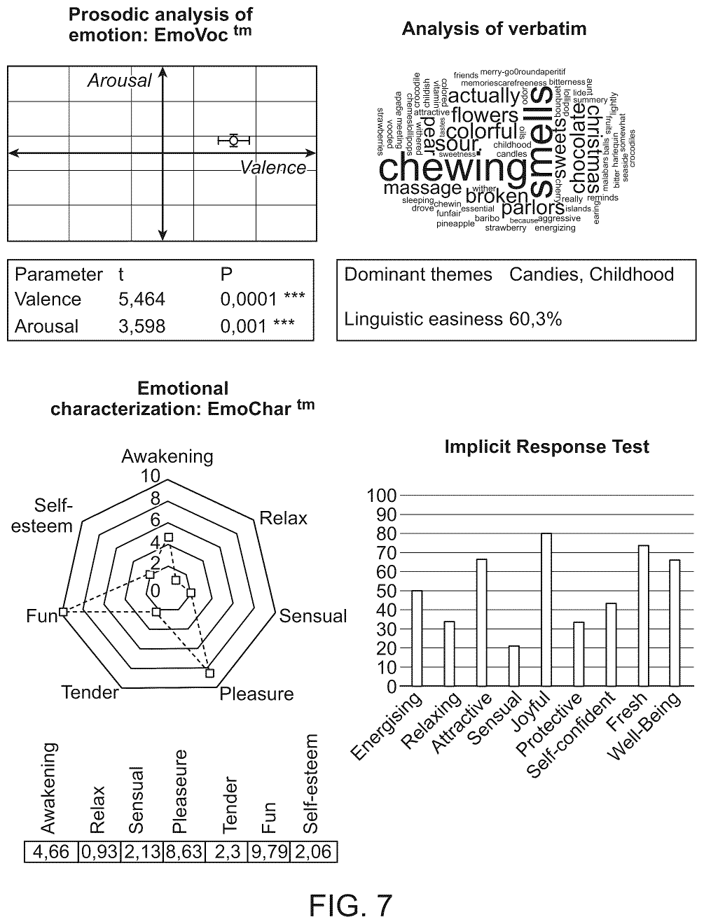

The results for the fragrance raw materials allyl 3-cyclohexylpropanoate as being an example for a fragrance raw material conveying a joyful mood, are displayed in FIG. 7.

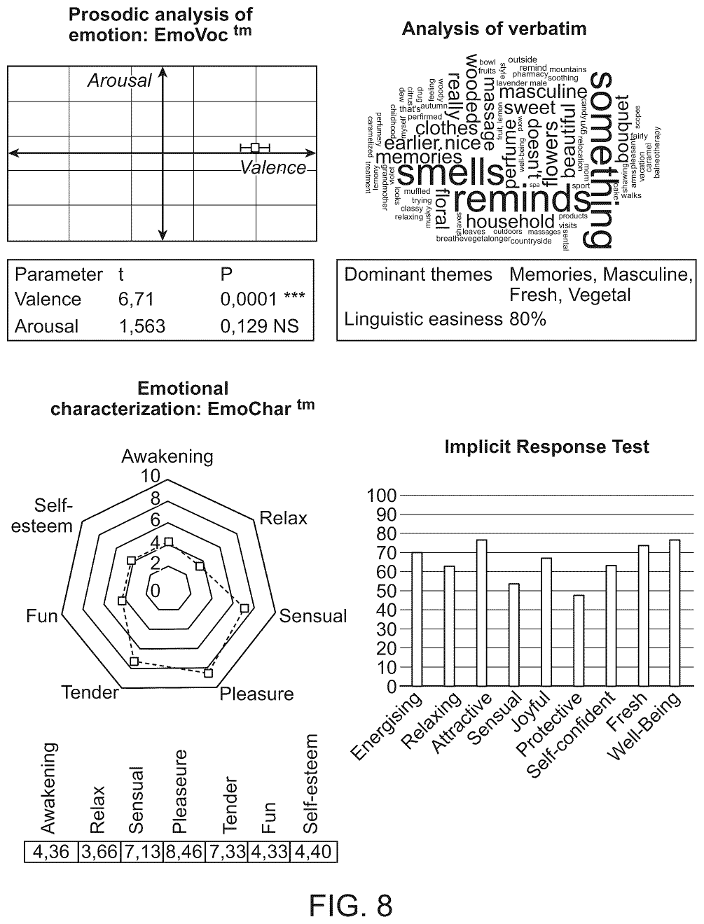

The results for the fragrance raw materials 3a,6,6,9a-Tetramethyldodecahydronaphtho[2,1-b]furan as being an example for a fragrance raw material conveying a self-confident mood, are displayed in FIG. 8.

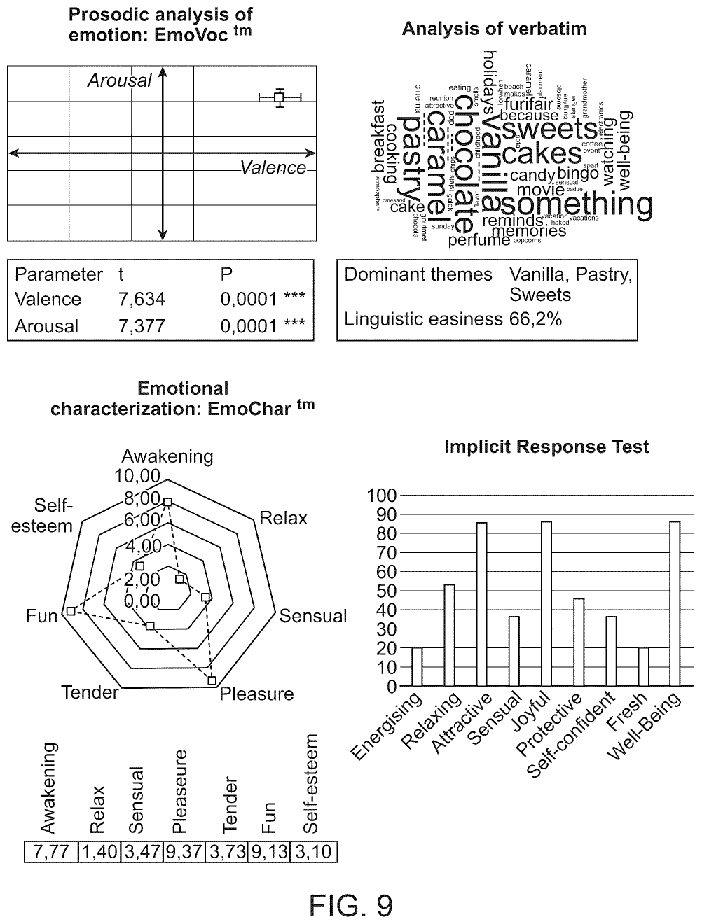

The results for the fragrance raw material 3-ethoxy-4-hydroxy-benzaldehyde as being an example for a fragrance raw material conveying a well-being mood, are displayed in FIG. 9.

### 2. Sensorial and Emotional Evaluation of Fragrance Compositions

Fragrance compositions were designed by combining the tested fragrance raw materials and the compositions were tested in 30 different subjects to evaluate the emotional impression conveyed by the fragrance composition as described above for the fragrance raw materials.

### 2.1 Fragrance Composition Conveying a Fresh Mood

The fragrance composition consists of the ingredients listed in Table 1:

| | IRT | Amount |
|---|---|---|
| Ingredient | Score | [wt.-%] |
| 3-(1,3-benzodioxol-5-yl)-2-methyl-propanal | 742 | 33.33 |
| Bergamot essential oil | 2201 | 22.22 |
| (6E)-3,7-dimethylnona-1,6-dien-3-ol | 1682 | 22.22 |
| 3-(4-isopropylphenyl)-2-methyl-propanal | 1005 | 6.67 |
| 6,6-Dimethoxy-2,5,5-trimethylhex-2-ene | 1763 | 5.56 |
| 3-(4-ethylphenyl)-2,2-dimethylpropanal | — | 3.33 |
| (2E)-3,7-Dimethylocta-2,6-dienal | 2393 | 1.67 |
| Lavender essence oil | 1357 | 1.67 |
| 3a,6,6,9a-Tetramethyldodecahydronaphtho[2,1-b]furan | 1643 | 1.11 |
| 7-methyl-1,5-benzodioxepin-3-one | 2830 | 1.11 |
| (+)-3-(4-Isopropylcyclohexen-1-yl)propanal | — | 1.11 |

The targeted mood was confirmed by subjects from France and China, wherein the Chinese subjects had a slight sensual and joyful side-impression.

### 2.2 Fragrance Composition Conveying an Attractive Mood

The fragrance composition consists of the ingredients listed in Table 2:

| | IRT | Amount |
|---|---|---|
| Ingredient | Score | [wt.-%] |
| (6E)-3,7-dimethylnona-1,6-dien-3-ol | 565 | 24.29 |
| 1,4-dioxacycloheptadecane-5,17-dione | 1177 | 17.14 |
| 1,1,2,3,3,-pentamethyl-2,5,6,7-tetrahydroinden-4-one | 1469 | 14.29 |
| 3a,6,6,9a-Tetramethyldodecahydronaphtho[2,1-b]furan | 1921 | 7.14 |
| 2,6-dimethyloct-7-en-2-ol | 1599 | 7.14 |
| 3-ethoxy-4-hydroxy-benzaldehyde | 2786 | 7.14 |
| 7-hydroxy-3,7-dimethyl-octanal | 1283 | 7.14 |
| 2-ethyl-3-hydroxy-pyran-4-one | 3505 | 5.71 |
| Oxacyclohexadecen-2-one | 1815 | 2.86 |
| Pentadecan-15-olide | 1944 | 2.86 |
| 3-(4-tert-butylphenyl)propanal | 1045 | 1.43 |
| chromen-2-one | 1347 | 1.43 |
| Lavender essential oil | 505 | 1.43 |

The targeted mood was confirmed by subjects from France and China, wherein both groups of subjects perceived an additional self-confident mood.

### 2.3 Fragrance Composition Conveying a Relaxing Mood

The following fragrance compositions convey a relaxing mood and have also benefits on the sleep.

### 2.3.1 Composition 1 (Relaxing and Sleep Enhancing)

TABLE 3

| Name | Chemical name | Group | [wt.-%] | Actimood score |
|---|---|---|---|---|
| AMBRETTOLIDE HC SUPREME | Oxacycloheptadec-10-en-2-one | B | 0.57% | 2 |
| DL AMBRE 83 SMP N | Mixture of ingredients | B | 0.57% | 2 |
| DODECALACTONE GAMMA | gamma-Dodecalactone | D | 1.71% | 1 |
| 10% DPG | | | | |
| ETHYLENE BRASSYLATE | 1,4-dioxacycloheptadecane-5,17- | C | 71.14% | 2 |
| | dione | | | |
| EVERNYL 10% DPG | Methyl-2,4-dihydroxy-3,6- | B | 0.86% | 1 |
| | dimethyl-benzoate | | | |
| HEDIONE | methyl-2-[RAC-(1S,2S)-3-oxo-2- | A | 0.29% | 2 |
| | pentyl-cyclopentyl]acetate | | | |
| HELVETOLIDE | [2-[1-(3,3- | C | 4.29% | 3 |
| | dimethylcyclohexyl)ethoxy]-2- | | | |
| | methyl-propyl]propanoate | | | |
| LAVANDIN GROSSO BM | Lavender oil | Essential oil | 1.43% | 1 |
| LILIAL REPLACER | Mixture of ingredients | Essential oil | 17.14% | 1 |
| ORYCLON HC | (4-tert-butylcyclohexyl) acetate | B | 0.57% | 1 |
| PHENYLETHYL ALCOHOL BA | 2-phenylethanol | A | 1.14% | 2 |
| FREE | | | | |
| SYMROSE ® | 4-Isopentylcyclohexanol | A | 0.29% | 2 |

### 2.3.2 Composition 2 (Relaxing)

TABLE 4

| Name | Chemical name | Group | [wt.-%] | Actimood score |
|---|---|---|---|---|
| BOURGEONAL | 3-(4-tert-butylphenyl)propanal | A | 2.15% | 3 |
| CARYOPHYLLENE NAT. | 4,11,11-trimethyl-8-methylene- | E | 0.15% | 3 |
| RECT. | bicyclo[7.2.0]undec-4-ene | | | |
| COUMARIN | chromen-2-one | C | 3.00% | 3 |
| GALAXOLIDE PURE | 4,6,6,7,8,8-hexamethyl-1,3,4,7- | C | 68.20% | 3 |
| | tetrahydrocyclopenta[G]isochromene | | | |
| HELVETOLIDE | [2-[1-(3,3- | C | 8.30% | 3 |
| | dimethylcyclohexyl)ethoxy]-2- | | | |
| | methyl-propyl]propanoate | | | |
| IONONE ALPHA | (E)-4-(2,6,6-trimethylcyclohex-2-en- | A | 3.00% | 3 |
| | 1-yl)but-3-en-2-one | | | |
| MACROLIDE ® SUPRA | Pentadecan-15-olide | C | 15.20% | 3 |

The above listed fragrance composition was able to convey a relaxing mood and improve the subject's sleep quality.

### 2.4 Fragrance Composition Conveying a Protective Mood

TABLE 5

| Name | Chemical name | Group | [wt.-%] | Actimood score |
|---|---|---|---|---|
| CASHMERAN | 1,1,2,3,3,-pentamethyl-2,5,6,7- | B | 6.67% | 2 |
| | tetrahydroinden-4-one | | | |
| ETHYL | 3-ethoxy-4-hydroxy-benzaldehyde | C | 0.67% | 1 |
| VANILLIN | | | | |
| GALAXOLIDE | 4,6,6,7,8,8-hexamethyl-1,3,4,7- | C | 50.00% | 2 |
| PURE | tetrahydrocyclopenta[G]isochromene | | | |
| GLOBALIDE ® | Oxacyclohexadecen-2-one | C | 26.67% | 2 |
| HELVETOLIDE | [2-[1-(3,3- | C | 6.67% | 2 |
| | dimethylcyclohexyl)ethoxy]-2- | | | |
| | methyl-propyl]propanoate | | | |
| LAVENDER | Lavender oil | Essential oil | 3.33% | 2 |
| OIL MONT | | | | |
| BLANC | | | | |
| 40/42% NEW | | | | |
| MACROLIDE ® | Pentadecan-15-olide | C | 2.67% | 2 |
| SUPRA | | | | |
| TRIMOFIX O | 1-[(2E,5E,9E)-2,6,10- | B | 3.33% | 2 |
| | trimethylcyclododeca-2,5,9-trien-1- | | | |
| | yl]ethanone | | | |

The above listed fragrance composition was able to convey a protective mood.

### 2.5 Fragrance Composition Conveying a Joyful Mood

TABLE 6

| Name | Chemical name | Group | [wt.-%] | Actimood score |
|---|---|---|---|---|
| ALLYL | allyl 3-cyclohexylpropanoate | D | 1.00% | 3 |
| CYCLOHEXYL | | | | |
| PROPIONATE | | | | |
| AMBROXIDE | 3a,6,6,9a- | B | 0.50% | 3 |
| | Tetramethyldodecahydronaphtho[2,1- | | | |
| | b]furan | | | |
| APRIFLOREN ® | 5-Hexyldihydro-4-methylfuran-2(3H)- | D | 1.00% | 2 |
| | one | | | |
| BERGAMOT | Bergamot oil | Essence oil | 10.00% | 3 |
| ECO ESSENCE | | | | |
| (E0636) W/O | | | | |
| MYRCENE | | | | |
| CALONE 10% | 7-methyl-1,5-benzodioxepin-3-one | A | 5.00% | 3 |
| DPG | | | | |
| DIPROPYLENE | Dipropylene glycol | — | 30.80% | |
| GLYCOL | | | | |
| ETHYL | (6E)-3,7-dimethylnona-1,6-dien-3-ol | A | 5.00% | 2 |
| LINALOOL | | | | |
| ETHYL | 2-ethyl-3-hydroxy-pyran-4-one | C | 1.00% | 3 |
| MALTOL | | | | |
| FRAMBINON ® | Methoxyphenylbutanone | D | 1.00% | 3 |
| GINGER OIL | ginger oil | Essence oil | 0.50% | 1 |
| MADAG. | | | | |
| HEDIONE | Methyldihydrojasmonat | Other | 20.00% | 0 |
| HEXYL | hexyl acetate | D | 1.00% | 2 |
| ACETATE | | | | |
| IONONE BETA | (E)-4-(2,6,6-trimethylcyclohexen-1- | A | 2.00% | 3 |
| | yl)but-3-en-2-one | | | |
| ISO E SUPER | 1-[2,3,8,8-tetramethyl-1,3,4,5,6,7- | B | 15.00% | 2 |
| | hexanhydronaphthalen-2-yl]ethanone | | | |
| LEAFOVERT ® | (Z)-Hex-3-en-1-yl methyl carbonate | E | 0.20% | 3 |
| LEMON OIL | Lemon oil | Essence oil | 5.00% | 2 |
| SFUM. DECOL. | | | | |
| MANZANATE | ethyl-2-methylpentanoate | D | 1.00% | 3 |

The above listed fragrance composition was able to convey a joyful mood.

### 2.6 Fragrance Composition Conveying an Energizing Mood

TABLE 7

| Name | Chemical name | Group | [wt.-%] | Actimood score |
|---|---|---|---|---|
| AMAROCIT ® | 6,6-Dimethoxy-2,5,5-trimethylhex- | D | 18.00% | 3 |
| | 2-ene | | | |
| BERGAMOT | Bergamot oil | Essence oil | 10.00% | 3 |
| ECO ESSENCE | | | | |
| (E0636) W/O | | | | |
| MYRCENE | | | | |
| CALONE | 7-methyl-1,5-benzodioxepin-3-one | D | 1.50% | 3 |
| CITRAL FF | (2E)-3,7-Dimethylocta-2,6-dienal | D | 5.00% | 3 |
| DIHYDRO | 2,6-dimethyloct-7-en-2-ol | A | 20.00% | 3 |
| MYRCENOL | | | | |
| ETHYL | (6E)-3,7-dimethylnona-1,6-dien-3-ol | A | 34.50% | 1 |
| LINALOOL | | | | |
| FLORAZON | 3-(4-ethylphenyl)-2,2- | A | 4.00% | |
| | dimethylpropanal | | | |
| HEXYL | hexyl acetate | D | 4.00% | 1 |
| ACETATE | | | | |
| LEMONGRASS | Lemon grass oil | Essence oil | 1.00% | 2 |
| OIL RED | | | | |
| MADA | | | | |
| VERTOCITRAL | 2,4-Dimethylcyclohex-3-ene-1- | A | 2.00% | 1 |
| | carbaldehyde | | | |

The above listed fragrance composition was able to convey an energizing mood.

### 2.7 Fragrance Composition Conveying a Sensual Mood

TABLE 8

| Name | Chemical name | Group | [wt.-%] | Actimood score |
|---|---|---|---|---|
| AMBROXIDE | 3a,6,6,9a- | B | 1.20% | 2 |
| | Tetramethyldodecahydronaphtho[2,1- | | | |
| | b]furan | | | |
| CEDRAMBER | 8-methoxy-2,6,6,8-tetramethyl- | B | 10.00% | 3 |
| | tricyclo[5.3.1.01,5]undecane | | | |
| DIHYDRO | 2,6-dimethyloct-7-en-2-ol | A | 12.00% | 3 |
| MYRCENOL | | | | |
| ETHYLENE | 1,4-dioxacycloheptadecane-5,17- | C | 50.00% | 3 |
| BRASSYLATE | dione | | | |
| FRUCTATE ® | Hex-5-enyl 2-methylbutanoate | E | 4.00% | 0 |
| ISO E SUPER | 1-[2,3,8,8-tetramethyl-1,3,4,5,6,7- | B | 14.00% | 2 |
| | hexanhydronaphthalen-2-yl]ethanone | | | |
| MAJANTOL ® | beta,beta-3-Trimethyl- | B | 8.00% | 2 |
| | benzenepropanol | | | |
| PATCHOULI | Patchouli oil | Essence oil | 0.80% | 2 |
| OIL DECOL. | | | | |

The above listed fragrance composition was able to convey a sensual mood.

### 2.8 Fragrance Composition Conveying a Self-Confident Mood

TABLE 9

| Name | Chemical name | Group | [wt.-%] | Actimood score |
|---|---|---|---|---|
| ALDEHYDE C14 | 5-heptyltetrahydrofuran-2-one | D | 0.95% | 2 |
| SO-CALLED | | | | |
| APRIFLOREN ® | 5-Hexyldihydro-4-methylfuran- | D | 3.33% | 2 |
| | 2(3H)-one | | | |
| BENZYL | benzyl 2-hydroxybenzoate | A | 9.52% | 1 |
| SALICYLATE | | | | |
| BOURGEONAL | 3-(4-tert-butylphenyl)propanal | A | 0.95% | 2 |
| CARYOPHYLLENE | 4,11,11-trimethyl-8-methylene- | E | 1.90% | 3 |
| NAT. RECT. | bicyclo[7.2.0]undec-4-ene | | | |
| CASHMERAN | 1,1,2,3,3,-pentamethyl-2,5,6,7- | B | 1.90% | 2 |
| | tetrahydroinden-4-one | | | |
| CITRONELLOL | 3,7-dimethyloct-6-en-1-ol | A | 2.86% | 1 |
| 950 | | | | |
| ETHYLENE | 1,4-dioxacycloheptadecane- | C | 47.05% | 2 |
| BRASSYLATE | 5,17-dione | | | |
| HELVETOLIDE | [2-[1-(3,3- | C | 19.05% | 2 |
| | dimethylcyclohexyl)ethoxy]-2- | | | |
| | methyl-propyl]propanoate | | | |
| HYDROXY | 7-hydroxy-3,7-dimethyl-octanal | A | 4.76% | 1 |
| CITRONELLAL | | | | |
| IONONE ALPHA | (E)-4-(2,6,6-trimethylcyclohex- | A | 0.95% | 1 |
| | 2-en-1-yl)but-3-en-2-one | | | |
| LEAFOVERT ® | (Z)-Hex-3-en-1-yl methyl | E | 0.05% | 0 |
| | carbonate | | | |
| MAJANTOL ® | beta,beta-3-Trimethyl- | A | 3.81% | 2 |
| | benzenepropanol | | | |
| METHYL | Methyl anthranilate | Other | 0.05% | 0 |
| ANTHRANILATE | | | | |
| PHENYLETHYL | 2-phenylethanol | A | 2.86% | 1 |
| ALCOHOL BA | | | | |
| FREE | | | | |

The above listed fragrance composition was able to convey a self-confident mood.

### 2.9 Fragrance Composition Conveying a Well-being Mood

TABLE 10

| Name | Chemical name | Group | [wt.-%] | Actimood score |
|---|---|---|---|---|
| AMBROXIDE | 3a,6,6,9a- | B | 0.20% | 3 |
| | Tetramethyldodecahydronaphtho[2,1- | | | |
| | b]furan | | | |
| BERGAMOT | Bergamot oil | Essence | 15.00% | 3 |
| ECO ESSENCE | | oil | | |
| (E0636) W/O | | | | |
| MYRCENE | | | | |
| CALONE | 7-methyl-1,5-benzodioxepin-3-one | A | 0.10% | 3 |
| CASHMERAN | 1,1,2,3,3,-pentamethyl-2,5,6,7- | B | 0.50% | 1 |
| | tetrahydroinden-4-one | | | |
| CEDRAMBER | Cedrol methyl ether | Other | 2.50% | 0 |
| DIPROPYLENE | dipropylene glycol | Other | 36.80% | |
| GLYCOL | | | | |
| ELEMI OIL | Elemi oil | Essence | 0.20% | 1 |
| | | oil | | |
| GLOBALIDE ® | Oxacyclohexadecen-2-one | C | 15.00% | 2 |
| HEDIONE | methyl-2-[RAC-(1S,2S)-3-oxo-2- | A | 15.00% | 0 |
| | pentyl-cyclopentyl]acetate | | | |
| HELIOTROPIN | 1,3-benzodioxole-5-carbaldehyde | A | 0.30% | 2 |
| HEXENOL | Hexenol | Other | 0.50% | 0 |
| CIS-3 10% | | | | |
| DPG | | | | |
| HEXENYL | Hexenyl acetat | Other | 0.35% | 0 |
| ACETATE CIS- | | | | |
| 3 10% DPG | | | | |
| IONONE BETA | (E)-4-(2,6,6-trimethylcyclohexen-1- | A | 1.50% | 3 |
| | yl)but-3-en-2-one | | | |
| ISO E SUPER | 1-[2,3,8,8-tetramethyl-1,3,4,5,6,7- | B | 5.00% | 2 |
| | hexanhydronaphthalen-2- | | | |
| | yl]ethanone | | | |
| LEAFOVERT ® | (Z)-Hex-3-en-1-yl methyl carbonate | E | 0.15% | 2 |
| 10% DPG | | | | |
| LEMON OIL | lemon oil | essential | 2.50% | 0 |
| WINTER | | oil | | |
| BERGAPTEN | | | | |
| FREE | | | | |
| LEMONGRASS | lemongrass oil | essential | 0.10% | 1 |
| OIL RED | | oil | | |
| MADA | | | | |
| LILYBELLE ® | (+)-3-(4-Isopropylcyclohexen-1- | A | 0.10% | 0 |
| | yl)propanal | | | |
| LINALOOL | (6E)-3,7-dimethylnona-1,6-dien-3- | A | 1.50% | 0 |
| | ol | | | |
| MACROLIDE ® | Pentadecan-15-olide | C | 1.50% | 2 |
| SUPRA | | | | |
| MANDARIN | Mandarin oil | Essential | 0.10% | 2 |
| OIL | | oil | | |
| EXPRESSED | | | | |
| MADA | | | | |
| MANDARIN | Mandarin oil | Essential | 0.40% | 2 |
| OIL ITAL. | | oil | | |
| PEPPER | Pepper black oil | Essential | 0.40% | 2 |
| BLACK FRESH | | oil | | |
| OIL MADA | | | | |
| VANILLA ABS. | vanilla absoule | Essential | 0.10% | 3 |
| BOURBON | | oil | | |
| MADA 10% | | | | |
| DPG | | | | |
| VERTOCITRAL | 2,4-Dimethylcyclohex-3-ene-1- | Other | 0.10% | 0 |
| | carbaldehyde | | | |
| VETIVERYL | Complex mix | Essential | 0.10% | 1 |
| ACETATE | | oil | | |
| EXTRA | | | | |

The above listed fragrance composition was able to convey a well-being mood.

### 2.10 Fragrance Composition Conveying an Attractive Mood

TABLE 11

| Name | Chemical name | Group | [wt.-%] | Actimood score |
|---|---|---|---|---|
| AMBROXIDE | 3a,6,6,9a- | B | 7.14% | 3 |
| | Tetramethyldodecahydronaphtho[2,1- | | | |
| | b]furan | | | |
| BOURGEONAL | 3-(4-tert-butylphenyl)propanal | A | 1.43% | 3 |
| CASHMERAN | 1,1,2,3,3,-pentamethyl-2,5,6,7- | B | 14.29% | 3 |
| | tetrahydroinden-4-one | | | |
| COUMARIN | chromen-2-one | C | 1.43% | 3 |
| DIHYDRO | 2,6-dimethyloct-7-en-2-ol | A | 7.14% | 3 |
| MYRCENOL | | | | 3 |
| ETHYL | (6E)-3,7-dimethylnona-1,6-dien-3-ol | A | 24.29% | 2 |
| LINALOOL | | | | |
| ETHYL | 2-ethyl-3-hydroxy-pyran-4-one | C | 5.71% | 3 |
| MALTOL | | | | |
| ETHYL | 3-ethoxy-4-hydroxy-benzaldehyde | C | 7.14% | 3 |
| VANILLIN | | | | |
| ETHYLENE | 1,4-dioxacycloheptadecane-5,17- | C | 17.14% | 3 |
| BRASSYLATE | dione | | | |
| GLOBALIDE ® | Oxacyclohexadecen-2-one | C | 2.86% | 3 |
| HYDROXY | 7-hydroxy-3,7-dimethyl-octanal | A | 7.14% | 3 |
| CITRONELLAL | | | | |
| LAVENDER | Lavender oil | Essence | 1.43% | 2 |
| OIL MONT | | oil | | |
| BLANC | | | | |
| 40/42% NEW | | | | |
| MACROLIDE ® | Pentadecan-15-olide | C | 2.86% | 3 |
| SUPRA | | | | |

The above listed fragrance composition was able to convey an attractive mood.

### 2.11 Fragrance Composition Conveying a Fresh Mood

TABLE 12

| Name | Chemical name | Group | [wt.-%] | Actimood score |
|---|---|---|---|---|
| AMAROCIT ® | 6,6-Dimethoxy-2,5,5-trimethylhex-2- | D | 5.56% | 3 |
| | ene | | | |
| AMBROXIDE | 3a,6,6,9a- | B | 1.11% | 3 |
| | Tetramethyldodecahydronaphtho[2,1- | | | |
| | b]furan | | | |
| BERGAMOT | Bergamot oil | Essence | 22.22% | 3 |
| ECO ESSENCE | | oil | | |
| (E0636) W/O | | | | |
| MYRCENE | | | | |
| CALONE | 7-methyl-1,5-benzodioxepin-3-one | A | 1.11% | 3 |
| CITRAL FF | (2E)-3,7-Dimethylocta-2,6-dienal | D | 1.67% | 3 |
| CYCLAMEN | 3-(4-isopropylphenyl)-2-methyl- | A | 6.67% | 3 |
| ALDEHYDE | propanal | | | |
| ETHYL | (6E)-3,7-dimethylnona-1,6-dien-3-ol | A | 22.22% | 3 |
| LINALOOL | | | | |
| FLORALOZONE | 3-(4-ethylphenyl)-2,2- | A | 3.33% | 3 |
| | dimethylpropanal | | | |
| HEDIONE | methyl-2-[RAC-(1S,2S)-3-oxo-2- | A | 33.33% | 2 |
| | pentyl-cyclopentyl]acetate | | | |
| LAVENDER | lavender oil | Essence | 1.67% | 3 |
| OIL MONT | | oil | | |
| BLANC | | | | |
| 40/42% NEW | | | | |
| LILYBELLE ® | (+)-3-(4-Isopropylcyclohexen-1- | A | 1.11% | 3 |
| | yl)propana | | | |

The above listed fragrance composition was able to convey a fresh mood.

1-15. (canceled)

16. A method for imparting a positive mood in a subject comprising:
(a) providing a fragrance composition comprising:
(i) 0.01 to 35 wt. % of at least one compound of Group A having a bloomy odor impression selected from 3-(4-tert-butylphenyl)propanal, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-Trimethyl-benzenepropanol, methyl-2-aminobenzoate, (E)-4-(2,6,6-trimethylcyclohex-2-en-1-yl)but-3-en-2-one, 3-(4-ethylphenyl)-2,2-dimethylpropanal, 3-(1,3-benzodioxol-5-yl)-2-methyl-propanal, (2E)-3,7-dimethylocta-2,6-dien-1-ol, methyl-2-[RAC-(1S,2S)-3-oxo-2-pentyl-cyclopentyl]acetate, 1-phenylethyl acetate, benzyl acetate, 1,3-benzodioxole-5-carbaldehyde, [(Z)-hex-3-enyl]-2-hydroxybenzoate, (methyl-2-[RAC-(1 S,2S)-3-oxo-2-pentyl-cyclopentyl]acetate, (1,5-dimethyl-1-vinyl-hex-4-enyl)acetate, 2-phenylethanol, 4-Isopentylcyclohexanol, benzyl 2-hydroxybenzoate, 4-cyclohexylbutan-2-ol, (E)-1-(2,6,6-trimethylcyclohexan-1,3-dien-1-yl)but-2-en-1-one, (2E)-3,7-dimethylocta-2,6-dienyl]acetate, (E)-4-(2,6,6-trimethylcyclohexen-1-yl)but-3-en-2-one, (+)-3-(4-Isopropylcyclohexen-1-yl)propanal, 2,6-dimethyloct-7-en-2-ol 7-methyl-1,5-benzodioxepin-3-one, 1-methyl-4-(4-methylpent-3-enyl)cyclohex-3-ene-1-carbaldehyde, (E)-1-(2,6,6-trimethylcyclohex-2-en-1-yl)but-2-en-1-one, 2-isobutyl-4-methyl-tetrahydropyran-4-ol, [(2E)-3,7-dimethylocta-2,6-dienyl]acetate, indole, (1 E)-1-(2,6,6-trimethylcyclohex-2-en-1-yl)hepta-1,6-dien-3-one, methyl 2-[(7-hydroxy-3,7-dimehyloctylidene)amino]benzoate, rac-(e)-1-[rac-(1 r,2s)-2,6,6-trimethylcyclohex-3-en-1-yl]but-2-en-1-one, 2-methyl-1-phenyl-propan-2-ol, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 2,3-dihydro-1,1-dimethyl-1 h-indene-ar-propanal, (E)-1-(2,4,4-trimethylcyclohexen-1-yl)but-2-en-1-one, (2E)-2-methyl-6-methylene-octa-2,7-dien-1-ol, 5-hexyl-5-methyl-tetrahydrofuran-2-one, 3,7-dimethylocta-1,6-dien-3-ol, 4-(propan-2-yl)cyclohexyl]methanol, 1-naphthalen-2-ylethanone, 2-ethoxynaphtalene, 2-methoxynaphtalene, 3-methyl-2-benzofuran-1-yl)ethanone, 2-phenoxyethyl 2-methylpropanoate, 3-methyl-5-phenylpentan-1-ol, 2-methyl-4-phenyl-butan-2-ol, (2,2,2-trichloro-1-phenyl-ethyl) acetate, 2-methyl-5-phenylpentanol, rac-(2r,4s)-4-methyl-2-(2-methylprop-1-enyl)tetrahydropyran, 2,2,5-trimethyl-5-pentyl-cyclopentanone, 3-(4-methoxyphenyl)-2-methyl-propanal, (6E)-3,7,11-trimethyldodeca-1,6,10-trien-3-ol, (6Z)-3,7,11-trimethyldodeca-1,6,10-trien-3-ol, and mixtures thereof, and
(ii) 0.01 to 35 wt. % of at least one compound of Group B having a woody and/or balsam odor impression selected from 3a,6,6,9a-tetramethyldodecahydronaphtho[2,1-b]furan, 1,1,2,3,3,-pentamethyl-2,5,6,7-tetrahydroinden-4-one, 8-methoxy-2,6,6,8-tetramethyl-tricyclo[5.3.1.01,5]undecane, (E)-3-methyl-4-(2,6,6-trimethylcyclohex-2-en-1-yl)but-3-en-2-one, (2E)-2-Ethyl-4-(2,2,3)-trimethylcyclopent-3-en-1-yl)but-2-en-1-ol, 1-[(2E,5E,9E)-2,6,10-trimethylcyclododeca-2,5,9-trien-1-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, 1-[2,3,8,8-tetramethyl-1,3,4,5,6,7-hexanhydronaphthalen-2-yl]ethanone, (Ethoxymethoxy)cyclododecane, dioxol-4H-4A,9-methanoazuleno(5,6-D)-octahydro-2-propyl-2,5,8,8,9A-pentamethyl-1,3, (E/Z)-4-Ethyloct-2/3-enenitrile, 3,4-dimethylbenzonitrile, (2E)-3-phenylprop-2-en-1-ol, 2,3,6-trimethylcyclohexanol, oxacycloheptadec-10-en-2-one, (4aR,5R,7aS,9R)-octahydro-2,2,5,8,8,9a-hexamethyl-4H-4a,9-methanoazuleno(5,6-d)-1,3-dioxole, 2-ethoxy-2,6,6-trimethyl-9-methylene-bicyclo[3.3.1]nonane, (E)-2-methyl-3-phenyl-prop-2-en-1-ol, rac-(E,2R,3R)-3-methyl-5-(2,2,3-trimethylcyclopent-3-en-1-yl)pent-4-en-2-ol, 4-(1,7,7-trimethyl-2-bucyclo[3.1.1]heptanyl)ccyclohexan-1-ol, 6-butan-2-ylquinoline, 4-(1-ethoxyvinyl)-3,3,5,5-tetramethyl-cyclohexanone, (E)-3,4,5,6,6-pentamethylhept-3-en-2-one, 2-(4-methylcyclohex-3-en-1-yl)propan-2-ol, 1-(2,6,6,8-tetramethyl-9-tricyclo[5.3.1.01,5]undec-8-enyl)ethanone, rac-(3′R,6′R,9′R)-2′,2′,8′,8′-tetramethylspiro[1,3-dioxolane-2,10′-octahydro-1 h-2,4a-methanonapthalene], 1-(1,1,2,4,4,7-hexamethyltetralin-6-yl)ethenone, and mixtures thereof, and
(iii) 0.01 to 35 wt. % of at least one compound of Group C having an earthy and/or sweet odor impression selected from chromen-2-one, 2-ethyl-3-hydroxy-pyran-4-one, (5Z)-3-methylcyclopentadec-5-en-1-one, 3-ethoxy-4-hydroxy-benzaldehyde, 1,4-dioxacycloheptadecane-5,17-dione, 4,6,6,7,8,8-hexamethyl-1,3,4,7-tetrahydrocyclopenta[G]isochromene, oxacyclohexadecen-2-one (mixture of isomers), Pentadecan-15-olide, [2-[1-(3,3-dimethylcyclohexyl)ethoxy]-2-methyl-propyl]propanoate, 3,6-Dimethyl-3H-benzofuran-2-one, (5Z)-3-methylcyclopentadec-5-en-1-one, 4,6,6,7,8,8-hexamethyl-1,3,4,7-tetrahydrocyclopenta[G]isochromene, 2-methyl-5-propyl-cyclohex-2-en-1-one, 1,3-benzodioxole-5-carbaldehyde, 1-pyrazin-2-ylethanone, 5-pentyltetrahydrofuran-2-one, 1-methoxy-4-[(e)-prop-1-enyl]benzene, 4-methoxybenzaldehyde, 4-hydroxy-2,5-dimethylfuran-3-one, 3-hydroxy-2-methyl-4h-pyran-4-one, 2-(4-methylthiazol-5-yl)ethanol, 4-hydroxy-3-methoxy-benzaldehyde, (4-formyl-2-methoxy-phenyl)-2-methylpropanoate, and mixtures thereof; and
(iv) at least one essential oil and/or essence and/or absolute selected from bergamot oil, ginger root extract, vetiver oil, lemon oil, artemisia oil, cinnamon bark oil, elemi oil, ginger oil, lemongrass oil, longoza oil, mandarin oil, pepper black oil, vanilla absolue, red berry extract, bay oil, cedarwood oil, hemp oil, pink pepper leaf oil, amyris oil, cardamom oil, carrot seed oil, cypress oil, davana oil, geranium oil, grapefruit oil, lime oil, olibanum oil, palmarosa oil, sage oil, sandalwood oil, thyme oil, artichoke essence, asparagus essence, cauliflower essence, hemp essence, leek essence, clove essence, passionfruit essence, amber base, red apple essence, benzaldehyde, Styrax tonkinensis essence, camphor oil, eucalyptus oil, jasmin oil, jasmin absolue, orange oil, rosemary oil, galbanum essence, orange flower absolue, patchouli essence, rose absolue, tonka bean absolue, tuberose absolue, rose bourboniana absolute, and mixtures thereof; and
(ii) contacting the subject with the fragrance composition in an amount sufficient to enhance the mood of the subject.

17. The method of claim 16, wherein the fragrance composition further comprises:
(v) 0.2 to 20 wt. % of at least one compound of Group D having a fruity odor impression selected from allyl 3-cyclohexylpropanoate, 5-hexyltetrahydrofuran-2-one, methoxyphenylbutanone, 2,6-dimethyloct-7-en-2-ol, (2E)-3,7-Dimethylocta-2,6-dienal, 7-methyl-1,5-benzodioxepin-3-one, 5-heptyltetrahydrofuran-2-one, hexyl acetate, isopropyl tetradeconate, 6,6-dimethoxy-2,5,5-trimethylhex-2-ene, ethyl-3-oxobutanoate, ethyl-2-methylpentanoate, 5-hexyldihydro-4-methylfuran-2(3H)-one, 2-tert-butylcyclohexyl acetate, diethyl 2-methylenebutanedioate, 2-bicyclo[2.2.1]hept-5-enyl acetate, ethyl 3-oxobutanoate, linolal, mixture of diethyl (E/Z)-2-methylbut-2-enedioate and diethyl 2-methylenebutanedioate, mixture of (5-isopropenyl-2-methyl-cyclohex-2-en-1-yl) acetate and (5-isopropenyl-2-methyl-cyclohexyl) acetate, ethyl tetrahydrofuran-2-carboxylate, decanal, ethyl rac-(2r,3r)-3-methyl-3-phenyl-oxirane-2-carboxylate, allyl hexanoate, (1,1-dimethyl-2-phenyl-ethyl) acetate, 5-octyltetrahydrofuran-2-one, ethyl butanoate, ethyl 2-methylbutanoate, 3-methylbutyl acetate, ethyl 2-(2-methyl-1,3-dioxolan-2-yl)acetate, 2,4-dimethyl-4,4a,5,9b-tetrahydoindeno[1,2-d][1,3]dioxine, 2-methyl-4-propyl-1,3-oxathiane, 2,4,6-trimethyl-4-phenyl-1,3-dioxane, allyl heptanoate, and mixtures thereof.

18. The method of claim 16, wherein the fragrance composition further comprises:
(v) 0.01 to 35 wt. % of at least one compound of Group E having green and/or herbal odor impression selected from allyl-2isopentyloxyacetate, allyl (cyclohexyloxy)acetate, 1-(5,5-dimethylcyclohexen-1-yl)pent-4-en-1-one, 2,4-dimethylcyclohex-3-ene-1-carbaldehyde, (Z)-hex-3-en-1-yl methyl carbonate, hex-5-enyl 2-methylbutanoate, (Z)-hex-3-en-1-ol, [(Z)-hex-3-enyl]acetate, 4,11,11-trimethyl-8-methylene-bicyclo[7.2.0]undec-4-ene, methyl-2,4-dihydroxy-3,6-dimethyl-benzoate, allyl (cyclohexyloxy)acetate, 3,3,5-trimethylcyclohexyl acetate, 1-methyl-4-(4-methylpent-3-enyl)cyclohex-3-ene-1-carbaldehyde, undec-10-enal, 5-isopropenyl-2-methyl-cyclohex-2-en-1-one, 2,4,4,7-tetramethyloct-6-en-3-one, 4-allyl-2-methoxy-phenol, 3a,4,5,6,7,7a-hexahydro-4,7-methanoinden-6-yl acetate, 2,6-dimethylhept-5-enal, 2-ethoxy-4-(methoxymethyl)phenol, methyl 2-nonynoate, (4-tert-butylcyclohexyl) acetate, 5-methylheptan-3-one oxime, (E)-4-methyldec-3-en-5-ol, and mixtures thereof.

19. The method of claim 16, wherein the fragrance composition is contained in a perfumed product.

20. The method of claim 16, wherein the fragrance composition is contained in a perfumed product in an amount of 0.01 to 35 wt. %, based on a total weight of the perfumed product.

21. The method of claim 16, wherein the fragrance composition is contained in a perfumed product and the perfumed product is selected from a fine fragrance, a personal cosmetic, a toiletry, and a healthcare product.

22. The method of claim 16, wherein the fragrance composition comprises:
(i) 0.2 to 20 wt. % of the at least one compound of Group A;
(ii) 0.2 to 20 wt. % of the at least one compound of Group B; and
(iii) 0.2 to 20 wt. % of the at least one compound of Group C.

23. The method of claim 16, wherein the fragrance composition comprises:
(i) 0.5 to 10 wt. % of the at least one compound of Group A;
(ii) 0.5 to 10 wt. % of the at least one compound of Group B; and
(iii) 0.5 to 10 wt. % of the at least one compound of Group C.

24. A fragrance composition for conveying a positive mood in a subject comprising:
(i) 0.01 to 35 wt. % of at least one compound of Group A having a bloomy odor impression selected from 3-(4-tert-butylphenyl)propanal, 7-hydroxy-3,7-dimethyl-octanal, 3,7-dimethyloct-6-en-1-ol, 3-(4-isopropylphenyl)-2-methyl-propanal, (6E)-3,7-dimethylnona-1,6-dien-3-ol, beta,beta-3-Trimethyl-benzenepropanol, methyl-2-aminobenzoate, (E)-4-(2,6,6-trimethylcyclohex-2-en-1-yl)but-3-en-2-one, 3-(4-ethylphenyl)-2,2-dimethylpropanal, 3-(1,3-benzodioxol-5-yl)-2-methyl-propanal, (2E)-3,7-dimethylocta-2,6-dien-1-ol, methyl-2-[RAC-(1S,2S)-3-oxo-2-pentyl-cyclopentyl]acetate, 1-phenylethyl acetate, benzyl acetate, 1,3-benzodioxole-5-carbaldehyde, [(Z)-hex-3-enyl]-2-hydroxybenzoate, (methyl-2-[RAC-(1S,2S)-3-oxo-2-pentyl-cyclopentyl]acetate, (1,5-dimethyl-1-vinyl-hex-4-enyl)acetate, 2-phenylethanol, 4-Isopentylcyclohexanol, benzyl 2-hydroxybenzoate, 4-cyclohexylbutan-2-ol, (E)-1-(2,6,6-trimethylcyclohexan-1,3-dien-1-yl)but-2-en-1-one, (2E)-3,7-dimethylocta-2,6-dienyl]acetate, (E)-4-(2,6,6-trimethylcyclohexen-1-yl)but-3-en-2-one, (+)-3-(4-Isopropylcyclohexen-1-yl)propanal, 2,6-dimethyloct-7-en-2-ol 7-methyl-1,5-benzodioxepin-3-one, 1-methyl-4-(4-methylpent-3-enyl)cyclohex-3-ene-1-carbaldehyde, (E)-1-(2,6,6-trimethylcyclohex-2-en-1-yl)but-2-en-1-one, 2-isobutyl-4-methyl-tetrahydropyran-4-ol, [(2E)-3,7-dimethylocta-2,6-dienyl]acetate, indole, (1 E)-1-(2,6,6-trimethylcyclohex-2-en-1-yl)hepta-1,6-dien-3-one, methyl 2-[(7-hydroxy-3,7-dimehyloctylidene)amino]benzoate, rac-(e)-1-[rac-(1 r,2s)-2,6,6-trimethylcyclohex-3-en-1-yl]but-2-en-1-one, 2-methyl-1-phenyl-propan-2-ol, ethyl 2,6,6-trimethylcyclohexa-1,3-diene-1-carboxylate, 3-(3-isopropylphenyl)butanal, 2,6-dimethylheptan-2-ol, 2-(phenylmethylidene)octanal, 2,3-dihydro-1,1-dimethyl-1 h-indene-ar-propanal, (E)-1-(2,4,4-trimethylcyclohexen-1-yl)but-2-en-1-one, (2E)-2-methyl-6-methylene-octa-2,7-dien-1-ol, 5-hexyl-5-methyl-tetrahydrofuran-2-one, 3,7-dimethylocta-1,6-dien-3-ol, 4-(propan-2-yl)cyclohexyl]methanol, 1-naphthalen-2-ylethanone, 2-ethoxynaphtalene, 2-methoxynaphtalene, 3-methyl-2-benzofuran-1-yl)ethanone, 2-phenoxyethyl 2-methylpropanoate, 3-methyl-5-phenylpentan-1-ol, 2-methyl-4-phenyl-butan-2-ol, (2,2,2-trichloro-1-phenyl-ethyl) acetate, 2-methyl-5-phenylpentanol, rac-(2r,4s)-4-methyl-2-(2-methylprop-1-enyl)tetrahydropyran, 2,2,5-trimethyl-5-pentyl-cyclopentanone, 3-(4-methoxyphenyl)-2-methyl-propanal, (6E)-3,7,11-trimethyldodeca-1,6,10-trien-3-ol, (6Z)-3,7,11-trimethyldodeca-1,6,10-trien-3-ol, and mixtures thereof, and
(ii) 0.01 to 35 wt. % of at least one compound of Group B having a woody and/or balsam odor impression selected from 3a,6,6,9a-tetramethyldodecahydronaphtho[2,1-b]furan, 1,1,2,3,3,-pentamethyl-2,5,6,7-tetrahydroinden-4-one, 8-methoxy-2,6,6,8-tetramethyl-tricyclo[5.3.1.01,5]undecane, (E)-3-methyl-4-(2,6,6-trimethylcyclohex-2-en-1-yl)but-3-en-2-one, (2E)-2-Ethyl-4-(2,2,3)-trimethylcyclopent-3-en-1-yl)but-2-en-1-ol, 1-[(2E,5E,9E)-2,6,10-trimethylcyclododeca-2,5,9-trien-1-yl]ethanone, 2,2,6,8-tetramethyltricyclo[5.3.1.03,8]undecan-3-ol, 3-methyl-5-(2,2,3-trimethyl-1-cyclopent-3-enyl)pentan-2-ol, 1-[2,3,8,8-tetramethyl-1,3,4,5,6,7-hexanhydronaphthalen-2-yl]ethanone, (Ethoxymethoxy)cyclododecane, dioxol-4H-4A,9-methanoazuleno(5,6-D)-octahydro-2-propyl-2,5,8,8,9A-pentamethyl-1,3, (E/Z)-4-Ethyloct-2/3-enenitrile, 3,4-dimethylbenzonitrile, (2E)-3-phenylprop-2-en-1-ol, 2,3,6-trimethylcyclohexanol, oxacycloheptadec-10-en-2-one, (4aR,5R,7aS,9R)-octahydro-2,2,5,8,8,9a-hexamethyl-4H-4a,9-methanoazuleno(5,6-d)-1,3-dioxole, 2-ethoxy-2,6,6-trimethyl-9-methylene-bicyclo[3.3.1]nonane, (E)-2-methyl-3-phenyl-prop-2-en-1-ol, rac-(E,2R,3R)-3-methyl-5-(2,2,3-trimethylcyclopent-3-en-1-yl)pent-4-en-2-ol, 4-(1,7,7-trimethyl-2-bucyclo[3.1.1]heptanyl)ccyclohexan-1-ol, 6-butan-2-ylquinoline, 4-(1-ethoxyvinyl)-3,3,5,5-tetramethyl-cyclohexanone, (E)-3,4,5,6,6-pentamethylhept-3-en-2-one, 2-(4-methylcyclohex-3-en-1-yl)propan-2-ol, 1-(2,6,6,8-tetramethyl-9-tricyclo[5.3.1.01,5]undec-8-enyl)ethanone, rac-(3′R,6′R,9′R)-2′,2′,8′,8′-tetramethylspiro[1,3-dioxolane-2,10′-octahydro-1 h-2,4a-methanonapthalene], 1-(1,1,2,4,4,7-hexamethyltetralin-6-yl)ethanone and mixtures thereof; and
(iii) 0.01 to 35 wt. % of at least one compound of Group C having an earthy and/or sweet odor impression selected from chromen-2-one, 2-ethyl-3-hydroxy-pyran-4-one, (5Z)-3-methylcyclopentadec-5-en-1-one, 3-ethoxy-4-hydroxy-benzaldehyde, 1,4-dioxacycloheptadecane-5,17-dione, 4,6,6,7,8,8-hexamethyl-1,3,4,7-tetrahydrocyclopenta[G]isochromene, oxacyclohexadecen-2-one (mixture of isomers), Pentadecan-15-olide, [2-[1-(3,3-dimethylcyclohexyl)ethoxy]-2-methyl-propyl]propanoate, 3,6-Dimethyl-3H-benzofuran-2-one, (5Z)-3-methylcyclopentadec-5-en-1-one, 4,6,6,7,8,8-hexamethyl-1,3,4,7-tetrahydrocyclopenta[G]isochromene, 2-methyl-5-propyl-cyclohex-2-en-1-one, 1,3-benzodioxole-5-carbaldehyde, 1-pyrazin-2-ylethanone, 5-pentyltetrahydrofuran-2-one, 1-methoxy-4-[(e)-prop-1-enyl]benzene, 4-methoxybenzaldehyde, 4-hydroxy-2,5-dimethylfuran-3-one, 3-hydroxy-2-methyl-4h-pyran-4-one, 2-(4-methylthiazol-5-yl)ethanol, 4-hydroxy-3-methoxy-benzaldehyde, (4-formyl-2-methoxy-phenyl)-2-methylpropanoate, and mixtures thereof; and
(iv) at least one essential oil and/or essence and/or absolute selected from bergamot oil, ginger root extract, vetiver oil, lemon oil, artemisia oil, cinnamon bark oil, elemi oil, ginger oil, lemongrass oil, longoza oil, mandarin oil, pepper black oil, vanilla absolue, red berry extract, bay oil, cedarwood oil, hemp oil, pink pepper leaf oil, amyris oil, cardamom oil, carrot seed oil, cypress oil, davana oil, geranium oil, grapefruit oil, lime oil, olibanum oil, palmarosa oil, sage oil, sandalwood oil, thyme oil, artichoke essence, asparagus essence, cauliflower essence, hemp essence, leek essence, clove essence, passionfruit essence, amber base, red apple essence, benzaldehyde, Styrax tonkinensis essence, camphor oil, eucalyptus oil, jasmin oil, jasmin absolue, orange oil, rosemary oil, galbanum essence, orange flower absolue, patchouli essence, rose absolue, tonka bean absolue, tuberose absolue, rose bourboniana absolute.

25. The fragrance composition of claim 24, further comprising:
(v) 0.2 to 20 wt. % of at least one compound of Group D having a fruity odor impression selected from allyl 3-cyclohexylpropanoate, 5-hexyltetrahydrofuran-2-one, methoxyphenylbutanone, 2,6-dimethyloct-7-en-2-ol, (2E)-3,7-Dimethylocta-2,6-dienal, 7-methyl-1,5-benzodioxepin-3-one, 5-heptyltetrahydrofuran-2-one, hexyl acetate, isopropyl tetradeconate, 6,6-dimethoxy-2,5,5-trimethylhex-2-ene, ethyl-3-oxobutanoate, ethyl-2-methylpentanoate, 5-hexyldihydro-4-methylfuran-2(3H)-one, 2-tert-butylcyclohexyl acetate, diethyl 2-methylenebutanedioate, 2-bicyclo[2.2.1]hept-5-enyl acetate, ethyl 3-oxobutanoate, linolal, mixture of diethyl (E/Z)-2-methylbut-2-enedioate and diethyl 2-methylenebutanedioate, mixture of (5-isopropenyl-2-methyl-cyclohex-2-en-1-yl) acetate and (5-isopropenyl-2-methyl-cyclohexyl) acetate, ethyl tetrahydrofuran-2-carboxylate, decanal, ethyl rac-(2r,3r)-3-methyl-3-phenyl-oxirane-2-carboxylate, allyl hexanoate, (1,1-dimethyl-2-phenyl-ethyl) acetate, 5-octyltetrahydrofuran-2-one, ethyl butanoate, ethyl 2-methylbutanoate, 3-methylbutyl acetate, ethyl 2-(2-methyl-1,3-dioxolan-2-yl)acetate, 2,4-dimethyl-4,4a,5,9b-tetrahydoindeno[1,2-d][1,3]dioxine, 2-methyl-4-propyl-1,3-oxathiane, 2,4,6-trimethyl-4-phenyl-1,3-dioxane, allyl heptanoate, and mixtures thereof.

26. The fragrance composition of claim 24, further comprising:
(v) 0.01 to 35 wt. % of at least one compound of Group E having green and/or herbal odor impression selected from allyl-2isopentyloxyacetate, allyl (cyclohexyloxy)acetate, 1-(5,5-dimethylcyclohexen-1-yl)pent-4-en-1-one, 2,4-dimethylcyclohex-3-ene-1-carbaldehyde, (Z)-hex-3-en-1-yl methyl carbonate, hex-5-enyl 2-methylbutanoate, (Z)-hex-3-en-1-ol, [(Z)-hex-3-enyl]acetate, 4,11,11-trimethyl-8-methylene-bicyclo[7.2.0]undec-4-ene, methyl-2,4-dihydroxy-3,6-dimethyl-benzoate, allyl (cyclohexyloxy)acetate, 3,3,5-trimethylcyclohexyl acetate, 1-methyl-4-(4-methylpent-3-enyl)cyclohex-3-ene-1-carbaldehyde, undec-10-enal, 5-isopropenyl-2-methyl-cyclohex-2-en-1-one, 2,4,4,7-tetramethyloct-6-en-3-one, 4-allyl-2-methoxy-phenol, 3a,4,5,6,7,7a-hexahydro-4,7-methanoinden-6-yl acetate, 2,6-dimethylhept-5-enal, 2-ethoxy-4-(methoxymethyl)phenol, methyl 2-nonynoate, (4-tert-butylcyclohexyl) acetate, 5-methylheptan-3-one oxime, (E)-4-methyldec-3-en-5-ol, and mixtures thereof.

27. The fragrance composition of claim 24, wherein the fragrance composition is contained in a perfumed product.

28. The fragrance composition of claim 24, wherein the fragrance composition is contained in a perfumed product in an amount of 0.01 to 35 wt. %, based on a total weight of the perfumed product.

29. The fragrance composition of claim 24, wherein the fragrance composition is contained in a perfumed product and the perfumed product is selected from a fine fragrance, a personal cosmetic, a toiletry, and a healthcare product.

30. The fragrance composition of claim 24, further comprising:
(i) 0.2 to 20 wt. % of the at least one compound of Group A;
(ii) 0.2 to 20 wt. % of the at least one compound of Group B; and
(iii) 0.2 to 20 wt. % of the at least one compound of Group C.

31. The fragrance composition of claim 24, further comprising:
(i) 0.5 to 10 wt. % of the at least one compound of Group A;
(ii) 0.5 to 10 wt. % of the at least one compound of Group B; and
(iii) 0.5 to 10 wt. % of the at least one compound of Group C.
